SEQUENCE LISTING (7 sequences)

SEQ ID NO: 1 - protein, 1273 aa, SARS-CoV-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - DNA, 2760 bp, SARS-CoV-2
  source 1..2760: mol_type = other DNA
gcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagttttttccttccatttcaggtgtcgtgtctagagcggccgatatcgaagccaccatgttcgtgttcctggtcctgctgccactcgtcagcagccagtgcgtcaatctgacaactcggactcagctgccacctgcttatactaatagcttcaccagaggcgtgtactatcctgacaaggtgtttagaagctccgtgctgcactctacacaggatctgtttctgccattctttagcaacgtgacctggttccacgccatcagcggcaccaatggcacaaagcggttcgacaatcccgtgctgccttttaacgatggcgtgtacttcgcctctaccgagaagagcaacatcatcagaggctggatctttggcaccacactggactccaagacacagtctctgctgatcgtgaacaatgccaccaacgtggtcatcaaggtgtgcgagttccagttttgtaatgatcccttcctgggcgtgtatcacaagaacaataagagctggatggagtccgagtttagagtgtattctagcgccaacaactgcacatttgagtacgtgagccagcctttcctgatggacctggagggcaagcagggcaatttcaagaacctgagggagttcgtgtttaagaatatcgacggctacttcaaaatctactctaagcacacccccatcaacctggtgcgcgacctgcctcagggcttcagcgccctggagcccctggtggatctgcctatcggcatcaacatcacccggtttcagacactgctggccctgcacagaagctacctgacacccggcgactcctctagcggatggaccgccggcgctgccgcctactatgtgggctacctccagccccggaccttcctgctgaagtacaacgagaatggcaccatcacagacgcagtggattgcgccctggaccccctgagcgagacaaagtgtacactgaagtcctttaccgtggagaagggcatctatcagacatccaatttcagggtgcagccaaccgagtctatcgtgcgctttcctaatatcacaaacctgtgcccatttggcgaggtgttcaacgcaacccgcttcgccagcgtgtacgcctggaataggaagcggatcagcaactgcgtggccgactatagcgtgctgtacaactccgcctctttcagcacctttaagtgctatggcgtgtcccccacaaagctgaatgacctgtgctttaccaacgtctacgccgattctttcgtgatcaggggcgacgaggtgcgccagatcgcccccggccagacaggcaagatcgcagactacaattataagctgccagacgatttcaccggctgcgtgatcgcctggaacagcaacaatctggattccaaagtgggcggcaactacaattatctgtaccggctgtttagaaagagcaatctgaagcccttcgagagggacatctctacagaaatctaccaggccggcagcaccccttgcaatggcgtggagggctttaactgttatttcccactccagtcctacggcttccagcccacatacggcgtgggctatcagccttaccgcgtggtggtgctgagctttgagctgctgcacgccccagcaacagtgtgcggccccaagaagtccaccaatctggtgaagaacaagtgcgtgaacttcaacttcaacggcctgaccggcacaggcgtgctgaccgagtccaacaagaagttcctgccatttcagcagttcggcagggacatcgacgataccacagacgccgtgcgcgacccacagaccctggagatcctggacatcacaccctgctctttcggcggcgtgagcgtgatcacacccggcaccaatacaagcaaccaggtggccgtgctgtatcagggcgtgaattgtaccgaggtgcccgtggctatccacgccgatcagctgaccccaacatggcgggtgtacagcaccggctccaacgtcttccagacaagagccggatgcctgatcggagcagagcacgtgaacaattcctatgagtgcgacatcccaatcggcgccggcatctgtgcctcttaccagacccagacaaactctcacagacgggcacgatagtgattaattaagcgccgtttctctcccatatggtcgaccgatcctgagaacttcagggtgagtttggggactcgatcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcagg

SEQ ID NO: 3 - protein, 669 aa, synthetic construct
  source 1..669: note = Amino acid sequence of ISR52
CCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRAR

SEQ ID NO: 4 - DNA, 26 bp, synthetic construct
  source 1..26: mol_type = other DNA; note = Synthetic primer
acaggtacgttaatagttaatagcgt

SEQ ID NO: 5 - DNA, 22 bp, synthetic construct
  source 1..22: mol_type = other DNA; note = Synthetic primer
atattgcagcagtacgcacaca

SEQ ID NO: 6 - DNA, 26 bp, synthetic construct
  source 1..26: mol_type = other DNA; note = Synthetic probe
acactagccatccttactgcgcttcg

SEQ ID NO: 7 - protein, 669 aa, synthetic construct
  source 1..669: note = Sequence of ISR52-DS
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRAR

(19) United States
(12) Patent Application Publication
(10) Pub. No.: US 2025/0186577 A1
(43) Pub. Date: Jun. 12, 2025

(54) VACCINE COMPOSITION COMPRISING AN ANTIGEN AND A TLR3 AGONIST

(71) Applicant: ISR IMMUNE SYSTEM REGULATION HOLDING AB (PUBL), Solna (SE)

(72) Inventors: Ola WINQVIST, Uppsala (SE); Johan SJÖDAHL, Stockholm (SE)

(21) Appl. No.: 18/843,185

(22) PCT Filed: Mar. 1, 2023
(86) PCT No.: PCT/EP2023/055188

§ 371 Date: Aug. 30, 2024

(30) Foreign Application Priority Data
Mar. 2, 2022 (EP) 22159691.9

(51) Int. Cl. A61K 39/215; A61K 9/00; A61K 31/07; A61K 31/593; A61K 39/00; A61K 39/39; A61K 47/18; A61K 47/26; A61K 47/34; A61K 47/40; A61P 31/14

(52) U.S. Cl. CPC A61K 39/215; A61K 9/0043; A61K 9/007; A61K 31/07; A61K 31/593; A61K 39/39; A61K 47/183; A61K 47/26; A61K 47/34; A61K 47/40; A61P 31/14; A61K 2039/543; A61K 2039/544; A61K 2039/55561

(57) ABSTRACT

A vaccine composition comprising one or more proteins expressed on the surface of a respiratory virus or bacterium and one or more pharmaceutically acceptable excipient, wherein the composition is in particulate form having a mean particle size in a range of from 2 to 50 μm. The protein is contained in the composition in its correctly folded three-dimensional structure.

### FIELD OF THE INVENTION

The present invention relates to the fields of medical science, immunology and vaccines. The present invention provides vaccine kits and compositions capable of stimulating the immune system, e.g., against pathogenic respiratory bacteria and pathogenic respiratory viruses. The present invention also provides methods for administration the vaccines so that the individual obtains immunity from pathogenic bacteria and viruses. In particular, the present invention relates to a vaccine composition against respiratory viruses or bacteria such as, e.g., SARS, MERS and COVID-19.

### BACKGROUND

Coronaviruses are a group of RNA viruses that cause diseases in mammals. They cause respiratory tract infections that can range from mild to lethal. Mild illnesses in humans include case of common cold, while more lethal varieties can cause SARS, MERS and COVID-19. Coronaviruses constitute the subfamily Orthocoronavirinae in the family Coronaviridae, order Nidovirales and realm Ribiviria. They are enveloped viruses with a positive-sense single-stranded RNA genome end a nucleocapsid of helical symmetry.

Severe acute respiratory syndrome coronavirus 2 (SARS-CoV2) is the strain of coronavirus that causes coronavirus disease 2019 (COVID 19), which is respiratory illness responsible for the COVID-19 pandemic. It is also referred to as 2019 novel coronavirus (2019-nCoV) or human coronavirus 2019 (HCoV-19 or hCoV-19). SARS-CoV2 is a Baltimore class IV positive-sense single-stranded RNA virus that is contagious in humas. It is the successor to SARS-CoV-1, the strain that caused the 2002-2004 SARS outbreak.

SARS-CoV2 is a strain of severe acute respiratory syndrome-related coronavirus (SARSr-CoV). It is believed to have zoonotic origins and has close genetic similarity to bat coronaviruses, suggesting it emerged from a bat-borne virus.

Many people suffering from corona or other viral or bacterial disease experiences disease-associated effects such as tiredness, less energy, muscle pain, joint pain, sore joints and bones etc. The effects do not necessary disappear when the main disease has been combatted. Thus about 10-20% of persons having been infected with COVID-19 experience late effects after several months.

WO 2021/178306 relates to an immunogenic composition for preventing SARS-COVID-2 infection. However, the immunogenic composition is different from a composition of the present invention and the immunogenic composition is administered intra-muscularly.

Recently, and reported in the Examples herein, the inventors have observed a significant better immune response in subjects suffering from a respiratory disease caused by COVID-19 when a vaccine composition was administered nasally or pulmonary. It is contemplated that the principle of administration a vaccine composition at the mucosal site where the virus or bacterium meets its host will be valid for all vaccines against respiratory diseases caused by a respiratory virus or bacterium.

Thus, there is a need for effective and safe new vaccines for preventing diseases originating from respiratory bacterial and viral infections. There is also a need for new adjuvants and optimized administration to achieve a better immune response following vaccination. Such new vaccines are required to have attractive combinations of properties including strong immune response when formulated into a product, and low toxicity. In particular, there is a need for such new vaccines in the field of viral infections where several MERS and SARS outbreaks have spread through several countries just during the last two decades. Such epidemics may have severe impacts also beyond those individuals attracting the virus, e.g. on travelling, hospitals, businesses and society at large.

During the pandemic COVID-19 situation it has become clear that there is a need for effective vaccines and therapies to combat diseases or to alleviate side-effects of diseases including viral diseases like coronaviruses such as COVID-19.

Hence, there is a need for effective and safe new vaccines for preventing diseases originating from bacterial and in particular viral infections in the respiratory system.

### SUMMARY OF THE INVENTION

In an aspect, the present invention relates to a vaccine composition comprising proteins expressed on the surface of respiratory viruses or bacteria for nasal administration or administration by inhalation. The composition is in solid form and administered in the form of a powder or a particulate composition. The protein in the composition functions as an antigen and in the composition, the protein is present in its correctly folded three-dimensional structure so that it is correctly recognized after administration. In embodiments, the respiratory virus is a coronavirus, and the antigen is a SARS-CoV-2 spike protein. The vaccine composition contains one or more pharmaceutically acceptable excipients. When the composition is in particulate form the particles have a mean particle size in a range of from 1 to 50 μm. The particle size depends on the administration route. Smaller particle sizes are suitable for administration to the lungs. The vaccine composition may also contain an adjuvant such as, e.g., a Toll-like receptor such as TLR3, and one or more immune-stimulating agents.

Although the Examples herein is directed to vaccine compositions for use in preventing COVID-19 infection, it is contemplated that the compositions can be formulated with other antigens as those against COVID-19, especially with antigens against respiratory diseases caused by bacteria or viruses. As described herein no side-effects relating to brain inflammation have been observed, and the nasal administration route seems to be advantageous to avoid such side-effects.

More specifically, the invention relates to a vaccine composition comprising one or more proteins expressed on the surface of a respiratory coronavirus and one or more pharmaceutically acceptable excipient, wherein the composition is in particulate form having a mean particle size in a range of from 2 to 50 μm, and wherein the one or more proteins comprise a spike protein of a coronavirus of SARS, MERS or COVID-19 or variants thereof, and wherein the composition comprises one or more saccharide and a TLR agonist. In embodiments, the TLR agonist is a TLR3 agonist.

In another aspect, the invention relates to a composition for use in preventing infection of respiratory bacteria or viruses (e.g.) coronaviruses by intra-nasal or pulmonary administration of the composition to a subject. Most current vaccine strategies target compositions that provide within whole inactivated or attenuated viruses or bacteria, with the central aim of priming neutralising antibody production. Antigens derived from viruses or bacteria may also be use.

Typically, the composition comprises a relevant antigen, or inactivated or attenuated bacteria or virus such as, e.g. Streptococcus pneumonia, Haemophilus influenza, Moraxella catarrhlis etc. or e.g. a spike protein of a coronavirus of SARS, MERS or COVID-19 or variants thereof, and wherein the composition comprises one or more saccharide and a TLR agonist. A suitable composition is the vaccine composition mentioned above and claimed in the appended claims.

In another aspect, the invention provides antigens based on the spike protein of COVID-19. These proteins include SEQ ID NO:3 and SEQ ID NO:7 or proteins having 99% sequence identity of SEQ ID NO:3 or SEQ ID NO:7. The use of such antigens in vaccination and in vaccination compositions are also aspects of the invention.

The homology between two amino acid sequences or between two nucleic acid sequences is described by the parameter “identity”. Alignments of sequences and calculation of homology scores may be done using e.g., a full Smith-Waterman alignment, useful for both protein and DNA alignments. The default scoring matrices BLOSUM50 and the identity matrix are used for protein and DNA alignments respectively. The penalty for the first residue in a gap is −12 for proteins and −16 for DNA, while the penalty for additional residues in a gap is −2 for proteins and −4 for DNA. Alignment may be made with the FASTA package version v20u6. Multiple alignments of protein sequences may be made using “ClustalW”. Multiple alignments of DNA sequences may be done using the protein alignment as a template, replacing the amino acids with the corresponding codon from the DNA sequence. Alternatively, different software can be used for aligning amino acid sequences and DNA sequences. The alignment of two amino acid sequences is e.g., determined by using the Needle program from the EMBOSS package (http://emboss.org) version 2.8.0. The substitution matrix used is BLOSUM62, gap opening penalty is 10, and gap extension penalty is 0.5.

It is believed that the pulmonal and intranasal administration promotes immunoglobulin switch towards IgA, the immunoglobulin specialized for mucosal surfaces including the lung and gut. The TLR agonist is believed to promote activation of macrophages resulting in increased antigen presenting capacity, increased expression of costimulatory molecules including CD86 in addition to the increased production and release of cytokines and chemokines including interferons. Thus, the TLR2 agonist promotes T cell activation, the foundation for the successful induction of a productive neutralizing B cell response.

The invention also relates to a carrier composition in particulate form for use as carrier for antigenic material derived from respiratory viruses or respiratory bacteria, wherein the carrier composition comprises one or more pharmaceutically acceptable excipients and an adjuvant, wherein the one or more pharmaceutically acceptable excipients comprises i) a disaccharide selected from trehalose, sucrose, and lactose, ii) a polysaccharide selected from cyclodextrins, and wherein the adjuvant is a TLR agonist.

The vaccine composition (i.e. a composition comprising the carrier composition and the antigenic material) is suitable for use by nasal or pulmonary administration.

In preferred aspects, the antigenic material derived from viruses.

In such carrier compositions it is preferred that the disaccharide is trehalose, the polysaccharide is hydroxypropyl-beta-cyclodextrin, and the TLR agonist is a TLR3 agonist.

Trehalose is a stabilizer and a filler and hydroxypropyl-beta-cyclodextrin is a filler or bulk material.

In a carrier composition the concentration of the disaccharide is in a range of from 10% to 60% w/w such as from 30% to 55% w/w or from 40% to 50% w/w, and/or the concentration of the polysaccharide is in a range of from 10% to 60% w/w such as from 30% to 55% w/w or from 40% to 50% w/w, and/or the concentration of the adjuvant is from 0.1% to 5% w/w.

An aspect of the invention relates to a vaccine composition for use in preventing infection of respiratory viruses or bacteria, the composition comprising i) a carrier composition as defined in any one of claims 1-6, and ii) an antigenic material derived from respiratory virus or bacteria.

In the present context the term “antigenic material derived from respiratory viruses or bacteria” is to be understood as e.g. inactivated or attenuated respiratory viruses or bacteria, proteins or fragments thereof isolated from respiratory viruses or bacteria, recombinant proteins or fragments from respiratory viruses or bacteria, or proteins or fragments thereof expresses on the surface of the virus.

The invention also provides vaccine kits comprising a vaccine composition of the invention together with compositions comprising vitamin A and/or vitamin D.

It should be noted that when details are described herein relating to one of the aspects of the invention, such details are also part of the other aspects of the invention. For example, when details relating to a vaccine composition of the present invention are described, these details are also relevant for compositions that are used according to the invention etc.

### DETAILED DESCRIPTION OF THE INVENTION

The objective problem of the present invention may be formulated as providing a vaccine based on a pharmaceutical composition that can be administered intranasally or pulmonary via inhalation to prevent infection of respiratory bacteria and respiratory virus. Non-limiting examples of such viruses are as mentioned herein including SARS, MERS or COVID, especially COVID infections. As showed herein the vaccine composition of the present invention can provide protection with just intra-nasal administration. Thus, the present inventors have found that intranasal and intratracheal (simulating administration to the lungs) administration of a vaccine composition of the present invention provide i) suitable immunization (see FIG. 29), ii) lung protection via formation of neutralizing antibodies (FIG. 30), iii) inhibit virus so it cannot bind to its receptor (FIG. 31), and iv) avoid inflammation such as inflammation in the brain (FIG. 32).

Surprisingly, the present vaccine composition provide protection against infection without a first intra-muscular administration. Thus, the composition of the present invention can be administered without the need of a medical professional to inject it with a syringe in a subject in need thereof. Also surprisingly, the common side-effects observed after administration of a known vaccine compositions (e.g. COVID), namely inflammation in the brain, seem to be totally avoided by the use of nasal administration of the vaccine composition according to the present invention.

In a first aspect the present invention provides a vaccine composition. The physical appearance of the vaccine composition is in the form of a dry powder, notably in particulate form, and the vaccine composition comprises an antigen, an adjuvant and at least one pharmaceutically acceptable excipient. The antigen is a protein expressed on the surface of a respiratory virus or bacteria. The vaccine composition is intended for intratracheal, pulmonal or intranasal administration.

Administration of the vaccine composition may be combined with administration of Vitamin A and/or vitamin D, that may be administered before, at the same time or after the vaccine composition is administered. As mentioned below, the antigen is present in the vaccine composition of the invention in its correct conformation and three-dimensional structure, which ensure correct recognition by B lymphocytes.

The antigen is one or more proteins expressed on the surface of the virus or bacterial causing a respiratory disease. The protein may be the full-length protein or fragments thereof. In particular, the antigen is the full-length protein such as the spike protein of COVID-19 or variants thereof, notably the full-length protein (SEQ ID NO:1), the protein expressed by SARS-CoV-2 Spike S1 (SEQ ID NO:2), ISR52 (SEQ ID NO:3) ISR52-DS (SEQ ID NO: 7) and other known and unknown variants including those mentioned in the examples herein. The composition and/or administration method provides the antigen in its natural conformation allowing for presentation of the protein in its correct 3-dimensional structure with intact binding properties for docking to its natural receptor, i.e., the respiratory tract expressed Ace2 receptor. The intact conformational structure is critical for the initial recognition by naïve IgM B lymphocytes, that after internalization, processing and activation of CD4⁺ T-helper cells are induced to undergo somatic hypermutation and switching to IgG and IgA classes, binding to important interactive parts of the protein such as the receptor binding domain (RBD). The present invention provides a vaccine composition in which the protein is kept in its correctly three-dimensional structure.

The respiratory virus may be influenza virus, respiratory syncytial virus, parainfluenza virus, metapneumovirus, rhinovirus, coronavirus (e.g. SARS, MERS, COVID-19, alpha-, beta-, delta-, gamma-coronavirus, as well as coronaviruses that causes upper respiratory illness (29E, OC43, NL63 and HKU1), adenovirus, bocavirus.

The respiratory bacteria may be Streptococcus pneumoniae, Mycoplasma pneumonia, Haemophilus influenza, Chlamydophilia pneumoniae, Chlamydia Psittaci, Moraxella catarrhalis, Mycobacterium tuberculosis, Mycobacterium avis, Mycobacterium marinum.

The composition of the invention is especially developed when the antigen is a spike protein of COVID-19 selected from SEQ ID NO:3 or SEQ ID NO:7 or spike proteins of COVID-19 having 99% or more sequence identity with SEQ ID NO:3 or SEQ ID NO:7.

As seen from the Examples herein, an aspect of the present invention relates to the development and testing of SARS-CoV-2 Spike S1 protein vaccine candidate, ISR52. The SARS-CoV-2 Spike glycoprotein is suggested to be an essential antigen for protective immunity against severe COVID-19 disease. Several Spike-based subunit vaccines have shown efficacy in clinical and preclinical development. The effect of administering ISR52 via the subcutaneous, intranasal, and intratracheal route is compared, the intratracheal route is a simulation for pulmonary inhalation, which to our knowledge has not previously been tested for SARS-CoV-2 vaccines.

Both respiratory routes gave 100% protection in a lethal challenge mouse model even at a low dose of ISR52, whereas only partial protection was seen with low-dose subcutaneous immunisation. The results showed anti-Spike IgG and IgA that cross reacts with variants of concern, the induction of durable cellular immunity, and demonstrate the robustness of platform when combining the Spike S1 protein with the adjuvant Poly IC:LC.

However, it is contemplated that the ingredients in the vaccine composition, i.e the TLR receptor together with the pharmaceutically acceptable excipients, form a suitable basis formulation for other antigens so that the formulation may be used generally in developing vaccines for nasal administration to prevent infections caused by respiratory bacteria and viruses.

The adjuvant may be a pattern recognition receptor (PRR) agonist such as Toll-like receptor (TLR) agonist or a RIG receptor agonist.

IgA gets produced by class switching of Ig, which is regulated by various processes. The binding of CD40-CD40L and secretion of other cytokines IL-4, IL-5, IL-6, IL-10, and IL-21 promote maturation of Th2 cells, which promote class switching to different Ig subtypes. Retinoic acid, a metabolite of vitamin A, synergistically acts with IL-5 and IL-6 to induce IgA secretion as well.

It is believed that the pulmonal or intranasal administration promotes immunoglobulin switch towards IgA, the immunoglobulin specialized for mucosal surfaces including the lung and gut. The TLR agonist is believed to promote activation of macrophages resulting in increased antigen presenting capacity, increased expression of costimulatory molecules including CD86 in addition to the increased production and release of cytokines and chemokines including interferons. Thus, the TLR agonist promotes T cell activation, the foundation for the successful induction of a productive neutralizing B cell response. TLR3 also activated RIG-1, which stimulated interferon-alpha production. Death or severe disease is observed if Interferon-alpha is produced late or if it is absent in Covid-19 disease.

Mass vaccination campaigns have changed the course of the COVID-19 pandemic and reduced the morbidity and mortality associated with SARS-CoV-2 infection. Nevertheless, waning immunity and antigenic drift will likely see the need for updated and improved vaccines that are distributed equitably across nations. Several second-generation vaccines seek to target the site of infection, namely the respiratory tract, to induce local immunity. These include licensed vaccines reformulated as nasal sprays, and new candidates designed for intranasal administration or aerosolisation (Clinical trial information compiled by WHO COVID-19 vaccine tracker and landscape (who.int). Preclinical results for these candidates suggest comparable or better protection against SARS-CoV-2, with one even describing the induction of sterilizing immunity.

Although needle-based vaccination has been the gold standard for vaccine administration, it poses several limitations. Firstly, injectable vaccines are either formulated as unstable liquids that require cold storage or as lyophilized powders for reconstitution. Secondly, it requires trained health-care personnel, which can be a problem specifically in non-industrialized countries and remote areas. Thirdly, there is a risk of needle-stick injuries and needle re-use, increasing the probability of cross contamination. Fourthly, compliance to needle-based vaccination may be low because of associated needle-phobia and pain at the injection site, and lastly, vaccination by injection predominantly induces systemic immune responses, that are not specifically directed at the pathogen's region of infection, such as mucosal sites. Therefore, alternative ways of administration are highly desirable.

Various needle-free administration routes have been explored. Especially, administration to the respiratory tract seems to be an interesting way of administration, especially for vaccines against airborne transmissible micro-organisms that cause respiratory tract infections. Because of its large surface area, its permeable epithelium, and its highly perfused nature, the respiratory tract mucosa is one of the most optimal targets for the uptake of biopharmaceuticals. Moreover, as the respiratory tract continuously is exposed to foreign materials, the airways contain antigen-presenting cells (APCs), such as alveolar macrophages and dendritic cells, which serves as a defense system against antigens that enter the body.

It is therefore contemplated that directly targeting and delivering a vaccine composition against diseases caused by respiratory infections to the respiratory tract (such as those mentioned herein a COVID-19 vaccine composition), will be advantageous compared with e.g., subcutaneous or intramuscular administered vaccine composition.

Compositions for nasal or pulmonary vaccination are known. However, most of such compositions are in liquid form and intended to be delivered by spray or aerosol. A composition of the present invention is in dry powder form containing particles of the antigen in admixture with one or more pharmaceutically acceptable excipients.

The powder or particulate material is provided to the subject in the form of a suitable device containing the vaccine composition. When the composition is for nasal or pulmonal administration, it is possible to administer the composition by the subject/her(him)self. In contrast to known compositions for nasal or pulmonal administration, a vaccine composition of the present invention is designed to be sniffed up in the nasal cavity by the subject. Dependent on the characteristics of a vaccine composition of the invention the composition can also be applied to the lungs or to the trachea.

Nasal administration is typically via a suitable device. Suitable devices include unit-dose dispensers such as ICOone® provided by ICONovo AB, Lund, Sweden. Powdair from Hovione, Portugal (oral inhalation, but can be adapted to nasal administration. Aptar Unidose—dry powder inhaler for nasal use for pediatric use. Suitable material for such devices includes polypropylene and the like.

Administration to trachea or to the lungs is typically via an inhaler by oral administration.

As mentioned above, the formulation of the vaccine composition is designed in such a manner that the three-dimensional structure of the antigen is maintained. Therefore, the various excipients used in the composition must be selected carefully to avoid destruction of the three-dimensional structure of the antigen.

In this manner it is possible i) to provide a dry vaccine composition with excellent storage stability, ii) to avoid the use of liquids that may give adverse reactions on the application site or that may destroy the three-dimensional structure of the protein, and iii) to ensure that the composition stays on the site of administration for a sufficient period of time.

On top of the potential benefits to immunity, inhaled or nasally administered vaccine formulations have logistical advantages compared to injectable formulations. This is particularly true when the vaccine can be stored and administered (inhaled or nasally administered) as a dry powder, completely avoiding the need for cold chain distribution. Such vaccines could be administered via a disposable dry powder inhaler directly into the lower respiratory tract and are therefore needle-free. Preclinical studies using dry powder vaccines have shown promise for various pathogens and toxins including Mycobacterium tuberculosis, Human papillomavirus, botulinum neurotoxin A, and influenza A virus and a safe and effective SARS-CoV-2 dry powder vaccine would be a boon to global vaccination efforts.

The present invention provides a vaccine composition comprising SARS-CoV-2 spike protein and one or more pharmaceutically acceptable excipient, wherein the composition is in particulate form having a mean particle size in a range of from 1 to 50 μm. The mean particle size depends on where the powder should be located after administration. In the case of administration to the nasal mucosa, the mean particle size should be in a range of from 20 to 50 μm such as from 30 to 40 μm and with a size distribution revealing less than 10% of the particles have a particle size of 10 μm or less. If the vaccine composition is for administration to the lungs, the mean particle size should be 10 μm or less such as at the most 8 μm, at the most 6 μm, at the most 5 μm or in a range of from 1 μm to 5 μm such as in a range of from 3 μm to 5 μm.

The particle size may typically be achieved by using micronized material in dry form, or by micronizing the final composition.

As the vaccine composition is presented in the form of a dry powder, the powder must be filled into a suitable device. Such a device must be made of a material to which the vaccine composition does not adhere as it would lead to variation in the dose administered. Moreover, the vaccine composition must be flowable to efficiently fill the device with the powder and to ensure the correct dosage. It is therefore a requirement that the vaccine composition of the invention has a suitable flowability when measured according to the method described in 2.9.16 of Ph.Eur. 10.0 using a funnel without stem and nozzle 1 with a diameter of 10±0.01 mm.

As seen from the Example herein only 15 mg of the antigen is contained in a total of 5 g of the vaccine composition. Therefore, it is important to choose the one or more pharmaceutically acceptable excipient with a view to i) ensuring the correct conformation of the protein is present, ii) the provision of sufficient flowability to the final vaccine composition, iii) the provision of substances that are not irritating for the nasal or pulmonal mucosa, iv) the provision that they are biodegradable—at least for pulmonal administration, and v) the provision that they do not impart adherence to the device (to exclude incorrect dosing).

Suitable pharmaceutically acceptable excipient is selected from cellulose, cellulose derivatives, methylcellulose, hydroxypropyl cellulose, hydroxypropyl methyl cellulose, microcrystalline cellulose, saccharides including monosaccharides, disaccharides, oligosaccharides, polysaccharides, amino acids including peptides, and mixtures thereof.

In general, a vaccine composition of the invention contains excipients that ensure i) a suitable flowability of the particles/powder, ii) a reduction in intra-particle cohesivity, iii) a stabilization of the protein to maintain the correct conformation, iv) a stabilization of the protein to avoid agglomeration, and v) a suitable volume of the composition to enable handling and processing of the composition.

Amino acids may be present as a surface modifying agent to improve particle flow and reduce intra-particle cohesivity. Other suitable excipients that could be alternately used would include any phospholipid or surfactant, preferably leucine (including di-leucine or tri-leucine), and/or lecithin, NaCl, MgCl₂. Alternately magnesium stearate or sodium stearyl fumarate will achieve the same effect, but requires subsequent mechanical addition rather directly than via spray drying.

A sugar or alternative agent is present to stabilise the protein preventing agglomeration and degradation by fixing and maintaining its physical conformation in an amorphous lattice. This can be achieved by a variety of sugars or sugar alcohols. Preferably disaccharides are used, and preferably trehalose. Sugars with high transition glass temperatures are preferred as this limits mobility at higher ambient moisture levels. Glucose, sucrose, lactose, dextrose, mannitol, maltitol might be considered. Amino acids and amino acid derivates could also be considered.

Additionally, bulking agents are used as process enhancer due to their high viscosity when in solution of the spray dried feedstock. Such excipients include: sugars, sugar alcohols, cyclodextrins, amino acids, polymers or salts thereof. Specific examples are cyclodextrin, hydroxypropyl methylcellulose (HPMC), HPMC derivatives, hydroxypropyl cellulose, lactose, mannitol, polyethylene glycol, polylactic acid (PLA), polylactic-glycolic acid (PLGA) and polysorbate.

In particular, the one or more pharmaceutically acceptable excipient is selected from disaccharides, oligosaccharides, amino acids, peptides and polypeptides.

Monosaccharides are selected from mannose, galactose, fucose, glucose, lactulose.

The disaccharides are selected from trehalose, sucrose, lactose. As seen from the Example, trehalose is a suitable excipient.

Another suitable excipient is a cyclodextrin such as e.g., as hydroxypropyl-beta-cyclodextrin.

A further suitable pharmaceutically acceptable excipient may be an amino acid is selected from leucine or lysine and/or may be selected from di-leucine or tri-leucine. Especially, in relation to up-scaling of the production of a vaccine composition, HPMC seems to be a suitable pharmaceutically acceptable excipient.

An object of the invention is to provide a vaccine composition with as few as possible pharmaceutically acceptable excipients, but still with an aim of keeping the correct three-dimensional structure of the antigen. Thus, in interesting embodiments, apart from the antigen (and the ingredients contained in the antigen composition) and apart from the adjuvant (and the ingredients contained in the adjuvant composition) the vaccine composition contains i) a carbohydrate (mono-, di-, oligo, or polysaccharide), ii) an amino acid, or iii) a peptide. Notably such a vaccine composition contains i) trehalose, ii) hydroxypropyl beta-cyclodextrin, iv) leucine, v) tri-leucine, vi) a combination of trehalose and hydroxypropyl beta-cyclodextrin, vii) a combination of trehalose and leucine, vii) a combination of trehalose and tri-leucine, or viii) a combination of trehalose, hydroxypropyl beta-cyclodextrin and leucine, or ix) a combination of trehalose and tri-leucine, or x) a combination of trehalose, hydroxypropyl beta-cyclodextrin and tri-leucine. HPMC may be included in anyone of i) to x).

In general, a vaccine composition of the invention comprises:
i) an antigen, and
ii) an adjuvant, and/or
iii) a flow-improving agent, and/or
iv) a stabilizing agent for the conformation of the antigen, and/or
v) a bulking agent.

One pharmaceutically acceptable excipient may have one or more of the properties iii)-v) mentioned above.

The antigen is typically present in the composition in a concentration of from 10% to 30% w/w such as in a range of from 10% to 20% w/w such as about 15% w/w.

The adjuvant is typically present in the composition in a concentration of from 0.1% to 5% w/w such as in a range of from 0.1 to 2% w/w, from 0.5% to 1% w/w such as about 0.75% w/w.

The flow-improving agent is typically present in the composition in a concentration of from 2% to 30% w/w such as from 2% to 20%, from 3% to 10% or from 5% to 10% w/w.

The stabilizing agent is typically present in the composition in a concentration of from 2% to 70% w/w such as from 10% to 60% w/w, from 15% to 55% w/w, from 20% to 50% w/w, from 30% w/w to 50% w/w, from 40% to 50% w/w such as about 46-47% w/w.

The bulking agent is typically present in the composition in a concentration of from 2% to 70% w/w such as from 10% to 60% w/w, from 15% to 55% w/w, from 20% to 50% w/w, from 30% w/w to 50% w/w, from 40% to 50% w/w such as about 46-47% w/w.

A vaccine composition of the present invention typically contains leucine (as a flow-improving agent in a concentration as mentioned above such as about 7% w/w), trehalose (as a stabilizing agent in a concentration as mentioned above such as about 46.5% w/w), and cyclodextrin or a derivative thereof (as a bulking agent in a concentration as mentioned above such as about 46.5% w/w). The composition further contains an adjuvant (typically Poly IC:LC) and an antigen.

As mentioned above, a vaccine composition of the present invention also contains an adjuvant. Suitable examples are TLR agonists such as those discussed herein under the heading “Combination with TLR agonists”. Other suitable adjuvants include RIG-1 and MDA5. RIG-1 is a cytosolic pattern recognition receptor responsible for type-1 interferon (IFN1) response. RIG-1 is an essential molecule in the innate immune system for recognizing cells that have been infected with a virus. These viruses include respiratory viruses like influenza A virus and coronaviruses. MDA5 (melanoma differentiation-associated protein 5) is a RIG-1-like receptor dsRNA helicase enzyme that is encoded by the IFIH1 gene in humans. MDA5 is part of the RIG-1-like receptor (RLR) family, which also includes RIG-1 and LGP2, and functions as a pattern recognition receptor capable of detecting viruses.

In embodiments, the TLR agonist is a TLR2 agonist and/or TLR3 agonist such as a TLR3 agonist.

Suitable examples of TLR3 agonists are selected from Poly IC and Poly IC:LC.

The adjuvant such as Poly IC or Poly IC:LC is present in the composition in a concentration corresponding to from 0.01% to 1% w/w such as in a range of from 0.075% to 0.75% w/w, in a range of from 0.08% to 0.5% w/w, from 0.1% to 0.4% w/w, from 0.1% to 0.25% w/w such as about 0.15% w/w based on the total weight of dry matter in the composition.

As mentioned above, a vaccine composition of the present invention may also comprise vitamin A and/or vitamin D (see discussion under the heading “Combination with vitamin A” and the heading “Combination with vitamin D”)

### Coronaviruses

The vaccine composition of the present invention is suitable for use in vaccination against coronaviral disease such as e.g., SARS-CoV2.

Other coronaviruses of interest are e.g. SARS, MERS, COVID-19, alpha-, beta-, delta-, gamma-coronavirus, as well as coronaviruses that causes upper respiratory illness (29E, OC43, NL63 and HKU1)

The vaccine composition of the invention comprises antigen. The antigen may be a protein or a multimer thereof, a peptide or a multimer thereof or an attenuated virus or bacterium. In particular, the antigen is the full-length protein expressed on the surface of the virus or bacterium. In embodiments the antigen may be the spike protein of SARS-CoV-2 or a variant thereof or it may be attenuated SARS-CoV-2 or a component thereof, or the spike protein from SARS-Cov-2 or a part thereof such a SARS-CoV-2 Spike S1 subunit including the RBD.

The Spike dosage used in preclinical mouse studies have ranged between 5-80 μg. In clinic it is planned to evaluate 10-120μ such as 10 μg, 50 μg and 120 μg of the spike protein. The dosage of the adjuvant is typically from 10 μg to 120 μg. Poly IC:LC have been used in the 3-60 μg span in the preclinical rodent studies. For the clinical trial we are planning to use 10-60 μg such as 20 μg or 40 μg.

Oncovir who produces the Poly IC:LC have used it in clinical trials for nasal dosage up to 1 mg without adverse effects.

Vitamin A and D have been sparsely evaluated, in the mouse studies we have used 200 ng Calcitrol per mouse and 40 μg ATRA (all-trans retinoic acid) per mouse.

Coronaviruses are a group of related RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause SARS, MERS, and COVID-19.

Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives. Coronaviruses are large, roughly spherical particles with unique surface projections. Their size is highly variable and generally is an average diameter of 120 nm. Extreme sizes are known from 50 to 200 nm in diameter. The total molecular weight is on average 40,000 kDa They are enclosed in an envelope embedded with a number of protein molecules. The lipid bilayer envelope, membrane proteins, and nucleocapsid protect the virus when it is outside the host cell.

Coronavirus disease 2019 (COVID-19) is a contagious respiratory and vascular disease caused by severe acute respiratory syndrome coronavirus 2 (SARS-CoV-2).

Common symptoms of COVID-19 include fever, cough, fatigue, breathing difficulties, and loss of smell and taste. Symptoms begin one to fourteen days after exposure to the virus. While most people have mild symptoms, some people develop acute respiratory distress syndrome (ARDS). ARDS can be precipitated by cytokine storms, multi-organ failure, septic shock and blood clots Longer-term damage to organs (in particular, the lungs and heart) has been observed.

COVID-19 spreads via a number of means, primarily involving saliva and other bodily fluids and excretions. These fluids can form small droplets and aerosols, which can spread as an infected person breathes, coughs, sneezes, sings, or speaks. This is suspected to be the main mode of transmission. The virus may also spread via contaminated surfaces and direct contact. Infection mainly happens when people are near each other long enough. It can spread as early as two days before infected persons show symptoms (presymptomatic), and from asymptomatic (no symptoms) individuals. People remain infectious for up to ten days in moderate cases, and two weeks in severe cases.

Side-effects and/or late effects include one or more of tiredness, less energy, muscle pain, joint pain, sore joints and bones etc.

The vaccine composition of the present invention may be used in combination with e.g. vitamin A and/or vitamin D.

The respiratory virus may be influenza virus, respiratory syncytial virus, parainfluenza virus, metapneumovirus, rhinovirus, coronavirus, adenovirus, bocavirus.

The respiratory bacteria may be Streptococcus pneumoniae, Mycoplasma pneumonia, Haemophilus influenza, Chlamydophilia pneumoniae, Chlamydia Psittaci, Moraxella catarrhalis, Mycobacterium tuberculosis, Mycobacterium avis, Mycobacterium marinum.

Combination with a TLR Agonist

Toll-like receptors (TLRs) are a class of proteins that plays an important role in recognition of viral particles and activation of the innate immune system. They are membrane-spanning receptors usually expressed on macrophages and dendritic cells and recognize molecules derived from microbes. Once the microbes have passed e.g. the skin or mucosa they are recognized by TLRs, which activate immune cell responses. The human TLRs include TLR1, TLR2, TLR3, TLR4, TLR5, TLR6, TLR7, TLR8, TLR9, TLR10. Humans lack genes for TLR11, TLR12, TLR13.

TLR1, TLR2, TLR4, TLR5, TLR6 and TLR10 are located on the cell membrane whereas TLR3, TLR7, TLR8 and TLR9 are located in intracellular vesicles.

Activation of TLR pathways leads to secretion of pro-inflammatory cytokines, such as interleukin-1 (IL-1), IL-6, and tumor necrosis factor-α, as well as type 1 interferon. Different TLRs, like TLR2, TLR3, TLR4, TLR6, TLR7, TLR8, and TLR9 are potentially important in COVID-19 infection.

Examples of TLR2 agonists are macrophage-activating lipopeptide (MALP-2), Pam2Cys, PEG-Pam2Cys.

Examples of TLR3 agonists are PIKA analogue of Poly IC), Poly IC (polycytidylic acid), Poly IC:LC (Poly IC condensed with poly-L-lysine and carboxmethylcellulose), and liposome encapsulated Poly IC:LC.

Examples of TLR4 agonists are LPS (lipopolysaccharide), MPL (monophosphoryl lipid A), FimH (fimbria H protein), AGPs (aminoalkyl glucosaminide phosphates)

Examples of TLR5 agonists are bacterial flagella proteins.

An example of a TLR7 agonist is 1-(2-methylpropyl)-1H-imidazo(4,5-c) quinoline-4-amine (Imiquimod).

Examples of TLR9 agonists are CpG and CpG-ODN.

As mentioned above, the combination may also be with other adjuvants such as RIG-1 or MDA5.

Combination with Vitamin A

Immunoglobulin A (IgA), one of the five primary immunoglobulins, plays a pivotal role in mucosal homeostasis in the gastrointestinal, respiratory, and genitourinary tracts, functioning as the dominant antibody of immunity in this role. It is the second most abundant immunoglobulin type found in the body and, consequently, has a crucial role in protection against antigens.

IgA is produced by class switching of Ig, which is regulated by various processes. The binding of CD40-CD40L and secretion of other cytokines IL-4, IL-5, IL-6, IL-10, and IL-21 promote maturation of Th2 cells, which promote class switching to different Ig subtypes. Retinoic acid, a metabolite of vitamin A, synergistically acts with IL-5 and IL-6 to induce IgA secretion as well.

Vitamin A (retinoid) is a micronutrient known to be required in trace amounts in the diet of practically all vertebrate animals, as it cannot be synthesized in sufficient quantities to maintain physiological health. High concentrations can have some therapeutic effects, as the vitamin A and its metabolites are known to have adjuvant activity.

The retinol must be oxidized to retinal by intracellular enzyme alcohol dehydrogenase (ADH) prior to being irreversibly catabolized by retinal dehydrogenase (RALDH) to its biologically active form all-trans-retinoic acid (from now referred to as RA). This bioactive metabolite can be synthesized by many cell types and tissues known to possess the RALDH enzyme necessary for such a conversion, including DCs from different tissues, e.g., gut, lungs, skin and their draining lymph nodes.

Vitamin A was already in the 1980's found to control the transcellular transport of the IgA dimers across the epithelial cells. During the following decades the impact of vitamin A interacting with several immune cells and stromal cells in the lamina propria was further explored.

One special characteristic of mucosal immune cells is their unique mucosal-imprinting phenotype, a property required in subsequent steps in the production and secretion of IgA antibody isotype. This special property appears to require the presence of RA in the mucosal environment. The key finding on the influence of vitamin A (or RA) on the regulation of mucosal immune response was that RA has a central role in differentiation of DCs and that the mucosal DCs could metabolize retinol into retinoic acid.

Another important function of RA is to promote DC-dependent generation of IgA-antibody secreting cells from B cells and this process is enhanced by IgA-inducing cytokines like IL-5/IL-6. In fact, different lines of evidence from several animal models and human studies all agree that the synthesis of RA by lymphoid tissue DCs and other non-immune cells is needed to induce IgA expression in B cells. It is concluded from these studies that RA functions as a specific IgA isotype switching factor that facilitates the differentiation of IgA+ antibody secreting cells and enhances IgA production in the presence of TGF-β. The effectiveness of this action is subjected to modulation by the presence of IL-5 or IL-6 in the microenvironment.

Combination with Vitamin D

In general, vitamin D functions to activate the innate and dampen the adaptive immune systems with antibacterial, antiviral and anti-inflammatory effects. Deficiency has been linked to increased risk or severity of viral infections, including HIV and COVID-19. Low levels of vitamin D appear to be a risk factor for tuberculosis, and historically it was used as a treatment.

Supplementation slightly decreases the risk and severity of acute respiratory tract infections, and also the exacerbation of asthma. There is no evidence for vitamin D affecting respiratory infections in children under five years of age. Vitamin D supplementation substantially reduces the rate of moderate or severe exacerbations of COPD in people with baseline 25(OH)D levels under 25 nmol/L but not in those with less severe deficiency.

Thus, vitamin 0 is a well-known and safe active substance for use in medicine.

Vitamin D is a group of fat-soluble secosteroids known for increasing intestinal absorption of calcium, magnesium, phosphate, and many other biological effects.

Five different vitamin D are known: vitamin D₁ (also known as a 1:1 mixture of ergocalciferol and lumisterol), vitamin D₂ (also known as ergocalciferol, which can be made from ergosterol), vitamin D₃ (also known as cholecalciferol, which can be made from 7-dehydrocholesterol), vitamin D₄ (also known as 22-dihydrocalciferol), and vitamin D₅ (also known as sitocalciferol, which can be made from 7-dehydrositosterol). Vitamin D used in the present invention is selected from vitamin D₁, vitamin D₂, vitamin D₃, vitamin D₄, vitamin D₅ or a mixture thereof.

In humans the most important vitamin D's are vitamin D₃ (cholecalciferol) and vitamin D₂ (ergocalciferol). These are known collectively as calciferol. Vitamin D₂ was chemically characterized in 1931. In 1935, the chemical structure of vitamin D₃ was established and proven to result from the ultraviolet irradiation of 7-dehydrocholesterol.

Chemically, the various forms of vitamin D are secosteroids, i.e., steroids in which one of the bonds in the steroid rings is broken. The structural difference between vitamin D₂ and vitamin D₃ is the side chain of D₂ that contains a double bond between carbons 22 and 23, and a methyl group on carbon 24.

Accordingly, vitamin D₂ and D₃ are especially preferred in a composition of the invention. Most preferred is vitamin D₃.

The major natural source of the vitamin is synthesis of cholecalciferol in the lower layers of skin epidermis through a chemical reaction that is dependent on sun exposure (specifically UVB radiation). Cholecalciferol and ergocalciferol can be ingested from the diet and from supplements. Only a few foods, such as the flesh of fatty fish, naturally contain significant amounts of vitamin D. In the U.S. and other countries, cow's milk and plant-derived milk substitutes are fortified with vitamin D, as are many breakfast cereals. Mushrooms exposed to ultraviolet light contribute useful amounts of vitamin D. Dietary recommendations typically assume that all of a person's vitamin D is taken by mouth, as sun exposure in the population is variable and recommendations about the amount of sun exposure that is safe are uncertain in view of the skin cancer risk.

Vitamin D from the diet, or from skin synthesis, is biologically inactive. It is activated by two protein enzyme hydroxylation steps, the first in the liver and the second in the kidneys. As vitamin D can be synthesized in adequate amounts by most mammals if exposed to sufficient sunlight, it is not essential, so technically not a vitamin. Instead it can be considered a hormone, with activation of the vitamin D pro-hormone resulting in the active form, calcitriol, which then produces effects via a nuclear receptor in multiple locations.

Cholecalciferol is converted in the liver to calcifediol (25-hydroxycholecalciferol); ergocalciferol is converted to 25-hydroxyergocalciferol. These two vitamin D metabolites (called 25-hydroxyvitamin D or 25(OH)D) are measured in serum to determine a person's vitamin D status. Calcifediol is further hydroxylated by the kidneys to form calcitriol (also known as 1,25-dihydroxycholecalciferol), the biologically active form of vitamin D. Calcitriol circulates as a hormone in the blood, having a major role regulating the concentration of calcium and phosphate, and promoting the healthy growth and remodeling of bone. Calcitriol also has other effects, including some on cell growth, neuromuscular and immune functions, and reduction of inflammation.

Vitamin D has a significant role in calcium homeostasis and metabolism. Its discovery was due to effort to find the dietary substance lacking in children with rickets (the childhood form of osteomalacia). Vitamin D supplements are given to treat or to prevent osteomalacia and rickets. The evidence for other health effects of vitamin D supplementation in the general population is inconsistent. The effect of vitamin D supplementation on mortality is not clear, with one meta-analysis finding a small decrease in mortality in elderly people, and another concluding no clear justification exists for recommending supplementation for preventing many diseases, and that further research of similar design is not needed in these areas.

The active vitamin D metabolite calcitriol mediates its biological effects by binding to the vitamin D receptor (VDR), which is principally located in the nuclei of target cells. The binding of calcitriol to the VDR allows the VDR to act as a transcription factor* that modulates the gene expression of transport proteins (such as TRPV6 and calbindin), which are involved in calcium absorption in the intestine. The vitamin D receptor belongs to the nuclear receptor superfamily of steroid/thyroid hormone receptors, and VDRs are expressed by cells in most organs, including the brain, heart, skin, gonads, prostate, and breast.

VDR activation in the intestine, bone, kidney, and parathyroid gland cells leads to the maintenance of calcium and phosphorus levels in the blood (with the assistance of parathyroid hormone and calcitonin) and to the maintenance of bone content.

One of the most important roles of vitamin D is to maintain skeletal calcium balance by promoting calcium absorption in the intestines, promoting bone resorption by increasing osteoclast number, maintaining calcium and phosphate levels for bone formation, and allowing proper functioning of parathyroid hormone to maintain serum calcium levels. Vitamin D deficiency can result in lower bone mineral density and an increased risk of reduced bone density (osteoporosis) or bone fracture because a lack of vitamin D alters mineral metabolism in the body. Thus, vitamin D is also critical for bone remodeling through its role as a potent stimulator of bone resorption.

The VDR regulates cell proliferation and differentiation. Vitamin D also affects the Immune system, and VDRs are expressed in several white blood cells, including monocytes and activated T and B. In vitro, vitamin D increases expression of the tyrosine hydroxylase gene in adrenal medullary cells, and affects the synthesis of neurotrophic factors, nitric oxide synthase and glutathione.

Vitamin D receptor expression decreases with age and findings suggest that vitamin D is directly related to muscle strength, mass and function, all being important factors to an athlet's performance.

### Combination Vaccines

A vaccine composition of the present invention may also contain two or more antigens such a mix of protein antigens expressed on viruses' surfaces. Such a mix could be spike from a variant of SAR-CoV-2 with hemaglutinins and/or neuroaminidases from Influenza A and/or B, respectively to obtain a double or triple combo vaccine composition.

### Other Aspects of the Invention

The present invention also relates to
i) a vaccine kit comprising:
   a composition comprising an antigen, a TLR agonist and one or more pharmaceutically acceptable excipients, and
   a label informing that said composition is to be used for vaccination by co-administration of vitamin A,
ii) i) a vaccine kit comprising:
   a composition comprising an antigen, a TLR agonist and one or more pharmaceutically acceptable excipients, and
   a label informing that said composition is to be used for vaccination by co-administration of vitamin D,
iii) i) a vaccine kit comprising:
   a composition comprising an antigen, a TLR agonist and one or more pharmaceutically acceptable excipients, and
   a label informing that said composition is to be used for vaccination by co-administration of vitamin A and vitamin D,
iv) a vaccine kit comprising:
   a first composition comprising an antigen, a TLR2 agonist and one or more pharmaceutically acceptable excipients, and
   a second composition comprising vitamin A,
v) a vaccine kit comprising:
   a first composition comprising an antigen, a TLR2 agonist and one or more pharmaceutically acceptable excipients, and
   a second composition comprising vitamin D,
vi) a vaccine kit comprising:
   a first composition comprising an antigen, a TLR2 agonist and one or more pharmaceutically acceptable excipients, and
   a second composition comprising vitamin A,
   a third composition comprising vitamin D.

### General Use of the Vaccines of the Invention

The vaccinations methods and the vaccine compositions of the invention disclosed herein may be used to provide individuals with immunity against bacterial or viral agents, and in particular against respiratory bacteria or viruses as described herein.

### Pharmaceutical Compositions for Use in Vaccination Against Diseases Caused by Coronaviruses

The present invention also provides vaccination kits comprising a pharmaceutical composition comprising the antigen and a TLR3 agonist together with one or more pharmaceutically acceptable excipients. The present invention also relates to pulmonal or intranasal compositions comprising the antigen and a TLR3 agonist together with at least one pharmaceutically acceptable excipient Pharmaceutical compositions for nasal, intratracheal and pulmonary administration are in solid form. As seen from the example herein, the solid form may be obtained by drying of a liquid formulation. The powder is formulated for administration by sniffing but may also be in the form of a powder spray or a dry powder inhaler.

The vaccination method may consist of a single administration or a plurality of administrations over a period of time. In particular, the oral administration of vitamin A and/or vitamin D may consist of a plurality of administrations.

The composition may conveniently be presented in a suitable dosage such as a unit dosage form and may be prepared by any of the methods well known in the art of pharmacy. Such methods include the step of bringing into association the active ingredient (antigen) and the TLR3 agonist with one or more excipients. In general, the formulations are prepared by uniformly and intimately bringing into association the active ingredient with liquid carriers or finely divided solid carriers or both.

Depending upon the particular vaccination and the individual to be vaccinated, as well as the route of administration, the compositions may be administered at varying doses and/or frequencies.

The pharmaceutical compositions must be stable under the conditions of manufacture and storage; thus, if necessary, they should be preserved against the contaminating action of microorganisms such as bacteria and fungi. In case of liquid formulations as intermediate formulations such as solutions, dispersion and suspensions, the carrier can be a solvent or dispersion medium containing, for example, water, ethanol, polyol (e.g. glycerol, propylene glycol and liquid polyethylene glycol), vegetable oils, and suitable mixtures thereof. In case of solid formulations, dry powder formulations are usually prepared by mixing the micronized active particles with carrier particles such as those described herein before including HPMC, hydroxypropyl cyclodextrin, sorbitol, lactose, trehalose, leucin or mannitol.

The compositions may also include pH-adjusting agents, stabilizing agents, surfactants, solubilizers (such as triacetin, triethylcitrate, ethyl oleate, ethyl caprylate, dimethylacetamide, N-methylpyrrolidone, N-hydroxyethylpyrrolidone, polyvinylpyrrolidone), absorption promoting agents (such as polyoxyethylene glycol or fatty acid mono- or diglyceride esters), dispersing agents, preservatives etc.

It should be understood that in addition to the ingredients particularly mentioned above the formulations of this invention may include other agents conventional in the art having regard to the type of formulation in question. A person skilled in the art will know how to choose a suitable formulation and how to prepare it (see eg Remington's Pharmaceutical Sciences 18 Ed. or later). A person skilled in the art will also know how to choose a suitable administration route and dosage.

The pharmaceutically acceptable salts of the TLR agonist include conventional salts formed from pharmaceutically acceptable inorganic or organic acids or bases as well as quaternary ammonium acid addition salts. More specific examples of suitable acid salts include hydrochloric, hydrobromic, sulfuric, phosphoric, nitric, perchloric, fumaric, acetic, propionic, succinic, glycolic, formic, lactic, maleic, tartaric, citric, palmoic, malonic, hydroxymaleic, phenylacetic, glutamic, benzoic, salicylic, toluenesulfonic, methanesulfonic, naphthalene-2-sulfonic, benzenesulfonic hydroxynaphthoic, hydroiodic, malic, steroic, tannic and the like. Other acids such as oxalic, while not in themselves pharmaceutically acceptable, may be useful in the preparation of salts useful as intermediates in obtaining the compounds of the invention and their pharmaceutically acceptable salts. More specific examples of suitable basic salts include sodium, lithium, potassium, magnesium, aluminium, calcium, zinc, N,N′-dibenzylethylenediamine, chloroprocaine, choline, diethanolamine, ethylenediamine, N-methylglucamine and procaine salts.

### Results

The suitability as a carrier for a vaccine composition for nasal administration for use in vaccination against a respiratory bacteria or virus, can be seen in the Examples herein. In the present context, a carrier denotes the pharmaceutically acceptable excipients together with a TLR agonist, but does not include any antigen, any inactivated or attenuated virus or bacteria.

In Example 3 is reported that respiratory administration of spike protein vaccine ISR52 protects from lethal challenge (studies AB21-04, AB21-31).

The SARS-CoV-2 Spike glycoprotein has been suggested to be an essential antigen for protective immunity against severe COVID-19 disease. Indeed, several spike-based subunit vaccines have shown efficacy in clinical and preclinical studies WHO vaccine tracker. We therefore selected soluble spike protein, based on the founder SARS-CoV-2 sequence as our first vaccine candidate in the knowledge that dried powder formulations of spike protein could be prepared at a later time.

As described in detail in Example 3, our vaccine candidate (ISR52) was then prepared for administration into female AC70 hACE2 mice, a lethal SARS-CoV and SARS-CoV-2 infection model (Muñoz-Fontela et al., 2020; Tseng et al., 2007; Xu et al., 2021; Yoshikawa et al., 2009). We used a low and high dose of ISR52 with poly I:C and all-trans retinoic acid (ATRA) as adjuvants. We tested three different routes of administration: subcutaneous (s.c.) injection, intranasal (i.n.), and via the intratracheal (i.t.) route. While intranasal immunisation has been previously interrogated for SARS-CoV-2 vaccines, intratracheal administration has not to our knowledge been reported. We immunised 7 or 8 mice per group and included an additional control group of unvaccinated mice. Two immunisations were spaced two weeks apart, and we then challenged with 2×10⁵ TCID50 of founder-type SARS-CoV-2.

After challenge, we monitored the health of all mice each day, and euthanised mice when they had lost >20% of their body weight or displayed stronger symptoms in line with our ethical permit. According to these criteria, all mice from the non-vaccinated control group were euthanised on day 4 post infection. For the low-dose subcutaneous group, 5/7 mice died on day 5 or 6 post infection, indicating suboptimal protection. We found 100% protection in all other groups, including at lower dose for the i.n. and i.t. groups, indicating that ISR52 raises protective immunity against SARS-CoV-2. Mice which survived challenge did not have quantifiable levels of SARS-CoV-2 in BAL, indicating either that they had cleared the infection by day 11 post infection or that the virus never entered the lungs. Histopathology analysis of lung tissue showed the presence of inflammatory cells in the lungs, likely indicating the presence of infection, though we did not see clear differences between groups. Most interestingly, inflammation and necrosis in brain tissue was observed for unvaccinated (8/8) and both low (5/7) and high (2/7) dose subcutaneously vaccinated groups; no similar observations were made for animals vaccinated by the respiratory route. Neuronal damage is a major part of the pathology of SARS-CoV-2 in the hACE2 mouse models, and it was therefore highly relevant that both i.n. and it. administration of ISR52, even at low dose, protected from signs of neuronal damage.

Bronchoalveolar lavage fluid (BAL), and organs were harvested from mice at the day of termination. We analysed the presence of SARS-CoV-2 RNA at the time of euthanasia in BAL and lung tissue (FIG. 29). We detected SARS-CoV-2 RNA in BAL from mice which died during infection, including control mice and 4 mice from the low-dose s.c. group. Meanwhile, low to no detectable viral RNA was found in mice that survived (Ct >35). We obtained similar results with lung tissue, though fewer animals overall had quantifiable SARS-CoV-2 RNA. These results indicate that the vaccinated animals, with the exception of the low dose s.c. group, either cleared the infection by day 11 post infection or that the virus never entered the lungs. We also investigated evidence of pathology in brain tissue of challenged mice, since neuronal damage is a major part of the pathology of SARS-CoV-2 in hACE2 mouse models. Histopathology analysis revealed inflammation and necrosis in brain tissue for unvaccinated (8/8) and both low (5/7) and high (2/7) dose subcutaneously vaccinated groups; no similar observations were made for animals vaccinated by the respiratory routes. These results demonstrates that both i.n. and i.t. administration of ISR52, even at low dose, protect from signs of neuronal damage in the hACE2 mouse model. Overall, we saw a clear benefit of respiratory-tract vaccination versus traditional subcutaneous vaccination at low doses of ISR52.

### ISR52 Immunisation Induces Humoral Responses that Cross React with Variants of Concern (Study AB21-33, AB21-73)—Examples 4 and 6

Since anti-Spike antibodies are likely important in protective immunity against SARS-CoV-2, we furthermore investigated the reactivity of sera from vaccinated mice collected prior to viral challenge. We analysed levels of both serum IgG and serum IgA against spike S1 proteins from the founder strain Wuhan, China), and against the receptor binding domain (RBD) for the alpha variant of concern (VOC) B.1.1.7, and the beta VOC B.1.351. We found that all vaccinated groups, had detectable anti-Spike IgG to at least a 1:10000 dilution for each variant, with higher dose groups having higher levels of IgG. We found also larger differences between the vaccinated groups when we analysed anti-Spike IgA; however, intranasal vaccination gave a stronger serum IgA response compared with intratracheal vaccination, while subcutaneous immunisation delivered a negligible IgA titre. Since one of the aims of intranasal and intratracheal immunisation is to induce a local immune response at the relevant infection site, we also harvested BAL from animals at the time of euthanasia. We analysed the presence of anti-spike IgA in BAL, finding the most robust induction of IgA in the high dose intranasal group, with little induction present in the subcutaneous groups, and a weak induction in the intratracheal groups. This pattern was reproduced when we analysed the neutralising capacity of antibodies present in BAL. We found that subcutaneous immunisation was not associated with neutralising activity in BAL (1 out of 13 animals in both low and high dose groups had detectable titres of neutralising antibodies).

However, we could find that 44% of the intranasal and intratracheal groups had detectable levels of neutralising antibodies up to a titre of 1 in 32 (low dose i.n. 2/8; high dose i.n. 5/7; low dose it. 1/7; high dose i.t. 4/5).

Taken together with survival and brain histopathology data, these data suggest a more robust protection is elicited by i.n. and it. administration compared with s.c. immunisation. These results are consistent with previous studies of intranasal vaccination but show for the first time evidence for the effectiveness of intratracheal immunisation against SARS-CoV-2.

Example 5 relates to dose-dependent response to vaccine candidate ISR52 updated with variant spike (study AB21-31).

Our first challenge study showed the promise of vaccine candidate ISR52 when administered either intranasally or intratracheally for induction of antibodies and protection from SARS-CoV-2 disease. Following changes in predominant circulating variants, and their possible immune escape mutations, we updated our vaccine candidate to include equal amounts of monomeric alpha variant Spike (B.1.1.7) and beta variant Spike (B.1.351). We focused on i.n. an i.t. immunisation, using 5 μg and 20 μg total amount of Spike in this study, compared to 10 μg and 80 μg in the previous study (Table 3). We used the same immunisation and challenge schedule as the previous study (FIG. 2A), immunising 11 female hACE2 mice per group. To challenge the power of our vaccine candidate we used a higher infectious dose of SARS-CoV-2 (1×10⁶ TCID50). As with the previous study, all control unvaccinated mice lost weight, showed other symptoms, and were euthanised at day 4 post infection. Unfortunately, a equipment failure led to the death of four mice in the high-dose intranasal group at 2 days post infection; the deaths of these mice were considered not related to infection and they were therefore excluded from further analysis. The low-dose groups showed a modest effect of the vaccine; 3/11 from the low-dose i.n. group and 4/11 from the low-dose i.t. group survived infection, whereas the remaining animals were euthanised on days 4, 5, or 6. Both of these groups lost, on average, a significant proportion of their body weight by day 4 post infection. The high-dose groups showed a greater level of protection, where 4/7 of the i.n. and 7/11 of the it. groups survived challenge. Consistent with this, we did not observe a significant decline in weight in these mice. There was thus a clear dose-dependency of our vaccine, though we did not see differences between the route of administration by analysing health status and survival. We analysed SARS-CoV-2 RNA in BAL from mice at the time of euthanasia and found detectable levels in the majority of animals that were euthanised before the end of the experiment; the animals who survived were euthanised at 11 days post infection and did not have detectable levels of SARS-CoV-2 RNA. Overall, our modified vaccine candidate showed good effectivity against a very high dose of virus from a different strain than those to which the mice where immunised; this was true for both i.n. and i.t. administration at the higher 20 μg dose.

Pre- and post-challenge antibody responses do not significantly differ between i.n. and i.t. administration (study AB21-33, AB21-34, AB21-74)

During this study, we harvested and analysed serum after the first immunisation, after the second immunisation, and at termination as expected, one immunisation gives a weak anti-Spike IgG response. The second immunisation clearly leads to a strong increase in anti-Spike IgG titre in a dose dependent manner, supporting our two-dose strategy, however we did not see differences here between immunisation route (i.n. vs i.t.). We then looked at cross-reactivity of serum IgG and serum IgA after two immunisations, to four variant spike proteins, from the alpha, beta and delta (B.1.617.2) variants of concern, and the kappa (B.1.617.1) variant under monitoring. Despite immunising with a combination of alpha and beta spike proteins, we saw similar levels of IgG and IgA against all variant spike proteins within groups. Between groups, we saw evidence of dose dependency but again no significant differences between i.n. and i.t. groups of the same dose.

To complement our serological data, we pooled pre-challenge sera from each vaccine group and analysed the neutralising activity of the sera against the beta variant (to which the mice had been specifically immunised) and the delta variant (against which the mice had not been immunised). Once again, we saw a dose-dependent effect of both i.n. and i.t. administration. When comparing the same dose, i.n. and i.t. vaccination had identical neutralising titres against the beta variant, consistent with the serological data against the beta spike protein. Similarly, the high dose i.n. and i.t. groups both had had titres of 1 in 64 against the delta strain. The only potential difference was seen with the low dose groups; low-dose intratracheal had a titre of 1 in 32 against the delta variant, but there was no detectable neutralising antibodies in the low dose intranasal group at a detection limit of 1 in 16. We also analysed neutralisation in BAL against the Founder strain. We found neutralising antibodies in the majority of protected mice, but not in susceptible mice. This strong correlation suggests that the ability of the vaccine to induce an immune response in the airways is important for its ability to protect against severe infection and death. At the same time, despite a high viral infectious dose, we did not see significant differences between intranasal and intratracheal immunisation. This could be a result of a ‘common mucosal immunity’ phenomenon, where immunisation at one mucosal site leads to immunity at distal sites. We now plan to develop our vaccine as a dry powder for inhalation and move forward with Phase I/II clinical trials.

### Dose-Dependent Response to Vaccine Candidate ISR52 Based on Variant Spike

Following changes in predominant circulating variants, and their possible immune escape mutations, we updated our vaccine candidate to include equal amounts of alpha variant and beta variant Spike S1. We used the i.n. and i.t. administration routes and decided to lower the dose to 5 and 20 g of total Spike S1, this time immunising 11 female hACE2 mice per group. During this study, we harvested and analysed serum after the first and second immunisations. As expected, one immunisation gives a weak anti-Spike IgG response. The second immunisation clearly leads to a strong increase in anti-Spike IgG titre in a dose dependent manner, supporting our two-dose strategy; however, no differences between immunisation routes were observed (i.n.vs i.t.). We then investigated cross-reactivity of serum IgG and serum IgA after two immunisations, to three variant Spike RBDs, from the alpha, beta and delta variants of concern. Despite immunising with a combination of alpha and beta Spike S1 proteins, we saw similar levels of IgG and IgA against all variant RBDs within groups (FIG. 3C, S3A, S3B, S3C, S3D, S3E, S3F, S3G). Between groups, we saw evidence of dose dependency but again no significant differences between i.n. and i.t. groups of the same dose.

These serological data were complemented by analysis of neutralizing activity in pooled pre-challenge sera from each group. We found virus neutralization against the beta and the delta variant in a dose dependent manner in vaccinated mice from both i.n. and it. groups. Both higher dose 20 μg i.n. and i.t. groups had neutralizing titres of 1:128 and 1:64 against beta and delta, respectively, consistent with our serological data (FIG. S3B, S3C). The only potential difference was seen with the low dose groups; low-dose it. showed a titre of 1 in 32 against the delta variant, but there was no detectable neutralizing antibodies in the low dose i.n. group at a detection limit of 1 in 16.

Having observed clear pre-challenge immune responses with our updated vaccine, we decided to challenge the power of our vaccine candidate by using a higher infectious dose of SARS-CoV-2 (1×10⁶ TCID50) compared to the first challenge study. As with the first challenge study, all control unvaccinated mice lost weight, demonstrated symptoms, and were euthanized at day 4 post infection. Unfortunately, an equipment failure led to the death of four mice in the high-dose intranasal group at 2 days post infection; the deaths of these mice were considered not related to infection and they were therefore excluded from further analysis. The low-dose groups showed a modest effect of the vaccine; 3/11 from the low-dose i.n. group and 4/11 from the low-dose i.t. group survived infection, whereas the remaining animals were euthanized on days 4, 5, or 6. Both of these groups lost, on average, a significant proportion of their body weight by day 4 post infection. The high-dose groups showed a greater level of protection, where 4/7 of the i.n. and 7/11 of the i.t. groups survived challenge. Consistent with this, we did not observe a significant decline in weight in these mice. There was thus a clear dose-dependency of our vaccine, though we did not see differences between the route of administration by analysing health status and survival.

Finally, we analysed neutralizing activity in BAL harvested at termination. We found neutralizing antibodies in the majority of protected mice, but not in susceptible mice. This strong correlation suggests that the ability of the vaccine to induce an immune response in the airways is important for its ability to protect against severe infection and death. At the same time, despite a high viral infectious dose, we did not see significant differences between intranasal and intratracheal immunisation. This could be a result of a ‘common mucosal immunity’ phenomenon, where immunisation at one mucosal site leads to immunity at distal sites.

### ISR52 Based on Alpha Spike Reveals the Induction of Long-Term T Cell and Broad Cross-Reactive Antibody Responses by Intranasal Administration

Initial results revealed that a vaccine based on solely Spike S1 of the Alpha variant should lead to a broad cross-reactive immune response. Hence, the vaccine was updated to be based on Spike S1 Alpha alone as antigen and evaluated in terms of both antibody and T cell responses. We focused on i.n. and it. immunisation in female C57BL/6J (black 6) mice, using 5 μg and 20 μg total amount of Spike S1 in this study. We immunised 5 or 6 mice per group at day 0 and day 14, and harvested blood, BAL, and splenocytes at day 28. Spike IgG levels in serum were dose dependent and cross-reacted with the RBD of the founding strain, Alpha VOC, Delta VOC (B.1.617.2) and Omicron VOC (B.1.1). We found a similar broad response pattern with BAL IgA, indicating the induction of anti-Spike IgA in a relevant tissue prior to any virus exposure, thus confirming that our immunisation strategy leads to the induction of local immune responses.

T cell responses are likely important in eliciting long-term cross-protection against severe COVID-19 disease. It has also been shown that T cell responses in absence of detectable neutralizing antibodies protect from lethal SARS-CoV-2 challenge in hamster and mouse models. Therefore, we therefore investigated if ISR52 could elicit Spike peptide-responsive T cells. We stimulated T cells from mice two weeks post second immunisation and found both IFN-γ and IL-2 producing T cells by ELISpot in all mice administered i.n., consistent with previous analyses of intranasal COVID-19 vaccines. Fewer mice immunised i.t. showed these responses, perhaps indicating that intranasal administration is more efficient at generating T cell responses. We found a similar pattern when analysing IFN-γ responding T cells by intracellular cytokine staining, finding both CD4+ and CD8+ populations in the higher i.n. dose group.

Next, we repeated our immunisation of C57BL/6J mice in order to follow up T cell responses over a longer period of time. We continued to see significant CD8+ T cell responses in the higher i.n. group at 6 months post vaccination and observed a similar trend for CD4+ T cell responses. The induction of a strong and durable CD8+ T cell response is often a challenge for subunit vaccines. Also, antibody levels were sustained at high level 6 months post-vaccination.

Overall, our ISR52 vaccine candidate showed good effectivity against a high dose of virus of a different strain than those to which the mice where immunised; this was true for both i.n. and i.t. administration at the higher 20 μg Spike S1 dose. Further it demonstrated a broad immune response both in terms of cross-reactive antibodies and a long-term spike peptide specific T cell response. Based on these promising virus challenge, serological, and cellular immunity data, we are moving forward with a dry powder formulation of ISR52 for nasal administration or inhalation in Phase I/II clinical trials.

### General

It should be understood that any feature and/or aspect discussed above in connections with the compounds or compositions or any other aspect according to the invention apply by analogy to the methods and uses described herein.

The following figures and examples are provided below to illustrate the present invention. They are intended to be illustrative and are not to be construed as limiting in any way.

### BRIEF DESCRIPTION OF THE FIGURES

FIG. 1—A. Primary amino acid sequence of ISR52-DS (SEQ ID NO: 7). The N-Terminal Domain NTD (magenta), Receptor Binding Domain RBD (green) and SD1-SD2 region (grey) is highlighted. B. 3D structural representation of the ISR52-DS highlighting the same color-coded regions as with the primary amino acid sequence. PDB accession number 7A92 was used for preparing this schematic representation.

FIG. 2—Relative body weights following immunisation and inoculation with SARS-CoV-2. Animals were inoculated on Day 28. i.n: intranasal; i.t.: intratracheal; s.c.: subcutaneous.

FIG. 3—Survival of hACE2 mice (untreated controls vs. immunised animals) following SARS-CoV-2 virus challenge. hACE2: Human angiotensin-converting enzyme 2.

FIG. 4—IgG titres against Spike (Wuhan=original strain), RBD (South Africa=beta) and RBD (UK=alpha) following immunisation (geometric mean±geometric SD). Samples were acquired on Day 28. Immunisation was performed on Day 0 and 14. One animal in Group 6 was excluded as it had received only one dose of the candidate vaccine. RBD: Receptor-binding domain.

FIG. 5—IgA titres against Spike (Wuhan=original strain), RBD (South Africa=beta) and RBD (UK=alpha) following immunisation (geometric mean±geometric SD). Immunisation was performed on Day 0 and 14. Samples were acquired on Day 28. One animal in Group 6 was excluded as it had received only one dose of the candidate vaccine. RBD: Receptor-binding domain.

FIG. 6—Total immunoglobulin titres (geometric mean±geometric SD) against RBD following immunisation (Day 0 and Day 14). Samples were collected at termination. One animal in Group 6 was excluded as it had received only one dose of the candidate vaccine. RBD: Receptor-binding domain.

FIG. 7—IgG titres against Spike (Wuhan=original strain), RBD (SA=beta) and RBD (UK=alpha) in BAL (geometric mean±geometric SD). Immunisation was performed on Day 0 and Day 14. Samples were acquired at termination. One animal in Group 6 was excluded as it had received only one dose of the candidate vaccine. BAL: Bronchoalveolar lavage. RBD: Receptor-binding domain.

FIG. 8—IgA titres against Spike (Wuhan=original strain), RBD (SA=beta) and RBD (UK=alpha) in BAL (geometric mean±geometric SD). One animal in Group 6 was excluded as it had received only one dose of the candidate vaccine. BAL: Bronchoalveolar lavage. RBD: Receptor-binding domain.

FIG. 9—Immunoglobulin G (IgG) and immunoglobulin A (IgA) titres against SARS-CoV-2 RBD alpha and beta as well as SARS-CoV-2 Spike S1 (Wuhan=original strain) in serum and bronchoalveolar lavage (BAL) fluid following administration of test item (20 μg trimeric SARS-CoV-2 Spike+10 μg Poly IC or Poly IC:LC+40 μg Vitamin A; Group 1 and Group 2, respectively). Serum IgG titres were determined two weeks after the first (Day 14) and second (Day 28) administration of test item. Serum IgA titres as well as IgG and IgA levels in BAL fluid were determined two weeks after the second administration of test item. Data is presented as arithmetic means. RBD: Receptor-binding domain.

FIG. 10—Immunoglobulin G (IgG) titres against SARS-CoV-2 RBD alpha and beta as well as SARS-CoV-2 Spike S1 (Wuhan=original strain) in serum two weeks after the first (Day 14) and second (Day 28) administration of test item containing different doses of Poly IC:LC. Data is presented as arithmetic means. RBD: Receptor-binding domain.

FIG. 11—Immunoglobulin G (IgG) titres against SARS-CoV-2 RBD alpha and beta as well as SARS-CoV-2 Spike S1 (Wuhan=original strain) in bronchoalveolar lavage (BAL) fluid two weeks after the second administration of test item containing different doses of Poly IC:LC. Data is presented as arithmetic means. RBD: Receptor-binding domain.

FIG. 12—Immunoglobulin A (IgA) titres against SARS-CoV-2 RBD alpha and beta as well as SARS-CoV-2 Spike S1 (Wuhan=original strain) in serum and bronchoalveolar lavage (BAL) fluid two weeks after the second administration of test item containing different doses of Poly IC:LC. Data is presented as arithmetic means. RBD: Receptor-binding domain.

FIG. 13—Immunoglobulin G (IgG) and immunoglobulin A (IgA) titres against SARS-CoV-2 RBD alpha and beta as well as SARS-CoV-2 Spike S1 (Wuhan=original strain) in serum and bronchoalveolar lavage (BAL) fluid following administration of test item (20 μg monomeric SARS-CoV-2 Spike+10 μg Poly IC:LC) with (Group 4) and without (Group 8) 40 μg Vitamin A. Serum IgG titres were determined two weeks after the first (Day 14) and second (Day 28) administration of test item. Serum IgA titres as well as IgG and IgA levels in BAL fluid were determined two weeks after the second administration of test item. Data is presented as arithmetic means. (RBD: Receptor-binding domain).

FIG. 14—Immunoglobulin G (IgG) and immunoglobulin A (IgA) titres against SARS-CoV-2 RBD alpha and beta as well as SARS-CoV-2 Spike S1 (Wuhan=original strain) in serum and bronchoalveolar lavage (BAL) fluid following administration of test item (Group 2: 20 μg trimeric SARS-CoV-2 Spike+10 μg Poly IC:LC+40 μg Vitamin A; Group 3: 20 μg SARS-CoV-2 RBD mix+10 μg Poly IC:LC+40 μg Vitamin A; Group 4: 20 μg monomeric SARS-CoV-2 Spike+10 μg Poly IC:LC+40 μg Vitamin A). Serum IgG titres were determined two weeks after the first (Day 14) and second (Day 28) administration of test item. Serum IgA titres as well as IgG and IgA levels in BAL fluid were determined two weeks after the second administration of test item. Data is presented as arithmetic means. (RBD: Receptor-binding domain).

FIG. 15—Relative body weights following immunisation and inoculation with SARS-CoV-2. Animals were inoculated on Day 28. HD: high-dose; i.n.: intranasal; i.t.: intratracheal; LD: low-dose; p.i.: post-infection.

FIG. 16—Survival of hACE2 mice (untreated controls vs. vaccinated animals) following SARS-CoV-2 virus challenge. Statistical analysis was performed using the Log-Rank (Mantel-Cox) test. HD: high-dose; i.n.: intranasal; i.t.: intratracheal; LD: low-dose.

FIG. 17—IgG titres against Spike (Wuhan=original strain), RBD (SA=beta) and RBD (UK=alpha), S1 RBD2 (Delta) and S1 RBD2 (Kappa) following immunisation (geometric mean±geometric SD). Samples were acquired on Days 14 and 28 or at termination. Immunisation was performed on Day 0 and 14. HD: high-dose; i.n.: intranasal; i.t.: intratracheal; LD: low-dose; RBD: receptor-binding domain.

FIG. 18—IgA titres against Spike (Wuhan=original strain), RBD (SA=beta) and RBD (UK=alpha), S1 RBD2 (Delta) and S1 RBD2 (Kappa) following immunisation (geometric mean±geometric SD). Samples were acquired on Days 14 and 28 or at termination. Immunisation was performed on Day 0 and 14. HD: high-dose; i.n.: intranasal; i.t.: intratracheal; LD: low-dose; RBD: receptor-binding domain.

FIG. 19—IgG titres against Spike (Wuhan=original strain), RBD (SA=beta) and RBD (UK=alpha) in BAL (geometric mean±geometric SD). Immunisation was performed on Day 0 and Day 14. Samples were acquired at termination. HD: high-dose; i.n.: intranasal; i.t.: intratracheal; LD: low-dose; RBD: receptor-binding domain.

FIG. 20—IgA titres against Spike (Wuhan=original strain), RBD (SA=beta) and RBD (UK=alpha) in BAL (geometric mean±geometric SD). Immunisation was performed on Day 0 and Day 14. Samples were acquired at termination. HD: high-dose; i.n.: intranasal; i.t.: intratracheal; LD: low-dose; RBD: receptor-binding domain.

FIG. 21—Spike-specific interleukin 2 (IL-2)- or interferon γ (IFN γ)-secreting splenocytes (mean±SEM), as represented by number of spots, following a single (D.14) or double (D.28) intranasal (i.n.) or intratracheal (i.t.) administration with low dose (5 μg Spike+10 μg Poly IC:LC, LD) or high dose (20 μg Spike+10 μg Poly IC:LC, HD) test item. *p<0.05, **p<0.01, ***p<0.001 as analysed using a Kruskal-Wallis non-parametric ANOVA, followed by a Dunn's multiple comparison post-hoc test.

FIG. 22—Percent spike-specific interferon γ (IFNγ)⁺ T-cells (mean±SEM), following double intranasal (i.n.) or intratracheal (i.t.) administration with low dose (5 μg Spike+10 μg Poly IC:LC, LD) or high dose (20 μg Spike+10 μg Poly IC:LC, HD) test item. **p<0.01 as analysed using a Kruskal-Wallis non-parametric ANOVA, followed by a Dunn's multiple comparison post-hoc test.

FIG. 23—Relative body weights (mean±SEM) following one (Cohort A) or two (Cohort B) intranasal (i.n.) or intratracheal (i.t.) administrations with low dose (5 μg Spike+10 μg Poly IC:LC, LD) or high dose (20 μg Spike+10 μg Poly IC:LC, HD) test item. Administration was performed on Day 0 and 14.

FIG. 24—Circulating immunoglobulin IgG titres (geometric mean±95% CI) following a one (Day 14) or two (D.28) intranasal (i.n.) or intratracheal (i.t.) administrations with low dose (5 μg Spike+10 μg Poly IC:LC, LD) or high dose (20 μg Spike+10 μg Poly IC:LC, HD) test item.

FIG. 25—Immunoglobulin (IgG) titres (geometric mean±95% CI) in bronchoalveolar lavage (BAL) fluid following intranasal (i.n.) or intratracheal (i.t.) administration with low dose (5 μg Spike+10 μg Poly IC:LC, LD) or high dose (20 μg Spike+10 μg Poly IC:LC, HD) test item on two occasions.

FIG. 26—A-J show ELISA results showing cross reactivity with Spike Wuhan, Alpha, Delta and Omicron.

FIG. 27—Immunoglobulin G (IgG) titres against SARS-CoV-2 Spike-RBD2 alpha and SARS-CoV-2 Spike RBD2 delta in serum and broncheoalveolar lavage (BAL) fluid. Animals received two doses of low-dose (5 μg Spike+10 μg Poly IC:LC, LD) or high-dose (20 μg Spike+10 μg Poly IC:LC, HD) test item via intranasal (i.n.) or intratracheal (i.t.) administration.

FIG. 28—Percent Spike-specific IFNγ⁺ T-cells (mean±SEM), following two administrations of intranasal (i.n.) or intratracheal (i.t.) low dose (5 μg Spike+10 μg Poly IC:LC, LD) or high dose (20 μg Spike+10 μg Poly IC:LC, HD) test item. p<0.01 as analysed using a Kruskal-Wallis non-parametric ANOVA, followed by a Dunn's multiple comparison post-hoc test.

FIG. 29—shows robust anti-Spike IgA in BAL from the high dose i.n. group, with weak induction present in the s.c and i.t. groups. Anti-Spike S1 IgA was analysed from BAL fluid harvested at termination. BAL was diluted 1:30 and the optical density (OD) is shown for individual mice.

FIG. 30—shows neutralizing antibodies from BAL fluid at termination. BAL was then diluted beginning at 1-in-4 (LoD) and tested for neutralization of SARS-CoV-2 infection of Vero E6 cells. Results for each individual are plotted.

FIG. 31—shows the presence of SARS-CoV-2 RNA at the time of euthanasia in BAL and lung tissue. Bronchoalveolar lavage fluid (BAL), and organs were harvested from mice at the day of termination. SARS-CoV-2 RNA in BAL was detected from mice which died during infection, including control mice and 4 mice from the low-dose s.c. group. Meanwhile, low to no detectable viral RNA was found in mice that survived (Ct>35). Similar results were obtained with lung tissue, though fewer animals overall had quantifiable SARS-CoV-2 RNA. These results indicate that the vaccinated animals, with the exception of the low dose s.c. group, were cleared from the infection by day 11 post infection. SARS-CoV-2 RNA levels in BAL fluid and lung tissue at termination. 1 mL of BAL fluid for each mouse was collected at the day of euthanasia. Of this fluid, 90 μL was subjected to RNA extraction and SARS-CoV-2 E gene RT-qPCR. Ct values are plotted for each vaccine group on the left y axis. A section of lung tissue was harvested at termination for RNA and 5 ng of RNA was subsequently analysed by RT-qPCR for E gene copy number (shown on right y axis).

FIG. 32—shows that the histopathology analysis revealed inflammation and necrosis in brain tissue for unvaccinated (8/8) and both low (5/7) and high (2/7) dose subcutaneously vaccinated groups; no similar observations were made for animals vaccinated by the respiratory routes. These results demonstrates that both i.n. and i.t. administration of ISR52, even at low dose, protect from signs of neuronal damage in the hACE2 mouse model. Histopathology scoring of perivascualar inflammatory cell infiltration in striatum-level meninges tissue from the brains of the challenged mice at termination

FIG. 33 shows SEQ ID NO: 3, ISR52

FIG. 34 shows SEQ ID NO: 7, ISR52SD

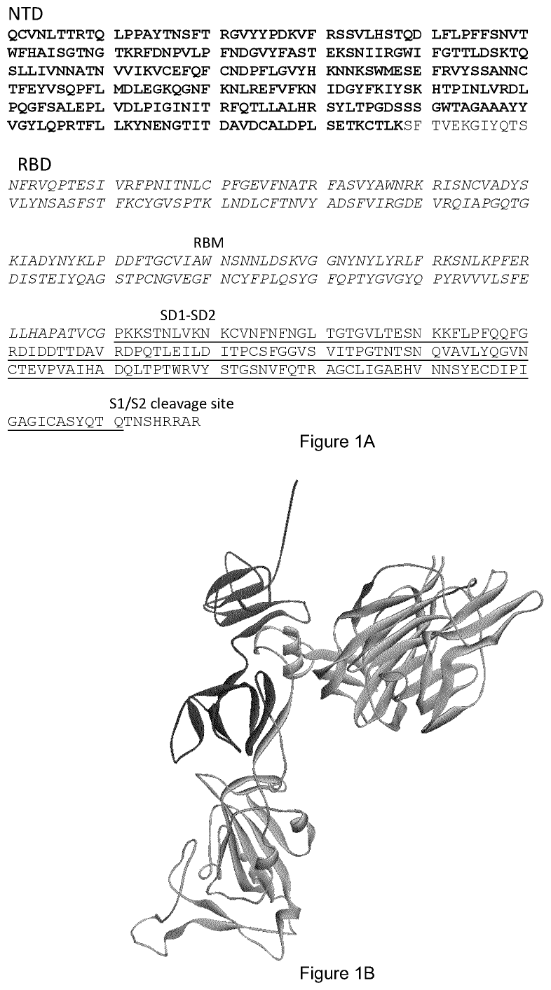

### SPECIFIC EMBODIMENTS

1. A vaccine composition comprising one or more proteins expressed on the surface of a respiratory virus or bacterium and one or more pharmaceutically acceptable excipient, wherein the composition is in particulate form having a mean particle size in a range of from 2 to 50 μm.

2. A vaccine composition according to item 1, wherein the protein is present in its three-dimensional structure.

3. A vaccine composition according to item 1 or 2 comprising one or more pharmaceutically acceptable excipients to ensure flowability, to ensure protein structure, to ensure protein stability, to avoid intra-particle cohesivity, and/or to avoid agglomeration.

4. A vaccine composition according to any one of the preceding items, wherein the respiratory virus is selected from influenza virus, respiratory syncytial virus, parainfluenza virus, metapneumovirus, rhinovirus, coronavirus, adenovirus, and bocavirus.

5. A vaccine composition according to any one of the preceding items, wherein the respiratory virus is a coronavirus.

6. A vaccine composition according to any one of the preceding item, wherein the one or more proteins is a spike protein of a coronavirus such as full-length spike protein of COVID-19 or variants tor fragments hereof.

7. A vaccine composition according to any one of items 1-3, wherein the respiratory bacterium is selected from Streptococcus pneumoniae, Mycoplasma pneumonia, Haemophilus influenza, Chlamydophilia pneumoniae, Chlamydia Psittaci, Moraxella catarrhalis, Mycobacterium tuberculosis, Mycobacterium avis, and Mycobacterium marinum.

8. A vaccine composition according to any one of the preceding items designed for nasal administration.

9. A vaccine composition according to any one of the preceding items, wherein the mean particle size is in a range of from 20 to 50 μm such as from 30 to 40 μm and with a size distribution revealing less than 10% of the particles have a particle size of 10 μm or less.

10. A vaccine composition according to any one of items 1-7 designed for inhalation.

11. A vaccine composition according to any one of items 1-7, 10, wherein the mean particle size is 10 μm or less such as at the most 8 μm, at the most 6 μm, at the most 5 μm or in a range of from 1 μm to 5 μm such as in a range of from 3 μm to 5 μm.

12. A vaccine composition according to any one of the preceding items having a suitable flowability when measured according to the method described in 2.9.16 of Ph.Eur. 10.0 using a funnel without stem and nozzle 1 with a diameter of 10±0.01 mm.

13. A vaccine composition according to any one of the preceding items, wherein the one or more pharmaceutically acceptable excipient is selected from cellulose, cellulose derivatives, methylcellulose, hydroxypropyl cellulose, hydroxypropyl methyl cellulose, microcrystalline cellulose, saccharides including monosaccharides, disaccharides, oligosaccharides, polysaccharides, amino acids including peptides, and mixtures thereof.

14. A vaccine composition according to any one or the preceding items, wherein the one or more pharmaceutically acceptable excipient is selected from disaccharides, oligosaccharides, amino acids, peptides and polypeptides.

15. A vaccine composition according to item 14, wherein the disaccharides are selected from trehalose, sucrose, lactose.

16. A vaccine composition according to item 15, wherein the disaccharide is trehalose.

17. A vaccine composition according to item 14, wherein the oligosaccharide is a cyclodextrin.

18. A vaccine composition according to item 17, wherein the cyclodextrin is a beta-cyclodextrin such as hydroxypropyl-beta-cyclodextrin.

19. A vaccine composition according to item 14, wherein the amino acid is selected from lysine and/or the peptide is selected from tri-lysine and/or the polypeptide is selected from polylysine.

20. A vaccine composition according to any one of the preceding items further comprising an adjuvant.

21. A vaccine composition according to any one of the preceding items further comprising a TLR agonist.

22. A vaccine composition according to item 21, wherein the TLR agonist is a TLR2 agonist and/or TKR3 agonist.

23. A vaccine composition according to item 21 or 22, wherein the TLR agonist is a TLR3 agonist.

24. A vaccine composition according to any one of item 21-23, wherein the TLR agonist is a TLR3 agonist selected from Poly IC and Poly IC:LC.

25. A vaccine composition according to any one of the preceding items further comprising vitamin A and/or vitamin D. or in combination with A and/or D.

### Materials and Methods

### ISR52

The ISR52 vaccine is based on SARS-CoV-2 Spike S1 Alpha protein and the adjuvant Poly IC:LC. During the development of the vaccine, presented herein, different Spike variants have been evaluated. Each Spike protein was manufactured by Icosagen (Tartu, Estonia). For the first challenge study, SARS-CoV-2 trimeric Spike was used (Cat. no P-309-100); for the second challenge study, monomeric SARS-CoV-2 Spike S1 VOC 202012/K (alpha) (Cat. no P-310-100) and monomeric SARS-CoV-2 Spike S1 VOC 501.V2 (beta) (Cat. no P-319-100) were used. In the T-cell response study, monomeric SARS-CoV2 Spike S1 B.1.17 (Similar to Cat. no P-310-100, but without His-tag) was used. For the first challenge, all-trans retinoic acid (Sigma-Aldrich) was used as a supportive substance, 40 μg per mouse, with 10 μg poly IC (Invivogen) as an adjuvant. For the remaining studies, 10 μg poly IC:LC (Hiltonol, Oncovir) was used as an adjuvant.

### Virus

SARS-CoV-2 virus strain SARS-CoV-2/01/human/2020/SWE (Monteil et al., 2020) is described here as the Founder strain. SARS-CoV-2 virus strain hCoV-19/Sweden/21-51217/2021 (lineage B.1.351) (Normark et al., 2021) is described here as the Beta strain. SARS-CoV-2 virus strain SARS-CoV-2/hu/DK/SSI-H13 was provided by Dr Charlotta Polacek Strandh, Statums Serum Institut, Copenhagen, Denmark as described herein as the delta strain. All SARS-CoV-2 strains were grown and titered in a BSL-3 facility on VeroE6 cells by plaque assay (Becker et al., 2008; Monteil et al., 2020) or TCID50 using the presence of cytopathic effect (CPE) as the endpoint (Wulff et al., 2012).

### Animal Experiments

Female AC70 hACE2 mice (Tseng et al., 2007) were purchased from Taconic, Denmark. Treatment of the mice was governed by ethical permit no 16765-2020 approved by the regional animal experimental ethics committee in Stockholm and animals that have reached the humane endpoint according to the granted ethical application will be euthanised and blood, bronchoalveolar lavage and tissues will be sampled from such animals and analysed according to the study plan and all animal experiments were conducted at Astrid Fagraeus Laboratorium under BSL-3 conditions. Isofluorane anaesthesisa was used for intransal vaccination, intransal infection, euthanasia, and broncheoalveolar lavage. Ketaminol and Rompun anaesthesia was used for intratracheal vaccination, and once mice were fully anaesthetised, they were placed in a supine position. A bent 19 G steel gavage tube was used to administer 25 μL of vaccine between the vocal cords. Indicated adjuvants Poly(I:C) (HMW) (Cat. no vac-pic, Invivogen), ATRA (Cat no: R2625, Sigma Aldrich), and/or Poly IC:LC (Hiltonol, Oncovir Inc) were mixed with antigen just prior to immunisation. Mice were infected with the indicated infectious dose of SARS-CoV-2 intranasally in a volume of 25 μL. Health status was documented on vaccination days and daily after infection according to Irwin screen.

### Cells

VeroE6 cells were obtained from ATCC (CRL-1586) and maintained in DMEM media supplemented with 10% fetal calf serum (FCS; Gibco), 1% Non-essential amino acids (Gibco), and 10 mM HEPES (Gibco) at 37° C. in a 5% CO₂ humidified atmosphere.

### Microneutralization Assay

VeroE6 cells were seeded on 96 well plates at a seeding density of 2×10⁴ cells per well one day prior to infection. BAL or serum was heat inactivated for 30 minutes at 56° C. before dilution in DMEM media supplemented with 2% FCS, 100 units/mL of penicillin and 100 μg/mL streptomycin. The diluted BAL/serum was then distributed in triplicate into 96 well round bottom plates and mixed with 500 PFU (plaque forming units) of SARS-CoV-2 per well and incubated at 37° C./5% CO₂ for 1 hour. Media from the VeroE6 cells was aspirated and replaced with virus:BAL/serum mix. Virus only, BAL/serum only, and convalescent patient serum controls were included. After 3 days' incubation at 37° C./5% C02, the presence of CPE was checked. Neutralising activity was assigned to wells which showed a complete absence of CPE. The neutralising titer was then calculated as the dilution factor where >50% of wells showed neutralising activity.

### ELISA

Clear flat bottom immuno nonsterile 96 well polystyrene plates (Thermo Scientific) were coated with either founder strain Spike S1, alpha variant RBD2, beta variant RBD2, delta variant RBD2 or omicron variant RBD2 purchased from Icosagen (Tartu, Estonia) at 5 μg/ml in PBS 1× buffer, pH 7.2, using 50 μl/well. The plates were covered with sealing tape and incubated o/n at 4° C. on clear flat bottom immuno nonsterile 96 well polystyrene plates (Thermoscientific) at 5 μg/ml (50 μl/well) in PBS 1× (pH 7.2) buffer. Then plates were washed with PBS+0.05% Tween 20 and incubated with 150 μl/well of a blocking solution (Diluent, Mabtech) postcoat for 1 h at RT. Plates are washed and then 100 μl of desired diluted serum in diluent (Mabtech) were incubated for 2 h at RT, followed by washing. Detection antibody, 100 μl/well, diluted 1:1000 in diluent was added and incubated for 1 h at RT. For IgG, conjugated anti-mouse IgG alkaline phosphatase (ALP) (Cat. no 3310-4, Mabtech) was used, and for IgA first biotinylated anti-mouse IgA (100 μl/well, diluted 1:1000 in diluent) (Cat. no 3865-6, Mabtech) and then streptavidin ALP (100 μl/well, diluted 1:1000 in diluent) (Cat. no 3310-8, Mabtech) was added and incubated for 1 h at RT. After washes, 100 μl pNPP substrate (Mabtech) per well was added, the enzymatic reaction was developed for 30 minutes, and analyte absorbance (410 nm) and background absorbance (620 nm) measured by BMG LABTECH FLUOstar omega ELISA plate reader. Results were considered positive when the optical density (OD) obtained with ELISA the mAbs was three times greater than the negative control. All antibodies, the diluent solution and the pNPP substrate were from Mabtech.

### IL-2 and IFN-γ ELISpot

IL-2 and IFN-γ ELISpot reagents were purchased from Mabtech and the assays were performed according to manufacturer instructions. Mabtech's mouse IL-2 ELISpot PLUS kit (ALP) and mouse IFN-γ ELISpot PLUS kit (ALP) were used for detection of IL2 and IFN-γ, respectively. The precoated plates are washed 4 times with sterile PBS pH 7.2 (Gibco Life Technologies Limited) and conditioned by blocking with 10/90% FBS/RPMI 1640 Glutamax (Gibco Life Technologies Limited) for at least 30 min at room temperature. SARS CoV2 S1 scanning pool antigen stimuli stock solution (Mabtech), 200 μg/ml, was prepared from the lyophilized peptide pool by addition of 40 μL of DMSO and 85 μL PBS followed by further dilution in cell culture medium giving a peptide concentration of 2 μg/ml. Conditioning medium was removed from the plates and 100 μL of peptide pool, followed by 100 μL of cell suspension, such that 250,000 mouse splenocytes were stimulated per well. The plates were incubated at 37° C., 5% CO₂ for 15-18 hours for IL2 plates and 32-35 hours for IFNγ plates. The next day cells were removed and the plates washed five times followed by incubation with 100 μl/well of detection antibody (5H4-biotin) diluted to 1 μg/ml in PBS containing 0.5% FBS, for IL-2 detection and detection antibody R4-6A2-biotin for IFN-γ detection, for 2 hours at room temperature. The plates were then washed five times with PBS followed by incubation with 100 μl/well of Streptavidin-ALP diluted 1:1000 in PBS containing 0.5% FBS. After incubation at room temperature for 1 hour, the plates were washed as before and 100 μL/well of the substrate (BCIP/NBT-plus) was added and the plates incubated for 15 minutes at room temperature in the dark. After extensive washing in tap water, the plates were dried overnight before detection of spot forming units using either an IRIS or ASTOR ELISpot instrument.

### In Vitro Proliferation and Intracellular Cytokine Staining (ICS) Analysis by Flow Cytometry

Splenocytes from vaccinated and control mice were prepared and stained with CellTrace Violet dye according to the manufacturer's instructions (ThermoFisher Scientific, MA, USA). One million splenocytes were then stimulated or left untreated for 4 days in wells of a flat bottom 96 well plate in duplicate. After 4 days, the cells were restimulated with antigen for a further 24 hours. At 6-8h prior to harvesting, protein transport inhibitor cocktail was added to the cells (ThermoFisher Scientific). The cells were harvested, and the duplicate wells pooled into V bottom 96 well plates to reduce cell loss during the intracellular staining. The cell samples were then stained with T-cell surface markers (CD3 FITC, CD8 PerCp-Cy5.5, CD4 APC, all ThermoFisher Scientific) followed by intracellular staining of IFN-gamma (IFNg PE clone XMG1.2. ThermoFisher Scientific) using the intracellular fix/perm kit (ThermoFisher Scientific) according to the manufacturer's instructions. The samples was analyzed with a MACSQuant16 instrument (Miltenyi Biotech).

### SARS-CoV-2 RT-qPCR

Equal volumes of BAL from each mouse were mixed with Trizol (Thermo Fisher). RNA was extracted using Direct Zol RNA mini kit (Zymo research) and analyzed for the content of SARS-CoV-2 RNA by RT-qPCR using the following primers/probes as previously described (Monteil et al., 2020). SARS-CoV-2 E gene:

forward:
ACAGGTACGTTAATAGTTAATAGCGT;
reverse:
ATATTGCAGCAGTACGCACACA;
probe:
FAM-ACACTAGCCATCCTTACTGCGCTTCG-MGB

Lung tissue was lysed in Trizol and RNA was extracted using Direct Zol RNA mini kit (Zymo research). The copy number of SARS-CoV-2 RNA was determined by analysing 5 ng of RNA from each lung tissue sample, using E gene RNA transcripts of a defined copy number (EDX SARS-CoV-2 Standard, Biorad) to generate a standard curve. Primers and probes as above.

### Histopathology

Lung and brain tissue was harvested and fixed in 4% formaldehyde and sectioned (between 4-6 μm). Sections were stained with haematoxylin-eosin and scored for histopatholigic changes according to distribution, severity (grades 1 to 5, minimal, slight, moderate, marked, and severe, respectively, or P for present, and morphologic character.

### Summary and Conclusion of the Tests Made

Based on available immunogenicity data together with the cross-reactivity of IgG and IgA antibodies observed in the non-clinical studies, it is expected that immunisation with SARS-CoV-2 Spike S1 alpha as the only antigen has comparable efficacy as immunisation with other Spike variants. The full-length Spike S1 protein used as antigen in the ISR52 vaccine enables broad immune responses towards different Spike variant. Covid-19 epidemiology is continuously changing with the emergence of new SARS-CoV-2 variants. These variants may carry mutations in antigenic regions which constitutes a challenge for vaccine developers. The SARS-CoV-2 alpha variant carries a mutation in the RBD of the Spike protein which increases binding affinity to the ACE2 receptor which may at least partially explain the higher infectivity of this strain compared to the original Wuhan strain (Zahradnik et al., 2021). The Spike alpha protein shares this N501Y mutation in the RBD with the beta, gamma and omicron (B.1.1.529) variants as well as the D₆₁₄G mutation with the beta, gamma, delta and omicron (B.1.1.529) variants.

### SEQUENCE LISTING

| | |
|---|---|
| SEQ ID NO: 1 | Full length spike protein |
| | Spike_SARS2 |
| | https://www.uniprot.org/uniprot/P0DTC2 |
| SEQ ID NO: 2 | SARS-CoV-2 Spike S1 |
| SEQ ID NO: 3 | ISR52 |
| SEQ ID NO: 4 | Forward primer |
| SEQ ID NO: 5 | Reverse primer |
| SEQ ID NO: 6 | Probe |
| SEQ ID NO: 7 | ISR52-DS |

### EXAMPLES

### Example 1—Vaccine Composition

TABLE
Composition of SARS-COV-2 spike protein
powder for nasal administration, 30 μg

| | Quantity | | Reference |
|---|---|---|---|
| | per Device | | to |
| Component | (mg) | Function | Standard |
| SARS-CoV-2 Spike Protein | 0.030 | Active | |
| | | Pharmaceutical | |
| | | Ingredient | |
| Poly IC:LC | 0.015 | Adjuvant | |
| Trehalose | 4.912 | Filler, | Ph. Eur. |
| | | stabiliser | |
| Hydroxypropyl-beta- | 4.912 | Filler | Ph. Eur. |
| cyclodextrin (Kleptose HPB) | | | |
| Components from input Poly | 0.116 | — | |
| IC:LC suspension (Poly-L- | | | |
| Lysine, Carboxymethyl | | | |
| cellulose, sodium chloride) | | | |
| Components from input | 0.015 | — | |
| Spike Protein solution | | | |
| (sodium chloride, potassium | | | |
| chloride, disodium | | | |
| phosphate, monopotassium | | | |
| phosphate) | | | |
| Total per device | 10.00 | | |

### Batch Formula

The drug product, SARS-CoV-2 spike protein powder for nasal administration, is manufactured in 2 stages.

Initially, the drug substance is used to produce a SARS-CoV-2 Spike Protein feed solution, which is then spray dried to produce a bulk drug product. This bulk drug product is then filled into devices to produce the finished drug product.

The batch formula for a typical batch of SARS-CoV-2 Spike Protein feed solution is shown below.

### Batch Formula for SARS-CoV-2 Spike Protein Feed Solution

| | Quantity per batch |
|---|---|
| Component | (mL/g) |
| SARS-CoV-2 Spike Protein (20 mg/mL) | 0.750 mL (equivalent to |
| | 15 mg Spike Protein) |
| Poly IC:LC suspension (1.8 mg/mL) | 4.167 mL (equivalent to |
| | 7.5 mg Poly IC:LC) |
| Trehalose Dihydrate | 2.715 g (equivalent to |
| | 2.456 g trehalose) |
| Hydroxypropyl-beta-cyclodextrin | 2.456 g |
| (Kleptose HPB) | |
| Components from input Poly IC:LC | Added within Poly |
| suspension (Poly-L-Lysine, Carboxymethyl | IC:LC suspension |
| cellulose, sodium chloride) | |
| Components from input Spike Protein | Added within Spike |
| solution (sodium chloride, potassium | Protein solution |
| chloride, disodium phosphate, | |
| monopotassium phosphate) | |
| Water for Injection | 20.000 mL |
| Total per batch (in feed solution) | 5.000 g/~24.917 mL |

### Batch Formula for SARS-CoV-2 Spike Protein Bulk Drug Product

| | Quantity per | |
|---|---|---|
| Component | batch (g) | |
| SARS-CoV-2 Spike Protein | 0.0150 | g |
| Poly IC:LC suspension | 0.0075 | g |
| Trehalose Dihydrate | 2.4560 | g |
| Hydroxypropyl-beta-cyclodextrin (Kleptose HPB) | 2.4560 | g |
| Components from input Poly IC:LC suspension (Poly- | 0.0581 | g |
| L-Lysine, Carboxymethyl cellulose, sodium chloride) | | |
| Components from input Spike Protein solution | 0.0074 | g |
| (sodium chloride, potassium chloride, disodium | | |
| phosphate, monopotassium phosphate) | | |
| Total per batch | 1. | g |

### Description of Manufacturing Process and Process Controls

The manufacturing process consists of the production of a SARS-CoV-2 Spike Protein Feed Solution, which is then dried by a spray drying process to produce a final SARS-CoV-2 Spike Protein Bulk Drug Product. The Bulk Drug Product is then filled and sealed in devices to produce the Final Drug Product.

The required amounts of trehalose dihydrate and hydroxypropyl beta-cyclodextrin are dissolved in the required amount of water for injection (Solution A). The required amount of Poly IC:IC is dispensed into a bulk container (from 1.2 mL vials) and sonicated to mix. The Poly IC:LC bulk suspension is added to Solution A with gentle stirring until mixed, and then the required amount SARS-CoV-2 Spike Protein is added to form the SARS-CoV-2 Spike Protein Feed Solution. The SARS-CoV-2 Spike Protein Feed Solution is then spray dried to produce the SARS-CoV-2 Spike Protein Bulk Drug Product. IPC testing (IPC 1-IPC 2) is performed on the Bulk Drug Product.

The Bulk Drug Product is then filled into devices using an automated precision balance (fill-to-weight). The devices are sealed with foil using a heat sealer, closed and labelled. IPC testing (IPC 3-IPC 4) is performed during the filling and sealing process. Acceptable devices are packaged into appropriate aluminum foil pouches containing a desiccant. Batch release testing is performed a representative sample of SARS-CoV 2 Spike Protein Powder for Nasal Administration.

Manufacturing Process for SARS-CoV 2 Spike Protein Powder for Nasal Administration and Controls

### Controls of Critical Steps and Intermediates

In accordance with EMA/CHMP/QWP/545525/2017 no data is provided.

Process Validation and/or Evaluation

In accordance with EMA/CHMP/QWP/545525/2017 no data is provided.

### Control of Excipients

The pharmacopoeial excipients are indicated in the following table.

### Pharmacopoeial EXCIPIENTS

| Excipient | Pharmacopoeial Standard |
|---|---|
| Trehalose Dihydrate | Ph. Eur. |
| Hydroxypropyl-beta-cyclodextrin | Ph. Eur. |

### Non-Pharmacopoeial Excipients

There is one non-pharmacopoeial excipient included in this formulation:
Poly IC:LC

### Excipients of Animal or Human Origin

No excipients of animal or human origin are included in the IMP formulation.

### Novel Excipients

There are no novel excipients used.

### Example 2—Overview of Non-Clinical Studies

TABLE 1
overview of non-clinical studies

| Study | Animal | | | | | |
|---|---|---|---|---|---|---|
| Objectives | species/ | Antigen¹ | Adjuvant | Route of | | |
| ID | strain | (dose) | (dose) | administration | Status | Main findings |
| AB21-Efficacy | AC70 | 10, 80 or | 10 | i.n., i.t., s.c. | Completed | Immunisation induced |
| 04 | hACE2 | 100 μg | μg Poly I:C | | | local and systemic IgG |
| | transgenic | SARS- | | | | and IgA production as |
| | mice | CoV-2 | | | | well as dose- |
| | | trimeric | | | | dependent local |
| | | Spike S1 | | | | production of |
| | | (original | | | | neutralising antibodies |
| | | strain) | | | | Immunisation did not |
| | | | | | | affect body |
| | | | | | | weight/health status |
| | | | | | | Immunisation (i.n., i.t.) |
| | | | | | | was associated with |
| | | | | | | lack of viral replication |
| | | | | | | and fully protected |
| | | | | | | against Covid-19 |
| | | | | | | disease |
| | | | | | | after i.n. challenge with |
| | | | | | | 1.875 × 10⁵ TCID50 |
| | | | | | | SARS-CoV-2 |
| AB21-Efficacy | AC70 | 5 or 20 μg | 10 μg Poly | i.n., i.t. | Completed | Immunisation induced |
| 31 | hACE2 | monomeric | IC:LC | | (Histopathology | progressive and dose- |
| | mice | SARS-Cov-2 | | | results | dependent IgG and IgA |
| | | Spike S1 | | | pending) | production as well |
| | | (alpha and | | | | as dose- |
| | | beta) | | | | dependent local and |
| | | | | | | systemic neutralising |
| | | | | | | antibody titres |
| | | | | | | Immunisation did not |
| | | | | | | affect body |
| | | | | | | weight/health status |
| | | | | | | Lack of viral |
| | | | | | | replication and partial |
| | | | | | | protection against |
| | | | | | | severe infection |
| | | | | | | after i.n. challenge |
| | | | | | | with 1.05 × 10⁶ TCID50 |
| | | | | | | SARS-CoV-2 |
| AB21-Immunogenicity | C57/BLJ6 | 20 μg | 3, 10 or | i.n. | Completed | Adjuvant Poly IC or |
| 33 | mice | monomeric | 40 μg Poly | | | Poly IC:LC resulted in |
| | | Spike S1, | IC:LC or 10 | | | comparable antibody |
| | | trimeric Spike | μg Poly I:C | | | response. |
| | | S1 or | | | | Vitamin A did not |
| | | RBD mix | | | | further enhance the |
| | | | | | | immune response. |
| | | | | | | Immunisation with |
| | | | | | | monomeric SARS- |
| | | | | | | CoV-2 Spike elicited a |
| | | | | | | comparable immune |
| | | | | | | response as |
| | | | | | | immunisation with |
| | | | | | | trimeric SARS-CoV-2 |
| | | | | | | Spike. |
| AB21-Immunogenicity | C57/BLJ6 | 5 or 20 μg | 10 μg Poly | i.n., i.t. | Completed | Immunisation induced |
| 73 | mice | monomeric | IC:LC | | | CD4⁺ and CD8⁺ Spike- |
| | | SARS- | | | | specific T-cell |
| | | CoV-2 | | | | responses |
| | | Spike S1 | | | | I.n. and i.t. |
| | | alpha | | | | administration induced |
| | | | | | | circulating IgG |
| | | | | | | Local IgG production, |
| | | | | | | particularly following |
| | | | | | | i.n. administration of |
| | | | | | | 20 μg Spike |
| AB21-Immunogenicity | C57/BLJ6 | 5 or 20 μg | 10 μg Poly | i.n., i.t. | Ongoing | |
| 74 (long-term) | mice | monomeric | IC:LC | | | |
| | | SARS- | | | | |
| | | CoV-2 | | | | |
| | | Spike S1 | | | | |
| | | alpha | | | | |

hACE2: Human angiotensin-converting enzyme 2. IgA: immunoglobulin A; IgG: immunoglobulin G; i.n.: intranasal; i.t.: intratracheal; RBD: receptor-binding domain; s.c.: subcutaneous. TCID50: Median tissue culture infectious dose.
¹The Spike protein used in the initial non-clinical studies (AB21-04, AB21-31, AB21-33) contained two additional amino acids to enhance protein secretion and a polyhistidine tag (His-tag) to facilitate purification. Subsequent studies (i.e., AB21-64 onwards) used the natural form of the protein.

### Example 3—AB21-04—A Study to Assess the Efficacy of a COVID-19 Vaccine Against SARS-CoV-2 Infection in hACE2 Transgenic Mice

Study AB21-04 assessed the efficacy of a candidate Covid-19 vaccine against SARS-CoV-2 infection in transgenic mice expressing human ACE2 which renders them susceptible to infection with the virus. The test item used in this study was based on SARS-CoV-2 trimeric Spike protein 51 (original strain), the adjuvant Poly IC VacciGrade (10 μg) and the support substance vitamin A (0.4 mg/mL). For each route of administration (s.c., i.n. or it.), a LD (10 μg) and a HD (80 μg for i.n. and i.t., 100 μg for s.c. administration) of the Spike protein was evaluated. Fifty-five female A70 hACE2 transgenic mice were included in the study and divided into seven groups. Seven animals (Group 2) did not receive any vaccine and served as controls. The remaining 48 animals (Groups 1 and 3-7) received varying doses of the antigen together with the adjuvant and vitamin A on Day 0 and Day 14 or 15 (s.c, i.n. or i.t.) (Table 2).

TABLE 1
Treatment groups in study AB21-04

| | | Dosing | Route of | Number of |
|---|---|---|---|---|
| Group | Treatment | volume (μl) | administration | animals (N) |
| 1 | 10 μg Spike protein (LD) + | 100 | s.c. | 7 |
| | 10 μg Poly I:C + 0.4 mg/mL vitamin | | | |
| | A | | | |
| 2 | Untreated control | — | — | 8 |
| 3 | 100 μg Spike protein (HD) + | 100 | s.c. | 8 |
| | 10 μg Poly I:C + 0.4 mg/mL vitamin | | | |
| | A | | | |
| 4 | 10 μg Spike protein (LD) + | 25 | i.n. | 8 |
| | 10 μg Poly I:C + 0.4 mg/mL vitamin | | | |
| | A | | | |
| 5 | 80 μg Spike protein (HD) + | 25 | i.n. | 8 |
| | 10 μg Poly I:C + 0.4 mg/mL vitamin | | | |
| | A | | | |
| 6 | 10 μg Spike protein (LD) + | 25 | i.t. | 8 |
| | 10 μg Poly I:C + 0.4 mg/mL vitamin | | | |
| | A | | | |
| 7 | 80 μg Spike protein (HD) + | 25 | i.t. | 8 |
| | 10 μg Poly I:C + 0.4 mg/mL vitamin | | | |
| | A | | | |

HD: High-dose. i.n.: intranasal. i.t.: intratracheal. LD: Low-dose. s.c.: subcutaneous.

Two weeks following the second dose (i.e. at Day 28), animals were inoculated with 1.875×10⁵ TCID50 SARS-CoV-2 via i.n. administration. Following infection with SARS-CoV-2, animals were weighed and monitored for changes in health status daily until termination (Day 38 or 39). The Irwin Screen was modified to specifically include body posture (flat, limb extension, hunch-back, rigid, asymmetric), autonomic signs (miosis, mydriasis, ptosis, perspiration, piloerection, salivation), central signs (tremor, head shake, wet-dog shake, aggression) and gross behaviour (motor activity and stereotypy grade −2 to +2) were assessed. Health status and body weight records were summarised descriptively and analysed i) to determine whether treatment caused any adverse effects, ii) to assess the effect of SARS-CoV-2 infection and iii) to determine whether treatment altered the response to SARS-CoV-2.

Blood samples for serum isolation were acquired before vaccination, on Day 14, Day 28 and at termination. At termination, BAL was performed using 1 mL sterile PBS for determination of viral RNA. Spleen, lungs and trachea were excised, and a section of lung (lower airway) and trachea (upper airway) were saved for analysis of viral titres. Lung and skull (for brain and nasopharyngeal tissues) were subjected to histopathological analysis.

One animal in Group 3 received an imperfect first dose of the test item; one animal in Group 6 did not receive the first immunisation, due to lack of test item. Three animals in Group 7 died following the first immunisation (one animal due to an overdose of anaesthesia and the remaining two animals were euthanised due to complications caused by the administration technique). Additionally, one animal in Group 3 was found dead following the second immunisation, due to lack of oxygen caused by failure to properly insert the cage into the rack.

### Body Weight and Health Status

Test item administration did not overly affect animal body weights. A small decline in body weight was evident for animals in Group 7 between Days 0 and 14; however, all groups showed a general increase in body weight following the second vaccination (FIG. 2).

Challenge of non-vaccinated animals with SARS-CoV-2 resulted in a significant decline in body weight. Relative body weights for animals in all vaccinated groups were significantly different as compared to non-vaccinated animals, particularly on Day 32. Non-vaccinated animals had dropped to 85.7±0.7% of their original body weights by Day 32, whereas vaccinated animals remained between 97.0% and 101.7% of their original body weights. One animal in Group 6 (LD, i.t.) demonstrated a similar decline in body weight as non-vaccinated animals, reaching 83.1% of its original body weight by Day 32. This was the animal that had only been immunised on one occasion (Day 14).

Differences in body weight were additionally evident between vaccinated groups. Relative body weights for Groups 1 (LD, s.c.) and 4 (LD, i.n.) were significantly lower than those of Group 5 (HD, i.n.), 6 (LD, i.t.) and 7 (HD, i.t.). One animal in Group 3 (HD, s.c.) also showed a drastic decline and dropped to 84.7% of its initial body weight; however, this animal received an imperfect first dose of the test item.

Vaccine administration did not overtly affect animal health status and no effect on respiratory function was observed upon Test item administration did not overtly affect animal health status and no effect on respiratory function was observed upon visual inspection of the animals. Inoculation with SARS-CoV-2 however was associated with a severe deterioration in health status from four days after inoculation, particularly in the non-vaccinated group (Group 2). Animals presented with hunched posture, piloerection and decreased movements. Two animals showed signs of aggression and two animals had abnormal motor behavior. Due to the drastic deterioration in health status, as well as body weight loss, animals in the non-vaccinated control group (Group 2) were euthanised on Day 32. Vaccinated animals showed few changes in overt health status. Three animals in Group 1 (LD, s.c.) were euthanised on Days 32 or 34, due to presentation of hunched posture, piloerection, increased movement, rigidity, and tremor. Two of the animals showed increased movement indicative of SARS-CoV-2 infection of the central nervous system (CNS). Moreover, one animal in Group 6 (LD, i.t.) presented with symptoms on Day 32. These three animals were consequently euthanised. No overt symptoms were evident for the remaining vaccinated animals.

### Survival Following Virus Challenge

Test item administration statistically significantly improved survival following SARS-CoV-2 challenge (p<0.0001) (FIG. 3). Median survival for non-vaccinated animals was 4 days, which was significantly lower than survival of animals in Group 1 (LD, s.c.; p=0.0002, median survival: 6 days), Group 3 (HD, s.c.; p=0.0002, median survival: undefined), Group 4 (LD, i.n.; p=0.0001, median survival: undefined), Group 5 (HD, i.n.; p=0.0001, median survival: undefined), Group 6 (LD, i.t.; p=0.0002, median survival: undefined) and Group 7 (HD, i.t.; p=0.0005, median survival: undefined).

### Antibody Responses (Serum)

Circulating IgG titres against Spike (from the original strain) and its RBD (alpha and beta) were detected in all immunised groups on Day 28; increases were dose-dependent, with animals receiving HD Spike showing stronger immunological responses (FIG. 4). In comparison, anti-Spike and anti-RBD IgA titres were only detected in groups that were vaccinated via the i.n. or i.t. route. In particular, i.n. administration was associated with significantly increased IgA titres. As opposed to IgG, no dose-dependency was evident (FIG. 5).

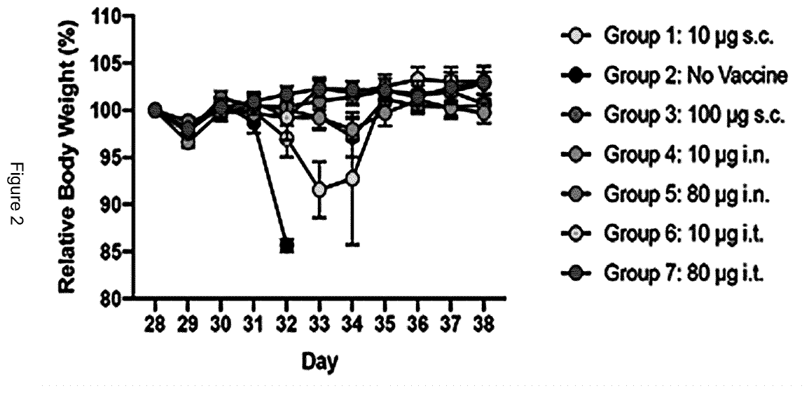

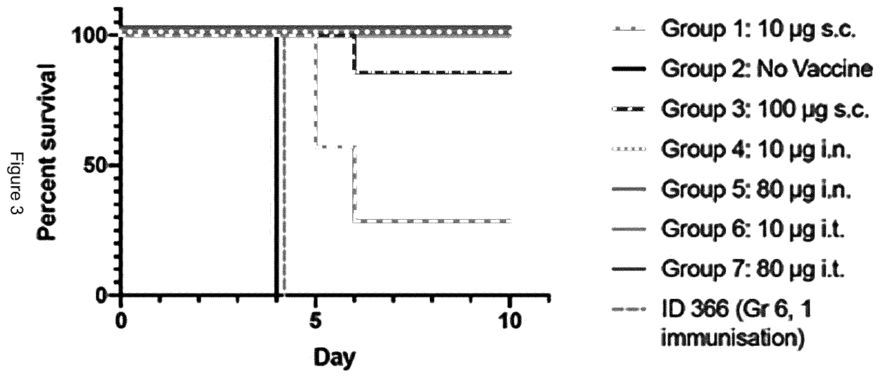

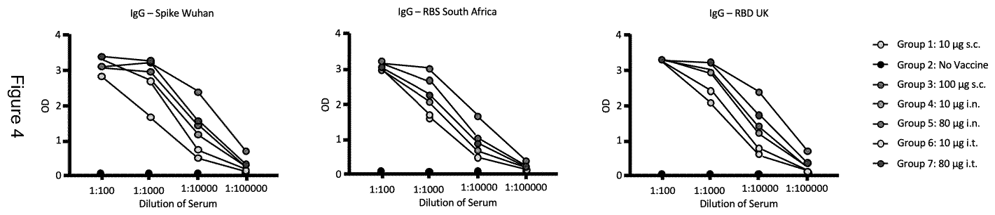

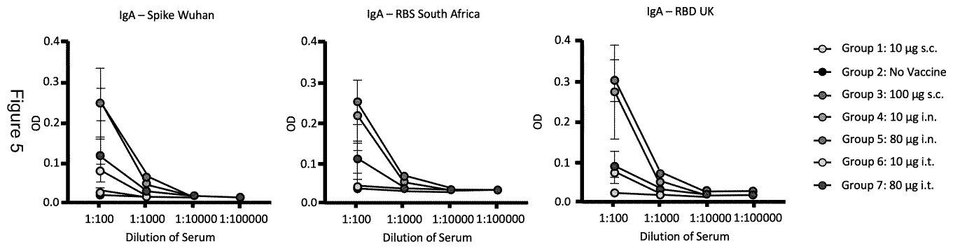

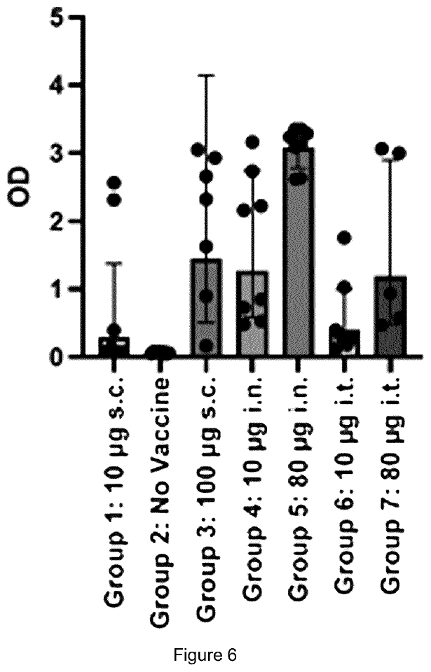

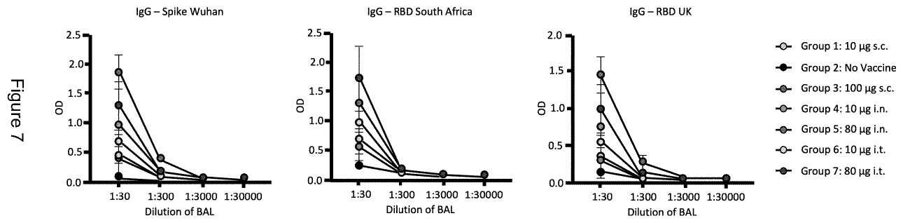

Total immunoglobulin titres against RBD in serum at termination were assessed using a bridge ELISA. Antibodies were detected in all vaccinated groups with the highest titres following i.n. administration. The immunological response was dose-dependent (FIG. 6).

### Antibody Responses (BAL)

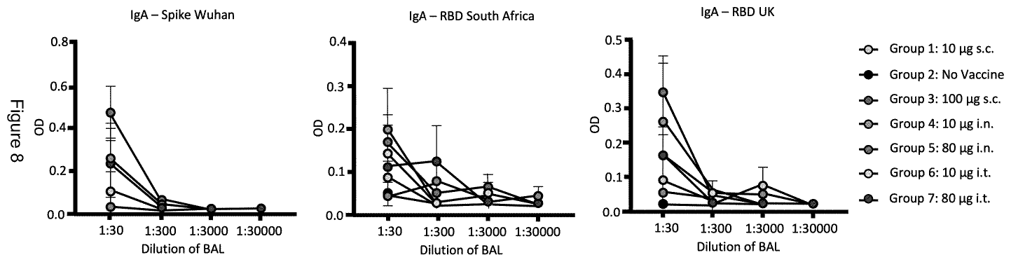

IgG titres against Spike (from the original strain) and RBD (alpha and beta) were detected to some degree in BAL of all vaccinated groups. However, the highest titres were evident in groups that had been vaccinated via the i.n. or i.t. routes, particularly in the HD groups (80 μg Spike). The strongest IgG responses were against Spike and RBD (alpha) (FIG. 7). IgA titres, particularly against Spike (from the original strain) and RBD (alpha) were detected in animals vaccinated via the i.n. route and to a lesser degree in those vaccinated via the i.t. route. A dose-dependent response was evident with animals that had received HD spike showing a stronger immunological response with higher dose (FIG. 8).

### Neutralising Antibodies

Neutralising antibody titres were observed at varying levels in the BAL of vaccinated animals but not in control animals. Among the immunised animals, s.c. administration was associated with the lowest level of neutralising antibodies, with only three animals showing low or partial titres. Intranasal administration was associated with higher neutralising antibody titres. The response was dose-dependent, with three animals in the LD group presenting with low titres (1:4 to 1:16) and six animals in the HD group presenting with medium to high titres (1:4 to 1:32). Intratracheal administration was also associated with detectable neutralising antibodies in the BAL fluid. The effect was, however, not as prominent as that seen following i.n. administration. Two animals in the LD group showed low titres (1:4) and five animals in the HD group showed low to moderate titres (1:4 to 1:8).

### Viral Titres

Viral titres were measured with the quantitative polymerase chain reaction (qPCR) method. SARS-CoV-2 virus (1-2000 copies/mL) was detectable in BAL fluid of all non-immunised control animals, indicative of a successful infection. Low viral titres (C[t] values between 32-35) were detected in three animals that had received s.c. vaccination; these animals also showed low antibody responses. Viral titres were undetectable in animals that were vaccinated via i.n. or i.t. administration suggesting that they were fully protected against SARS-CoV-2 infection.

### Histopathology

No macroscopic lesions related to pathological changes were observed. However, morphological alterations were detected in the nasopharynx, lower respiratory tract and brain.

All groups, including non-immunised controls, showed inflammatory changes in the respiratory tract such as intraluminal fluid and debris in the nasopharynx, possibly due to virus administration. However, in contrast to the other groups, inflammatory cell infiltration in the lower respiratory tract was not detected or detected to a lesser extent in non-immunised animals. Moreover, perivascular to parabronchial and alveolar to interstitial inflammatory cell infiltration was observed to a higher degree in Group 1 (LD, s.c.), and to a slightly higher degree in Groups 5 (HD, i.n.) and Group 7 (HD, i.t.). Only minimal changes were observed in non-immunised controls. In comparison, inflammatory cell infiltration and decreased lumen in arterioles, as well as bronchiolar debris were observed to the highest degree in animals that had been immunised via s.c. administration. These changes were observed to a lesser degree in animals that received i.n. or i.t. immunisation and were not observed in non-immunised controls. As inflammation was minimal in non-immunised controls, inflammatory changes may be evidence of a vaccine-driven anti-viral immune response. Inflammatory changes were also observed in the CNS, namely the striatum; this may explain the abnormal motor behaviours observed in some animals. Neuronal necrosis in the piriform cortex, as well as perivascular inflammatory cell infiltration was observed in the meninges and parenchyma in animals that had been immunised via s.c. administration, as well as in non-immunised animals. These changes were not observed in Groups 4-7, suggesting that i.t. and i.n. administration of test item prevented viral CNS infiltration.

### Summary and Conclusion

In summary, i.n. inoculation with SARS-CoV-2, at 1.875×10⁵ TCID50 resulted in decreased body weight and deteriorated health status of animals which warranted pre-term euthanasia within four days of infection. This was associated with increased viral titres in the lower respiratory tract.

Intranasal and i.t. administration of trimeric Spike (10-80 μg), Poly IC (10 μg) and vitamin A (40 μg) had no overall effect on health status.

Vaccinated animals showed a dose-dependent serological response with systemic and local production of anti-Spike and anti-RBD IgG and IgA antibodies as well as local production of neutralising antibodies. This was associated with a lack of viral replication in the lungs, inhibition of SARS-CoV-2-driven encephalitis and prevention of Covid-19 disease progression.

In conclusion, the study showed that i.n. and i.t. administration of SARS-CoV-2 trimeric Spike protein (10-80 μg), Poly IC (10 μg) and vitamin A on two occasions, fully protected against SARS CoV 2 infection at 1.875×10⁵ TCID50.

### Example 4—AB21-33 a Study to Determine the Immunogenicity of a Novel Covid-19 Vaccine in Mice

This study aimed to assess the immunogenicity of different variants of the antigen with Poly IC or Poly IC:LC as an adjuvant in C57BL/6J wildtype mice. Moreover, the results from this study were used to optimise conditions for subsequent studies and the formulation of the clinical candidate vaccine. The study was approved by the regional animal experimental ethics committee in Stockholm (North), Sweden.

Forty-eight mice were divided into eight treatment groups which received combinations of different antigens (20 μg monomeric or trimeric Spike protein (original strain from Wuhan, China), RBD mix [mix of four antigens including the original strain, alpha, beta and gamma variant]) and adjuvants (10 μg Poly IC or 3-40 μg Poly IC:LC) with or without 40 μg vitamin A (Table 3). Animals were immunised on Day 0 and Day 14 by i.n. administration.

Blood samples were collected on Day 0 and Day 14 (before vaccination) for serum preparation. On Day 28 (i.e., two weeks following administration of the second dose), BAL was performed, and a terminal blood sample was collected before the animals were euthanised. The spleen was collected for T-cell analysis. Animals were monitored for health changes and their body weight was measured on vaccination dates as well as at termination. Animals that exhibited severe health deterioration were terminated prematurely.

TABLE 2
Treatment schedule in Study AB21-33.

| | | Dose | Number of |
|---|---|---|---|
| | | volume | animals |
| Group | Treatment | (μl) | (n) |
| 1 | 20 μg Trimer Spike + | 25 | 6 |
| | 10 μg Poly IC + 40 μg vitamin A | | |
| 2 | 20 μg Trimer Spike + | 25 | 6 |
| | 10 μg Poly IC:LC + 40 μg vitamin A | | |
| 3 | 20 μg RBD-Mix + | 25 | 6 |
| | 10 μg Poly IC:LC + 40 μg vitamin A | | |
| 4 | 20 μg Monomer Spike + | 25 | 6 |
| | 10 μg Poly IC:LC + 40 μg vitamin A | | |
| 5 | 20 μg Monomer Spike + | 25 | 6 |
| | 40 μg Poly IC:LC + 40 μg vitamin A | | |
| 6 | 20 μg Monomer Spike + | 25 | 6 |
| | 3 μg Poly IC:LC + 40 μg vitamin A | | |
| 7 | 20 μg Monomer Spike + | 25 | 6 |
| | 40 μg vitamin A | | |
| 8 | 20 μg Monomer Spike + | 25 | 6 |
| | 10 μg Poly IC:LC | | |

RBD: Receptor-binding domain.

### Comparison of Poly IC and Poly IC:LC

The Applicant's first non-clinical study AB21-04 used Poly IC as an adjuvant. However, Poly IC is not approved for clinical use in humans. Therefore, study AB21-33 evaluated whether Poly IC:LC, a preparation of Poly IC for clinical use, could substitute the variant of Poly IC in the candidate vaccine formulation.

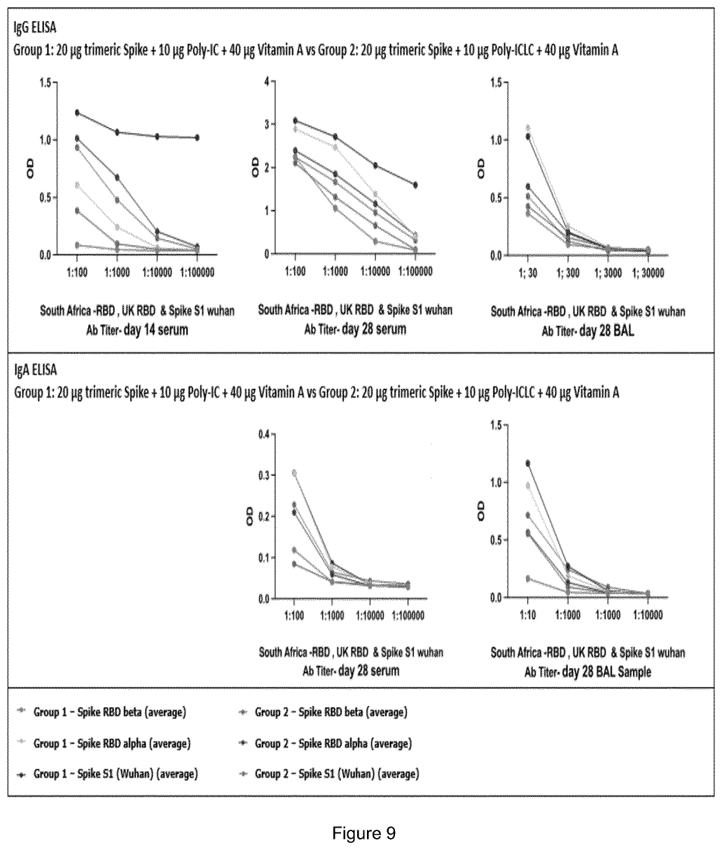

Animals in Group 1 and Group 2 both received 20 μg trimeric SARS-CoV-2 Spike together with 40 μg vitamin A. In Group 1, Poly IC was used as an adjuvant and in Group 2, Poly IC:LC was used as an adjuvant. Two weeks following administration of the first and second dose of test item (i.e., Day 14 and Day 28, respectively), comparable serum IgG and IgA levels were detected in Group 1 and 2. Moreover, comparable IgG and IgA titres were detected in BAL fluid at Day 28. This suggests that the two adjuvants induced a comparable systemic and local antibody response (FIG. 9).

Consequently, Poly IC:LC was considered a suitable adjuvant for development of the Applicant's clinical vaccine candidate.

### Choice of Poly IC:LC Dose

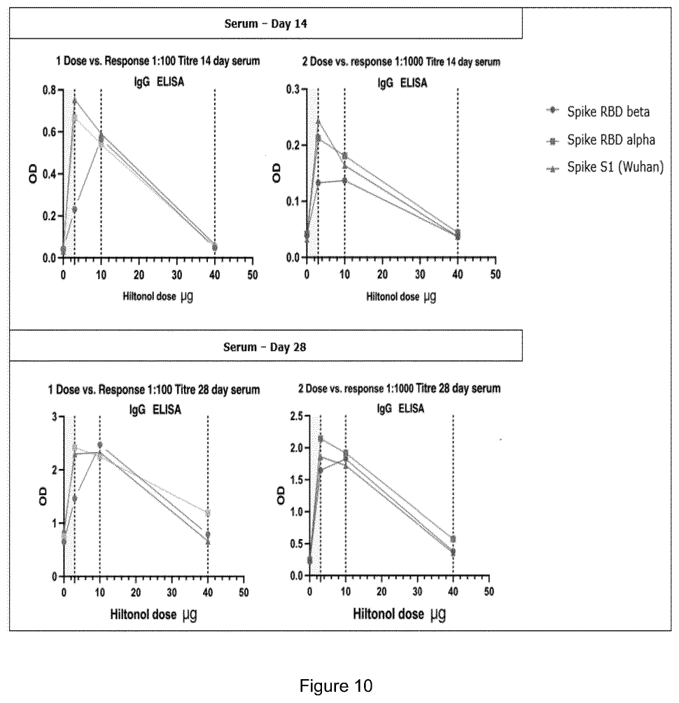

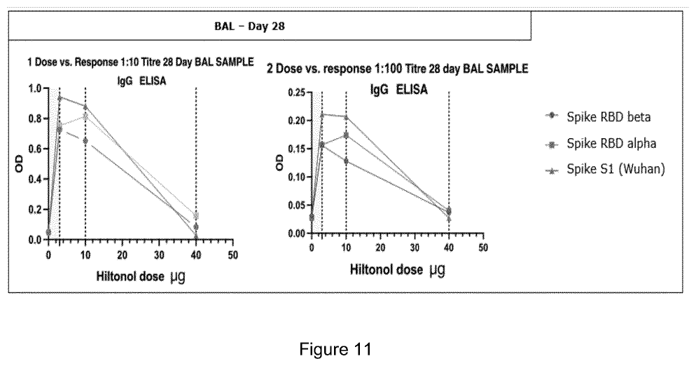

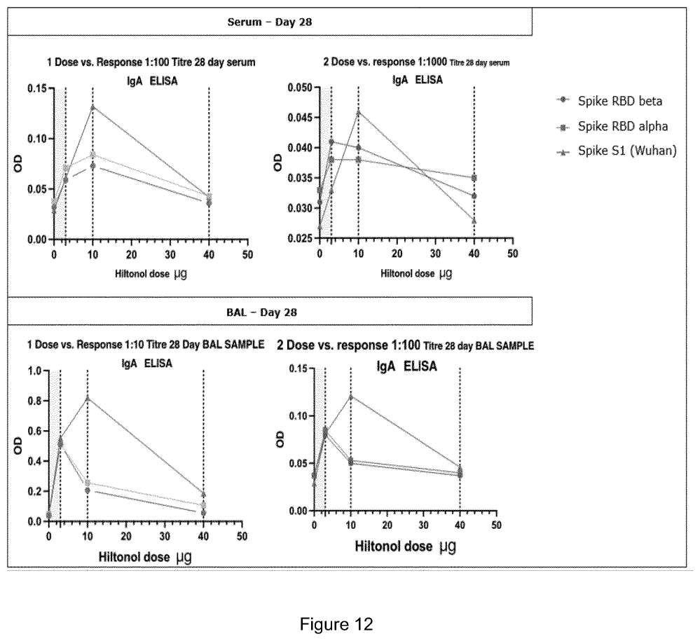

Study AB21-33 also evaluated different doses of the adjuvant Poly IC:LC (3 μg, 10 μg and 40 μg, respectively) as well as test item administration without addition of Poly IC:LC (FIG. 10, FIG. 11 and FIG. 12). Based on the observed dose-response, a Poly IC:LC dose of 10 μg per administration was considered appropriate.

### Vitamin A

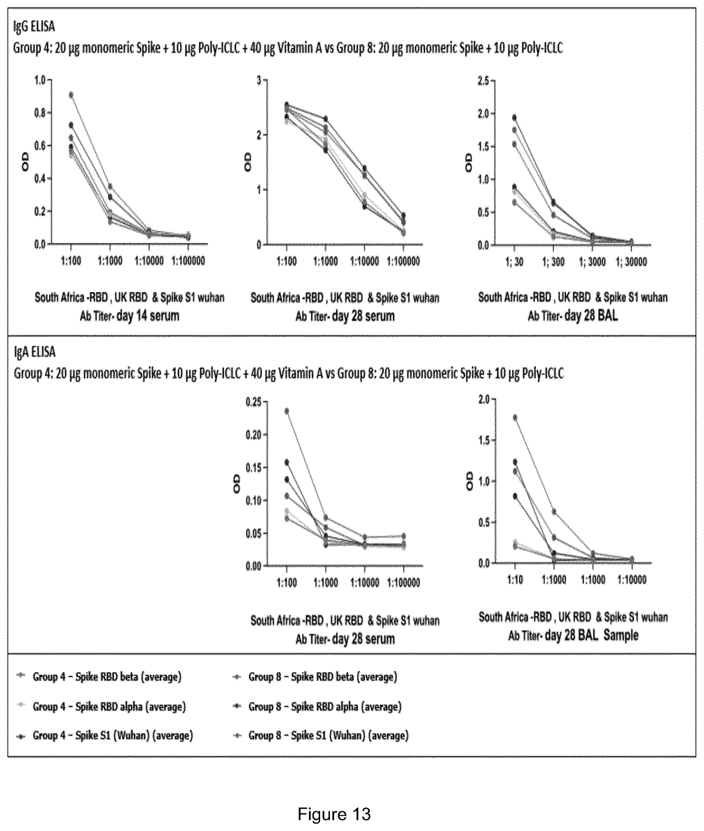

The AB21-33 study also evaluated immunogenicity following vaccination with or without vitamin A. It was hypothesised that addition of vitamin A would enhance IgA production and generally trigger a stronger immune response. Animals in Group 4 were immunised with 20 μg monomeric SARS-CoV-2 Spike together with 10 μg Poly IC:LC and 40 μg Vitamin A; animals in Group 8 also received 20 μg monomeric SARS-CoV-2 Spike together with 10 μg Poly IC:LC but no vitamin A. Serum IgG and IgA titres were compared two weeks following the first and second administration of test item (i.e., Day 14 and Day 28, respectively), and IgG and IgA titres in BAL fluid were compared at Day 28. The results suggest that addition of vitamin A did not further elevate local or systemic IgA titres compared to immunisation with Spike and Poly IC:LC only. Moreover, IgG levels were not affected by addition of vitamin A (FIG. 13). Consequently, vitamin A was omitted in subsequent studies and in the clinical formulation of the candidate vaccine.

### Comparison of Monomeric and Trimeric SARS-CoV-2 Spike

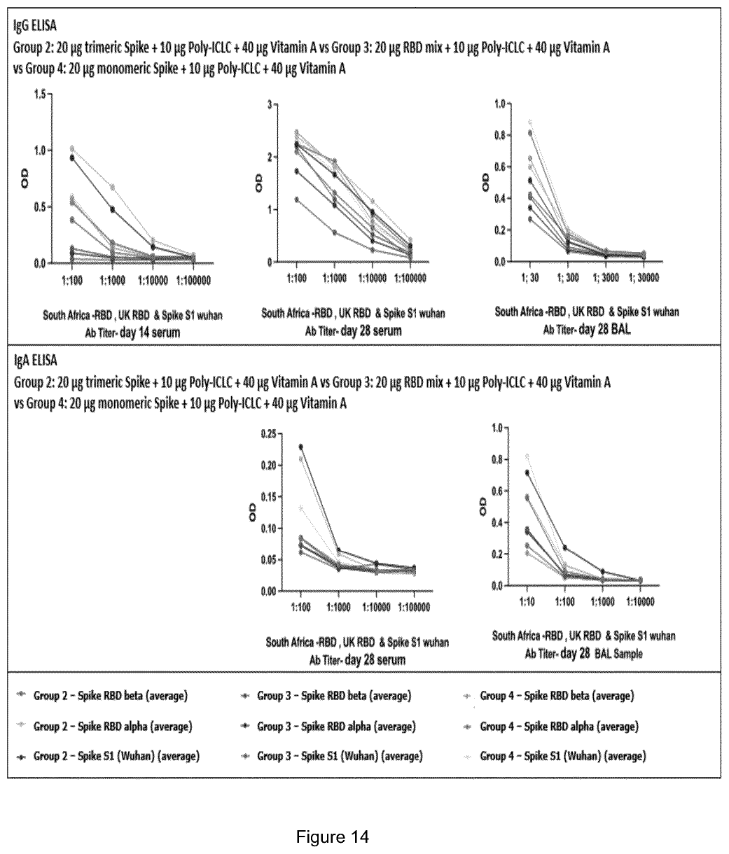

The Applicant's first non-clinical study (AB21-04) used trimeric SARS-CoV-2 Spike as an antigen. However, production of monomeric SARS-CoV-2 Spike would facilitate large-scale production. Therefore, the Applicant evaluated the immune response after stimulation with trimeric (Group 2) and monomeric SARS-CoV-2 Spike S1 (Group 4). Moreover, the immune response following immunisation with a mix of four SARS-CoV-2 RBD2 variants (original strain as well as alpha, beta and gamma variant; Group 3) was analysed. Two weeks following the first and second administration of test item (i.e., Day 14 and Day 28, respectively), serum IgG and IgA were analysed. Furthermore, IgG and IgA titres in BAL fluid were measured at Day 28. The study showed that immunisation with monomeric Spike protein elicited a comparable immune response to immunisation with trimeric Spike protein. Using the different SARS-CoV-2 RBD2 variants as an antigen did not enhance antibody response further (FIG. 14). The Applicant therefore decided to use monomeric SARS-CoV-2 Spike S1 for further development of the candidate vaccine.

### Summary and Conclusion

This study evaluated the immunogenicity of the candidate vaccine in C57BL/6J wildtype mice and provided information that supported the design of following non-clinical studies and the formulation of the clinical candidate vaccine. The impact of using different antigens and adjuvants as well as the impact of adding vitamin A (40 μg) were also evaluated in this study.

The study detected a comparable antibody response when using Poly IC:LC as an adjuvant compared to Poly IC. Moreover, addition of vitamin A did not further elevate local or systemic IgA titres compared to immunisation with Spike and Poly IC:LC only. Similarly, IgG levels were not affected by addition of vitamin A.

Immunisation with monomeric SARS-CoV-2 Spike elicited a comparable immune response as immunisation with trimeric SARS-CoV-2 Spike. Using SARS-CoV-2 RBD mix as an antigen did not further enhance the antibody response.

Based on the results of this study, subsequent studies were performed with monomeric SARS-CoV-2 Spike and the adjuvant Poly IC:LC. Vitamin A was omitted from the following studies as well as from the clinical formulation of the candidate vaccine.

### Example 5—AB21-31 a Second Study to Assess the Efficacy of a COVID-19 Vaccine Against SARS-CoV-2 Infection in hACE2 Transgenic Mice

The aim of this study was to evaluate the efficacy of the candidate vaccine in hACE2 transgenic mice. The study was approved by the regional animal experimental ethics committee in Stockholm (North), Sweden.

Fifty-five female AC70 hACE2 transgenic mice were divided into 5 groups of 11 animals/group. One group served as control and remained untreated. The remaining mice received LD (5 μg) or HD (20 μg) of a 50:50 mix of monomeric SARS-CoV-2 Spike S1 alpha and SARS-CoV-2 Spike S1 beta together with 10 μg of the adjuvant administered via the i.n. or i.t. route on Days 0 and 14 (Table 4). While the previous study, AB21-04, used Poly IC as an adjuvant, study AB21-31 and subsequent studies were conducted with Poly IC:LC, a Poly IC analogue that has been developed for use in clinical settings.

TABLE 3
Treatment schedule in Study AB21-31.

| | | Dose | | Number of |
|---|---|---|---|---|
| | | volume | Route of | animals |
| Group | Treatment | (μl) | administration | (n) |
| 1 | Control (untreated) | — | — | 11 |
| 2 | 5 μg Monomer Spike alpha & beta (LD) + | 26 | i.n. | 11 |
| | 10 μg Poly IC:LC | | | |
| 3 | 20 μg Monomer Spike alpha & beta (HD) + | 26 | i.n. | 11 |
| | 10 μg Poly IC:LC | | | |
| 4 | 5 μg Monomer Spike alpha & beta (LD) + | 26 | i.t. | 11 |
| | 10 μg Poly IC:LC | | | |
| 5 | 20 μg Monomer Spike alpha & beta (HD) + | 26 | i.t. | 11 |
| | 10 μg Poly IC:LC | | | |

HD: High-dose. LD: Low-dose. i.n.: intranasal. i.t.: intratracheal.

On Day 28, animals were challenged with SARS-CoV-2 (1.05×106 TCID50) via i.n. administration. Blood samples for serum isolation were taken pre-vaccination, on Day 14, on Day 28 and at termination. At termination, animals were euthanised and BAL was performed. The spleen was harvested and splenocytes were isolated for T-cell analysis. Lungs (including trachea) and skull (for brain and nasopharyngeal tissues) were collected for histopathological analysis. Animals were weighed and monitored for changes in health status according to the modified Irwin Screen as detailed for study AB21-04 daily until Day 39 (i.e., 11 days post-infection [p.i.]). Four animals in Group 3 died unexpectedly on Day 31, most likely due to not inserting the cage properly back into the rack. These animals were excluded from the p.i. analysis.

### Body Weight and Health Status

Vaccination per se did not overtly affect body weight of the animals. Animals in all groups showed a general increase in body weight following the second vaccination.

Following inoculation with SARS-CoV-2, non-vaccinated animals dropped to 80.88±0.97% of their pre-infection body weights by Day 32. Vaccinated animals, however, remained between 86.16 and 95.07% (mean) of their original body weight Vaccination per se did not overtly affect body weight of the animals. Animals in all groups showed a general increase in body weight following the second vaccination.

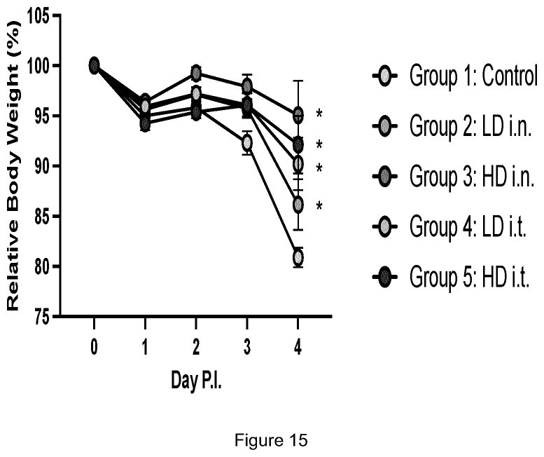

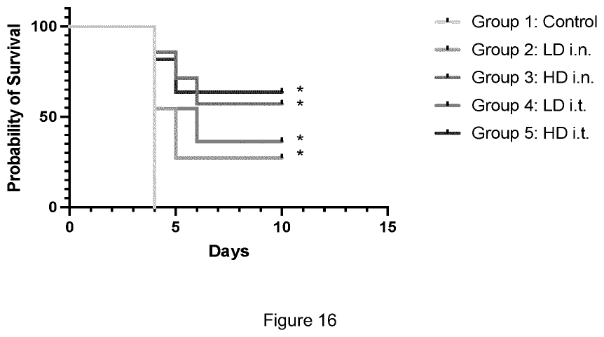

Following inoculation with SARS-CoV-2, non-vaccinated animals dropped to 80.88±0.97% of their pre-infection body weights by Day 32. Vaccinated animals, however, remained between 86.16 and 95.07% (mean) of their original body weight (FIG. 15). On Day 32, relative body weights of the non-immunised controls were significantly lower compared to immunised groups.

Differences in body weight were also evident between immunised groups. A dose-dependent effect was observed, with HD groups showing a slower decline in body weight. By Day 32, 63% of animals in Group 2 (LD, i.n.) had lost >15% of their body weight, compared to only 14% of animals in Group 3 (HD, i.n.). A similar effect was seen for i.t. administration, where 45% of Group 4 (LD, i.t.), but only 27% of Group 5 (HD, i.t.) had lost >15% of their body weight.

Test item administration did not overly affect individual animal health status and did not have any effect on respiratory function. Inoculation with 1.05×106 TCID50 SARS-CoV-2 was associated with a severe decline in health status in non-vaccinated animals. In conjunction with a rapid decrease in body weight, non-vaccinated animals showed clear signs of deteriorating health status from 3 days p.i.

By Day 32 (4 days p.i.), all animals presented with hunched backs. Ten of the animals also had moderate to severe reductions in mobility, and five of the animals presented with rigidity, asymmetry, and trouble coordinating movements. One of the animals also displayed overexcitability, a likely indication of viral invasion into the CNS. One animal lay on its side and appeared to be seizing. All non-vaccinated animals were euthanised pre-term on Day 32 following the severe decline in health status. As compared to the previous study, AB21-04, where animals were inoculated with 1.875×105 TCID50 (passage 3), a more severe impact on the animals' health status was evident.

Symptoms associated with SARS-CoV-2 infection were also observed in a subset of vaccinated animals, specifically between Days 32 and 34 (4-6 days p.i.). In general, health status deteriorated more slowly in vaccinated animals, with an initial phase of overexcitability, followed by clear signs of illness. Fifty-four percent of animals in Group 2 (LD, i.n.) presented with hunched backs, some animals also displayed rigidity, asymmetry, overexcitability and/or reduced mobility. One animal in this group was found dead on Day 33. Forty-two percent of animals in Group 3 (HD, i.n.) displayed particularly severe symptoms, with one animal showing complete loss of coordination and one animal unable to right itself. In Group 4 (LD, i.t.), 72% of animals presented with adverse symptoms. In two of the animals, these were mild and resolved. For the remaining animals in this group (63%), the symptoms were severe enough to warrant pre-term euthanasia. Group 5 (HD, i.t.) had the fewest number of animals (36%) exhibiting SARS-CoV-2-related symptoms such as hunched backs, reduced mobility, rigidity, asymmetry and/or overexcitability leading to pre-term euthanasia.

### Survival Following Viral Challenge

Test item administration significantly improved survival following viral challenge (p=0.0007; Error! Reference source not found.). Median survival for non-vaccinated animals was 4 days, which was significantly shorter compared to survival for animals in Group 2 (LD, i.n.; median survival: 5 days, p=0.005), Group 3 (HD, i.n.; median survival: undefined, p=0.0003), Group 4 (LD, i.t.; median survival: 6 days, p=0.005) and Group 5 (HD, i.t.; median survival: undefined, p=0.0001).

### Antibody Responses (Serum)

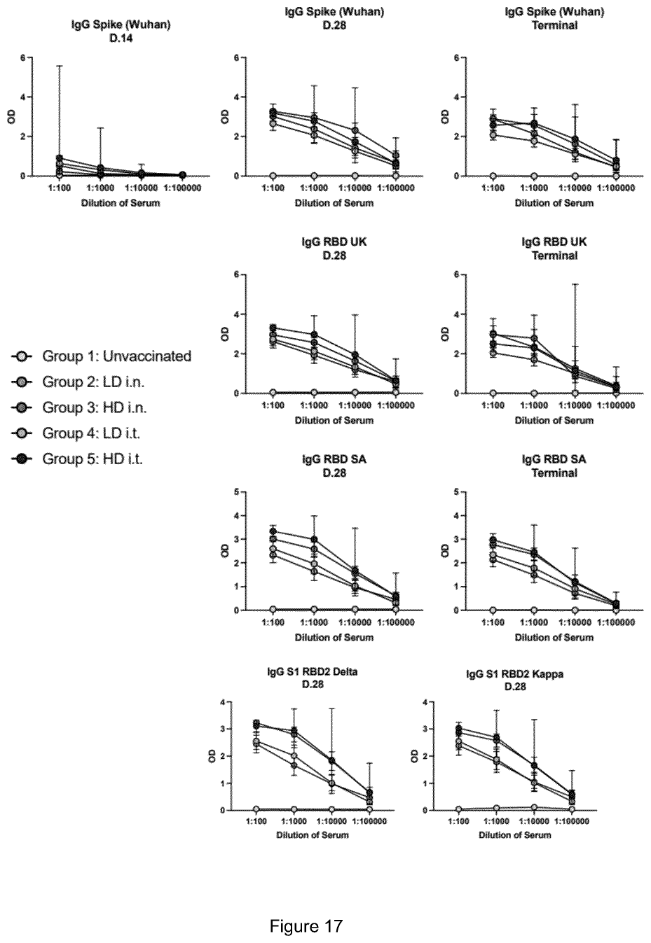

Circulating IgG titres against Spike (from the original strain) were detected in all vaccinated groups by Day 14 and increased following the second vaccination. No further increase was evident upon termination (FIG. 17). For all groups, titres were significantly elevated above non-vaccinated control animals. Increases were dose-dependent, with animals in HD groups showing more consistent and higher antibody titres compared to animals in LD groups. Similar patterns were evident for IgG titres against RBD (alpha) and RBD (beta). IgG antibody titres against S1 RBD2 delta and S1 RBD2 kappa were also detectable in serum on Day 28, with levels significantly elevated above control animals. A dose dependent effect was evident; no differences between administration routes could be seen (FIG. 17).

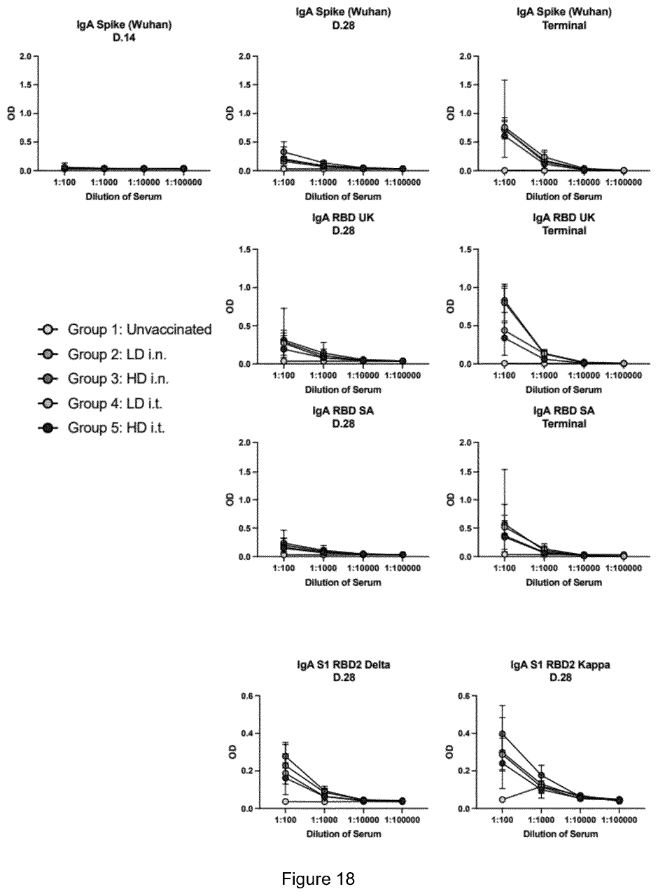

On Day 14, IgA titres against Spike (from the original strain) were mainly evident in Group 5 (HD, i.t.); by Day 28, they were detectable in all vaccinated groups. A further increase was evident at termination (FIG. 18). Anti-Spike IgA titres were significantly elevated compared to control animals for animals in HD groups (Group 3 and Group 5) on Day 28 and in all groups at termination. In comparison, significant increases in IgA titres against RBD (alpha and beta) were only evident in samples from animals that had the vaccine administered i.n. (Groups 2 and 3) on Day 28 but in all groups at termination. The IgA antibody titres against S1 RBD2 delta and S1 RBD2 kappa were detectable in serum on Day 28. For the delta variant, levels were only significantly elevated above control for i.n. groups; for the kappa variant, significant elevations were evident in the HD i.n. group (Group 3) (FIG. 18).

### Antibody Responses (BAL)

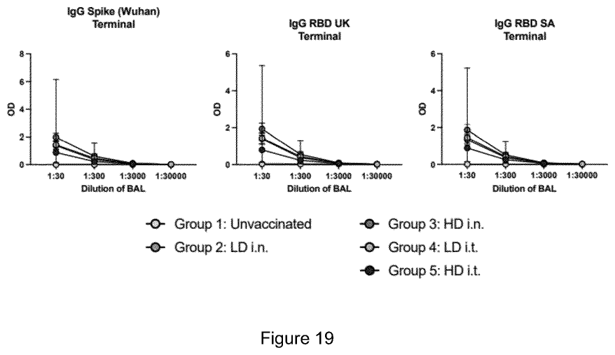

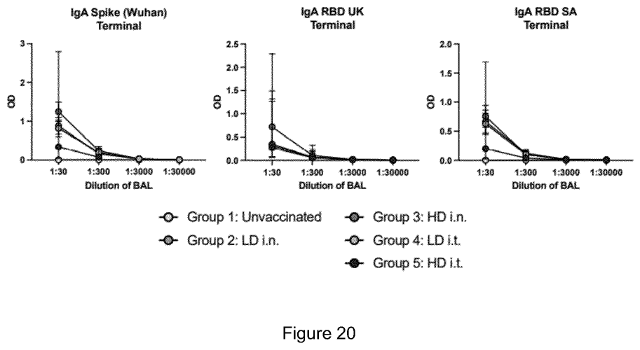

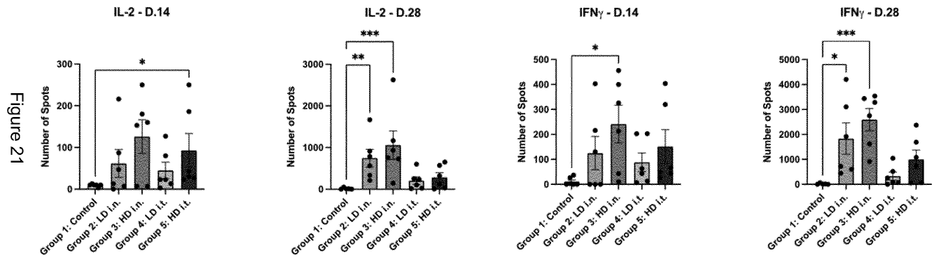

The IgG and IgA titres against Spike (from the original strain), RBD (alpha) and RBD (beta) were detected in all vaccinated groups, at levels significantly higher than control animals. No significant or observable differences were evident between vaccinated groups (FIG. 19 and FIG. 20).

### Neutralising Antibody Titres

Neutralising antibodies against SARS-CoV-2 Spike of the original strain from Wuhan, China, were not detected in BAL of control animals. In comparison, neutralising antibodies (titres ranging from <1.4 to 1:8) were detected in a subset of vaccinated animals: three animals in Group 2, four animals in Group 3, four animals in Group 4 and six animals in Group 5. A dose-dependent response was evident and neutralising antibodies were more frequently detected in animals in the HD groups. A statistically significant correlation was evident between neutralisation titres and survival (r=0.8811, p<0.0001).

Neutralising antibody titres against the beta and delta variants of SARS-CoV-2 were evaluated in pooled serum samples collected on Day 28. Neutralising antibodies against the SARS-CoV-2 beta variant were detected in all immunised groups, whereas neutralising antibodies against the delta variant were only detected in the HD i.n. group and in animals that were immunised via the i.t. route (LD and HD). A dose response was evident, with animals in HD groups having higher titres: Neutralising antibodies against the SARS-CoV-2 beta at titres of 1:128 were detected in the pooled serum of the HD i.t. and HD. i.n. groups, respectively, compared to 1:32 in the LO i.t group and 1:32 to 1:64 in the LD i.n. group. The detected titres in immunised animals were similar or higher compared to the titres measured in the serum of convalescent Covid-19 patients in the same analysis. In contrast, neutralising antibodies were not detected in control animals.

### T-cell Responses

The T-cell assay failed as the cells showed poor viability when the experiment was conducted. No signal was detected in the Fluorospot assay.

### Viral Titres

Viral titres in BAL fluid were assessed by qPCR. As expected, virus was detectable in non-vaccinated control animals. However, in one control animal, no virus was detectable; the reason for this is unclear.

In vaccinated animals, viral replicates were generally low or undetectable. However, in a subset of animals in each group, C(t) values similar to control animals were measured. The number of animals with low or undetectable viral titres were generally higher in HD groups, and in animals vaccinated via the i.t. route. As with neutralisation titres, viral load significantly correlated with survival (r=0.7696, p<0.0001).

### Histopathology

Inflammatory changes were observed in the respiratory tract in all groups. Perivascular and peribronchial inflammatory cell infiltration was evident in the lungs. The severity was slightly higher in vaccinated groups; this may be evidence of a vaccine-driven anti-viral immune response.

### Summary and Conclusion

Intranasal inoculation with 1.05×10⁶ TCID50 SARS-CoV-2 resulted in a rapid decline in body weight, and severe deterioration in health status, resulting in pre-term euthanasia within four days of infection. This was associated with increased viral titres in the lower respiratory tract. Intranasal and i.t. administration of monomeric Spike protein (LD: 5 μg or HD: 20 μg) and Poly IC:LC (10 μg) had no overall effect on health status.

Vaccinated animals showed a progressive and dose-dependent serological response, with systemic and local production of IgG and IgA antibodies against Spike and RBD, as well as moderate local production of neutralising antibodies. This was associated with reduced viral replication in the lungs, and partial prevention of Covid-19 disease progression. Histopathology data will also be evaluated for this study but is not available yet.

In summary, the data from this study suggest that i.n. and i.t. administration of 20 μg monomeric Spike S1 (50:50 mix of alpha and beta variant) together with 10 μg Poly IC:LC on two occasions provides dose-dependent protection against a severe infection following inoculation with 1.05×10⁶ TCID50 SARS-CoV-2.

### Example 6—AB21-73 a Follow-Up Study to Determine Immunogenicity of a Novel Vaccine Against Covid-19 in Wildtype Mice

Study AB21-73 evaluated immunogenicity of the antigen (5 μg or 20 μg monomeric SARS-CoV-2 Spike S1 alpha) and the adjuvant (10 μg Poly IC:LC) of the clinical formulation of the Covid-19 candidate vaccine in C56BL/6J wildtype mice. Primarily, the study focused on evaluating a possible CD4⁺ and CD8⁺ T-cell response (T-cell immunogenicity) elicited by the test item. One or two doses of the test item were administered via the i.n. and i.t. route, respectively. In this study, 60 female C57BL/6J mice were divided into two cohorts (A and B) of 30 animals each. Each cohort was subdivided into five treatment groups with 6 animals/group (Table 5).

TABLE 4
Treatment schedule in AB21-73.

| | | | Dose volume | Route of | Number of |
|---|---|---|---|---|---|
| Cohort | Group | Treatment | (μL) | administration | animals (n) |
| A | 1 | Control (untreated) | — | — | 6 |
| | 2 | 5 μg Monomer Spike alpha + | 25 | i.n. | 6 |
| | | 10 μg Poly IC:LC | | | |
| | 3 | 20 μg Monomer Spike alpha + | 25 | i.n. | 6 |
| | | 10 μg Poly IC:LC | | | |
| | 4 | 5 μg Monomer Spike alpha + | 25 | i.t. | 6 |
| | | 10 μg Poly IC:LC | | | |
| | 5 | 20 μg Monomer Spike alpha + | 25 | i.t. | 6 |
| | | 10 μg Poly IC:LC | | | |
| B | 1 | Control (untreated) | 25 | — | 6 |
| | 2 | 5 μg Monomer Spike alpha + | 25 | i.n. | 6 |
| | | 10 μg Poly IC:LC | | | |
| | 3 | 20 μg Monomer Spike alpha + | 25 | i.n. | 6 |
| | | 10 μg Poly IC:LC | | | |
| | 4 | 5 μg Monomer Spike alpha + | 25 | i.t. | 6 |
| | | 10 μg Poly IC:LC | | | |
| | 5 | 20 μg Monomer Spike alpha + | 25 | i.t. | 5 |
| | | 10 μg Poly IC:LC | | | |

i.n.: intranasal;
i.t.: intratracheal

Animals in Group 1 of both cohorts remained untreated; all other groups were vaccinated on Day 0. Animals in Group 2-5 of Cohort B received a second dose of the candidate vaccine on Day 14. Blood samples for serum isolation were collected on Days 0 and 17 (Cohort A) or Days 0, 14 and 28 (Cohort B). Animals in Cohort A were terminated on Day 17 and animals in Cohort B were terminated on Day 28. At termination, BAL was performed, and spleens were harvested. IgG and IgA titres against Spike RBD2 (alpha) and Spike RBD2 (delta) were measured in serum and BAL fluid. CD4 and CD8 T-cell responses in splenocytes were determined by fluorescence-activated cell sorting (FACS). Body weight and health status were recorded on vaccination days and at termination.

### T-Cell Immunogenicity

Two immunisations (both i.n. and i.t.) led to a robust SARA-CoV-2 specific T-cell response measured with IL-2 and IFNg enzyme-linked immunospot (ELISpot) and intracellular IFNg flow cytometry analysis. T-cell responses measured with ELISpot were already observed following a single immunisation and a stronger response was seen following administration of a second dose. Both CD4⁺- and CD8⁺-T-cell IFNg responses were observed two weeks following administration of the second dose (i.e. on Day 28). The CD8*-T-cell response was more pronounced than the CD4⁺-T-cell response. The strongest response was observed in the HD i.n. group; this was the case after administration of one dose as well as after administration of two doses (FIG. 21).

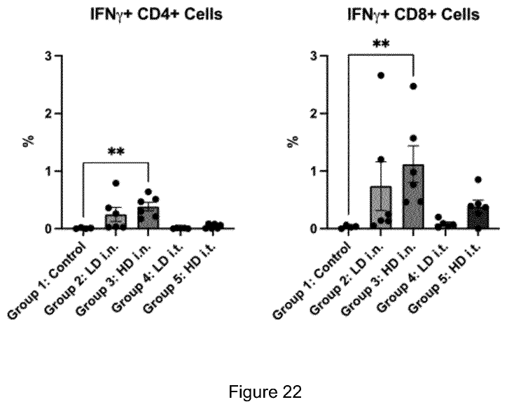

Similarly, intracellular flow cytometry staining revealed significant increases in IFNg⁺-cells following i.n. but not i.t. administration of the test item. Increases in both CD4⁺ and particularly CD8⁺ T-cells were evident (FIG. 22).

### Body Weight and Health Status

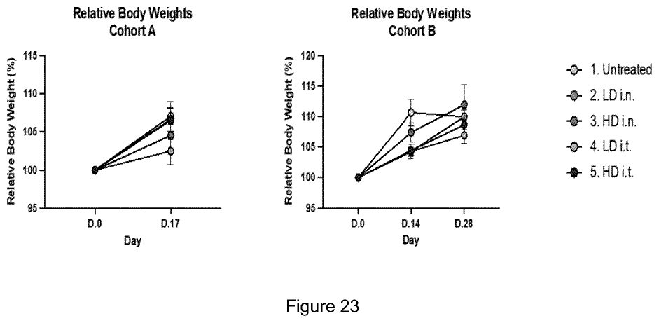

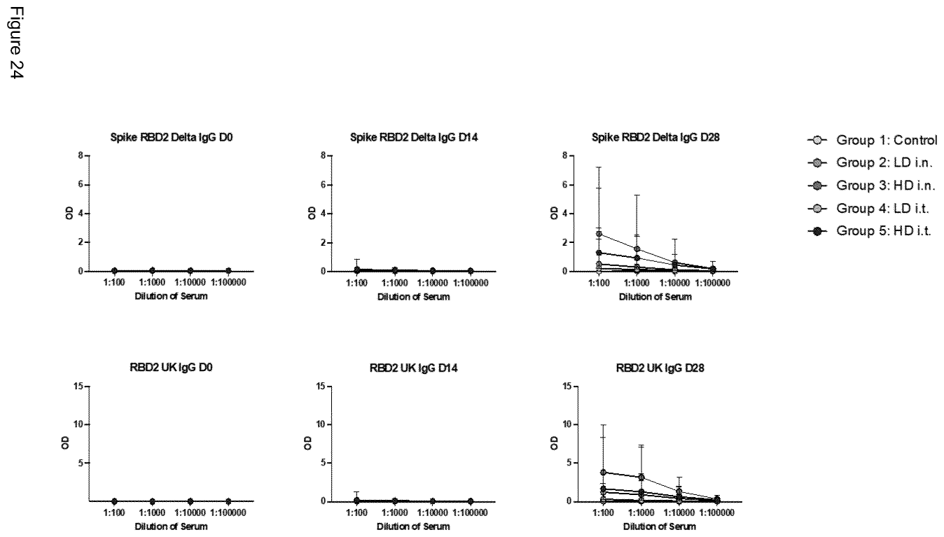

Test item administration was well tolerated and except for a slight transient decrease in body weight in one of the groups receiving i.n. administration, animals were exhibiting normal weight gain (FIG. 23). No overt changes in overall animal health status or respiratory function were observed following administration of the test item.

### Antibody Responses (Serum)

High IgG titres against anti-SARS-CoV-2 RBD2 alpha were detected after two immunisations with the HD formulation. Administration of LD test item only triggered robust responses when administered as two doses via the i.n. route. After the first immunisation, generally only very low IgG responses were observed. However, two animals in the HD i.n. group developed high antibody levels already after the first immunisation. Cross-reactivity of the IgG antibodies with SARS-CoV-2 Spike RBD2 delta was observed (as determined in ELISA measurements) (FIG. 24).

### Antibody Responses (BAL)

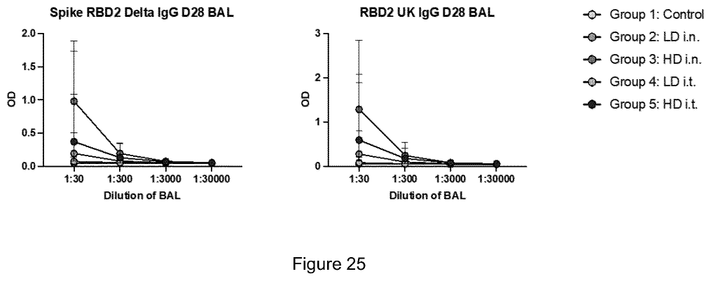

Intranasal administration of HD test item also triggered IgG and IgA secretion in the lung two weeks following administration of the second dose. Cross-reactivity of the immunoglobulins was observed as these targeted both SARS-CoV-2 Spike RBD alpha and SARS-CoV-2 Spike RBD beta. Administration of HD via the i.t. route resulted in a weaker response and was only evident in some of the animals. Following administration of LD test item, only weak antibody responses were observed, and these were only evident in some of the animals (primarily those that were immunised via the i.n. route) (FIG. 25).

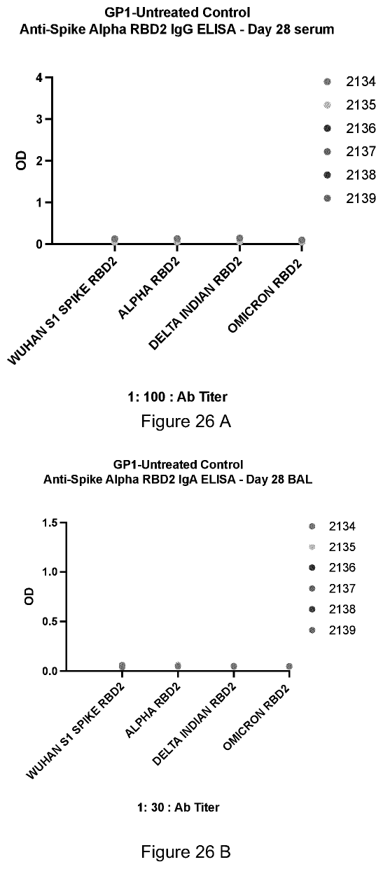

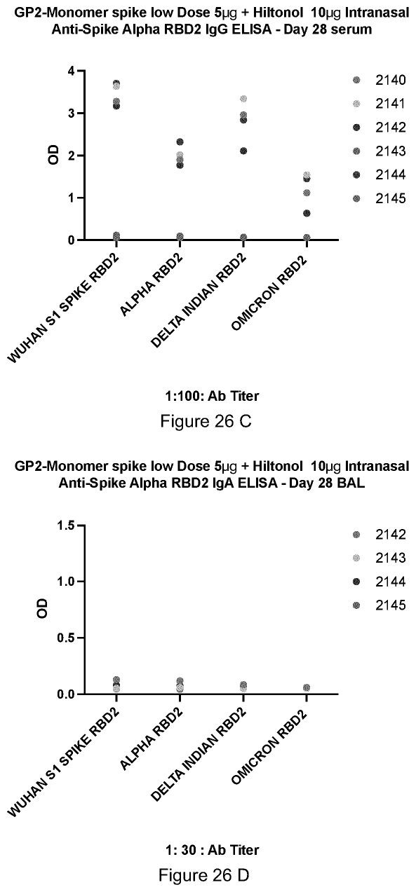

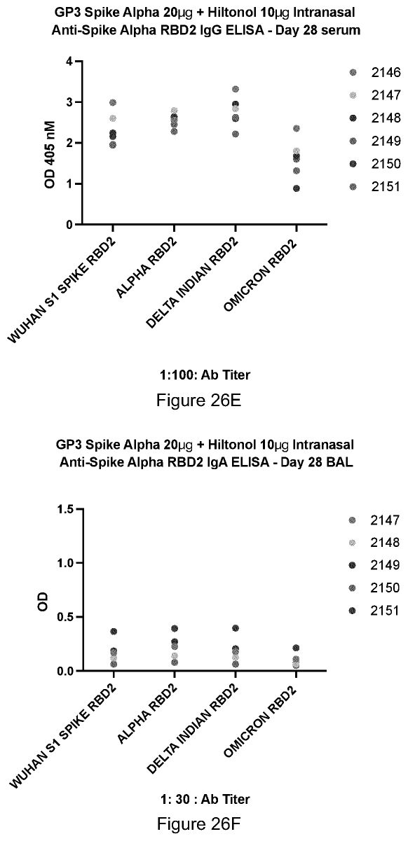

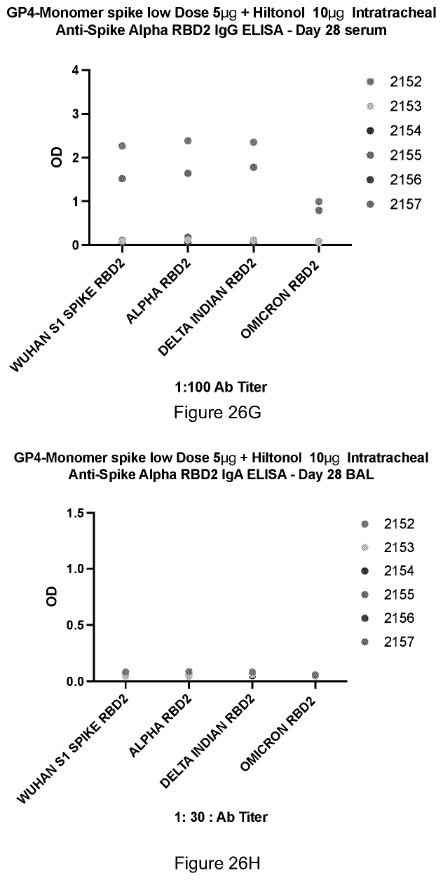

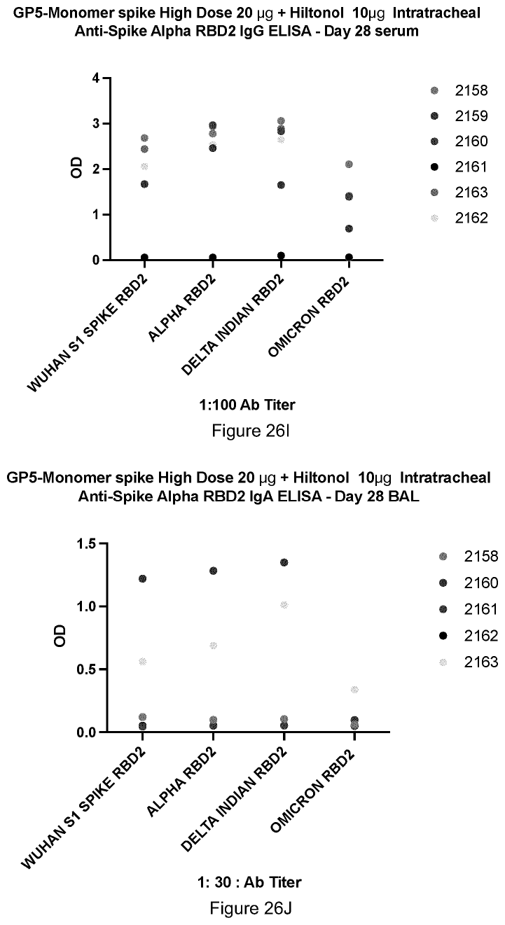

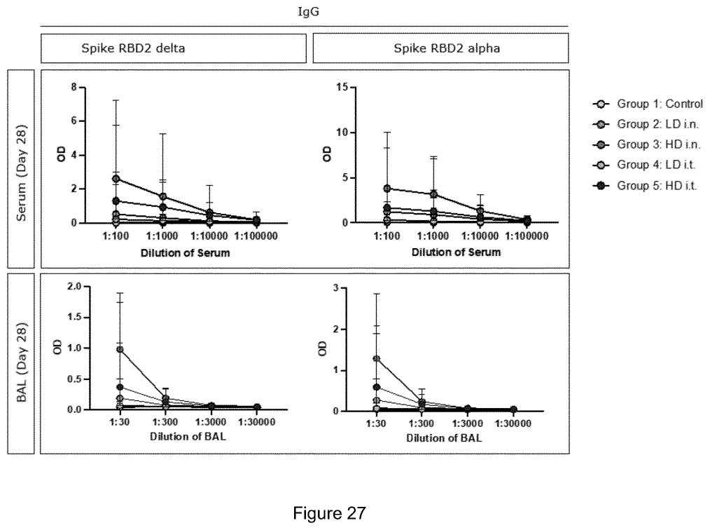

In FIGS. 26A-J are shown ELISA results showing cross reactivity with Spike Wuhan, Alpha, Delta and Omicron.

### CONCLUSION

In conclusion, the data from this study suggests that administration of two doses of 5 μg or 20 μg SARS-CoV-2 Spike S1 alpha (5-20 μg) together with 10 μg Poly IC:LC elicits broad humoral and cell-mediated immune responses against the antigenic Spike and RBD components of SARS-CoV-2, without adversely affecting animal health.

Test item administration induced a T-cell response with both CD4⁺ and CD8⁺ T-cells. The strongest and most robust immune response was triggered following i.n. administration. Moreover, immunisation induced local and systemic production of IgG antibodies.

### Example 7—AB21-74 a Follow-Up Study to Determine the Long-Term Immunogenicity of a Novel Vaccine Against Covid-19 in Wildtype Mice

The Sponsor is currently conducting one additional non-clinical study with the antigen and adjuvant of the candidate vaccine; the study started in September 2021 and is expected to be completed in March 2022.

Study AB21-74 aims to evaluate the long-term immunogenicity of 5 μg or 20 μg monomeric SARS-CoV-2 Spike S1 alpha and 10 μg Poly IC:LC in wildtype mice. Two doses of the test item will be administered via the i.n. and i.t. route, respectively. In this study, 40 female C57BL/6J mice will be divided into four treatment groups of ten animals each (Table 6).

TABLE 5
Treatment schedule in AB21-74.

| | | Dose | Route of | Number of |
|---|---|---|---|---|
| | | volume | adminis- | animals |
| Group | Treatment | (μl) | tration | (n) |
| 1 | 5 μg Monomer Spike + | 25 | i.n. | 10 |
| | 10 μg Poly IC:LC | | | |
| 2 | 20 μg Monomer Spike + | 25 | i.n. | 10 |
| | 10 μg Poly IC:LC | | | |
| 3 | 5 μg Monomer Spike + | 25 | i.t. | 10 |
| | 10 μg Poly IC:LC | | | |
| 4 | 20 μg Monomer Spike + | 25 | i.t. | 10 |
| | 10 μg Poly IC:LC | | | |

i.n.: intranasal. i.t.: intratracheal.

Animals will be vaccinated on Days 0 and 14. Blood samples for serum isolation will be collected pre-vaccination, on Days 14, 28, 42 and 98 as well as at termination (Day 182, i.e., 24 weeks after the second vaccination). At termination, BAL will be performed for determination of anti-Spike and anti-RBD IgG and IgA titres; the spleen will be harvested for T-cell (CD4 and CD8) analysis. Body weights and health status will be recorded every 14 days.

### Safety Pharmacology

No non-clinical stand-alone studies on safety pharmacology have been conducted. However, safety pharmacology observations were performed as part of the non-clinical in vivo studies conducted to date. The findings from these studies do not suggest any adverse effects of the candidate vaccine on vital organs or functions.

### Pharmacology Discussion and Conclusion

Immunogenicity with different antigens (monomeric or trimeric SARS-CoV-2 Spike S1 of the original strain, SARS-CoV-2 Spike S1 alpha or beta, and SARS-CoV-2 Spike-RBD of the original strain as well as alpha, beta and gamma) has been shown in the Sponsor's non-clinical studies. In these studies, immunogenicity was assessed in ELISA measurements to get indications on the production of local (BAL fluid) and systemic (serum) IgG and IgA levels, as well as in viral neutralization assays (VNA) to assess the ability of the antibodies to neutralize different SARS-CoV-2 strains. Moreover, the cellular immunity was assessed following immunization with SARS-CoV-2 Spike S1 alpha (i.e., the antigen used in the clinical formulation of the candidate vaccine). The results from these assessments are summarized below.

### ELISA

Presence of IgG and IgA following immunisation were determined using the ELISA method. Plates were coated with the respective antigen (Spike S1 of the original strain, Spike RBD alpha, Spike RBD beta, Spike RBD2 delta or Spike RBD2 kappa) and different dilutions of serum or BAL samples were evaluated.
1. Following immunisation with trimeric SARS-CoV-2 Spike S1 (original strain) via the i.n. and i.t. route in study Error! Reference source not found., anti-Spike S1 (original strain), anti-RBD alpha and anti-RBD beta IgG and IgA antibodies were detected in the serum and BAL fluid of vaccinated animals suggesting cross-reactivity. Local and systemic IgG production was dose-dependent, with animals that had received a higher dose of Spike showing a stronger immunological response. For IgA, local but not systemic response was dose dependent. Immunisation with monomeric or trimeric Spike S1 (original strain) or with the RBD mix in study Error! Reference source not found. showed similar results.
2. Cross-reactivity of IgA and IgG was also observed in studyAB21-31. In this study, animals were immunised with monomeric SARS-CoV-2 Spike S1 (50:50 alpha and beta) via i.n. and i.t. administration. This led to dose-dependent production of circulating anti-Spike S1 (original strain), anti-RBD alpha and anti-RBD beta IgG in all vaccinated groups. Moreover, anti-Spike S1 (original strain), anti-RBD alpha and anti-RBD beta IgA and IgG were detected in BAL fluid of all vaccinated groups. Cross-reactivity is further reemphasized by the presence of IgG and IgA antibodies against S1 RBD2 delta and S1 RBD2 kappa in the serum of immunized mice.
3. Immunisation with SARS-CoV-2 Spike S1 alpha (i.e., the antigen used in the clinical candidate vaccine) in study AB-21-73 induced production of anti-SARS-CoV-2 Spike RBD2 alpha and delta IgG in serum and BAL fluid. Both i.t. and i.n. administration induced a dose-dependent increase in circulating IgG titres against Spike RBD2 alpha and delta following administration of two doses. Administration of a single dose only led to significantly increases in IgG titres in the HD group which received the test item via the i.t. route. Moreover, two doses of the test item (i.n. and i.t.) were also associated with locally increased anti-Spike RBD2 (alpha) and anti-Spike RBD2 (delta) IgG titres in BAL fluid. Increases in IgG titres were significant in HD groups and were most pronounced following i.n. administration (Error! Reference source not found.).

### VNA

In the VNA assays, VeroE6 cells were seeded on 96-well plates at a seeding density of 2×104 cells per well one day prior to infection. BAL or serum was heat inactivated for 30 minutes at 56° C. before dilution in Dulbecco's Modified Eagle Medium supplemented with 2% foetal calf serum, 100 units/mL of penicillin and 100 μg/mL streptomycin. The diluted BAL or serum was then distributed in triplicates into 96-well round bottom plates and mixed with 500 plaque-forming units (PFU) of SARS-CoV-2 per well and incubated at 37° C./5% CO2 for 1 hour. Media from the VeroE6 cells was aspirated and replaced with virus:BAL/serum mix. Virus only, BAL/serum only, and convalescent patient serum controls were included. After 3 days of incubation at 37° C./5% CO2, the presence of cytopathic effect (CPE) was checked. Neutralising activity was assigned to wells which showed a complete absence of CPE. The neutralising titre was then calculated as the dilution factor where >50% of wells showed neutralising activity.
1. Viral neutralization was evaluated in BAL fluid in AB21-04 following immunization with trimeric SARS-CoV-2 Spike S1 (original strain) via different routes of administration (s.c., i.n. and i.t.). Following s.c. administration, only three animals showed presence of neutralizing antibodies; titres were low (1:4) and partial neutralization was evident in two animals. Intranasal administration was associated with higher neutralizing antibody titres. The response was dose-dependent, with three animals in the LD group presenting with low tires (1:4 to 1:16) and six animals in the HD group presenting with medium to high titres (1:4 to 1:32). Intratracheal administration was also associated with detectable neutralizing antibodies. Two animals in the LD group showed low titres (1:4) and five animals in the HD group showed low to moderate titres (1:4 to 1:8)).
2. Moreover, study AB-21-31 evaluated neutralizing antibodies against SARS-CoV-2 Spike of the original strain from Wuhan, China, following immunization with monomeric SARS-CoV-2 Spike S1 (50:50 alpha and beta) via i.n. and i.t. administration. Neutralizing antibodies were not detectable in BAL of control animals but were present in a subset of vaccinated animals at titres ranging from <1.4 to 1:8. A dose-dependent response was evident and neutralizing antibodies were more frequently detected in animals in the HD groups. Neutralizing antibody titres correlated significantly with survival (r=0.8811, p<0.0001). The study also evaluated neutralizing antibody titres against SARS-CoV-2 beta and delta in pooled serum samples collected on Day 28. Neutralizing antibodies against the SARS-CoV-2 beta variant were detected in all immunised groups; neutralizing antibodies against the delta variant were detected in the HD i.n. group and in animals that were immunised via the i.t. route (LD and HD) but not in the LD i.n. group. A dose response was evident, with animals in HD groups having higher titres: Neutralizing antibodies against the SARS-CoV-2 beta at titres of 1:128 were detected in the pooled serum of the HD i.t. and HD. i.n. groups, respectively, compared to 1:32 in the LD i.t group and 1:32 to 1:64 in the LD i.n. group. Similarly, neutralizing antibody titres against SARS-CoV-2 delta were higher in pooled serum samples of the HD i.t. (1:64) and HD i.n. (1:32 to 1:64) groups, respectively, compared to the LD i.t. (1:16 to 1:32) group. The detected titres in immunised animals were similar or higher compared to the titres measured in the serum of convalescent Covid-19 patients in the same analysis. In contrast, neutralizing antibodies were not detected in control animals. This data provides evidence that immunization with SARS-CoV-2 Spike S1 (50:50 alpha and beta) induces cross-reactive neutralizing antibodies in both BAL and serum.

### T-Cell Immunity

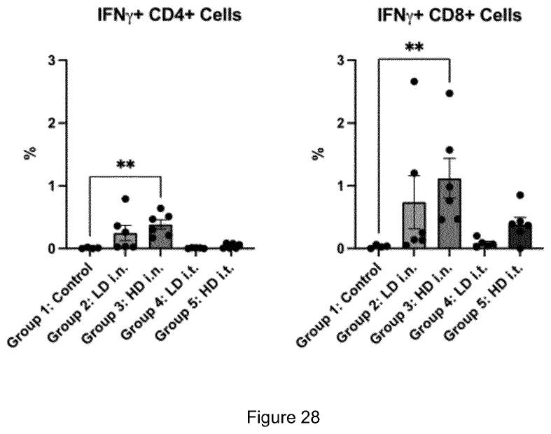

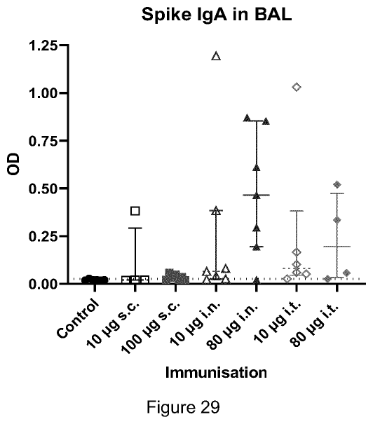

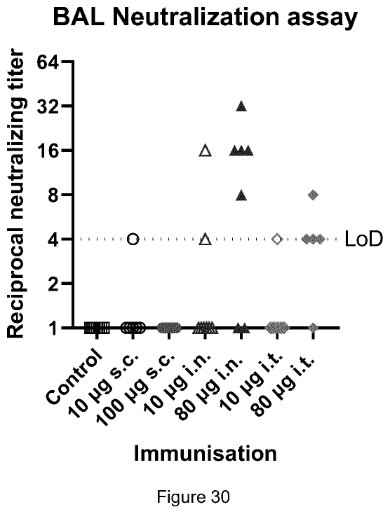

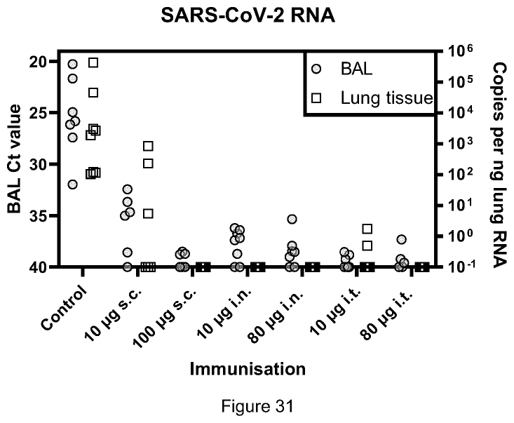

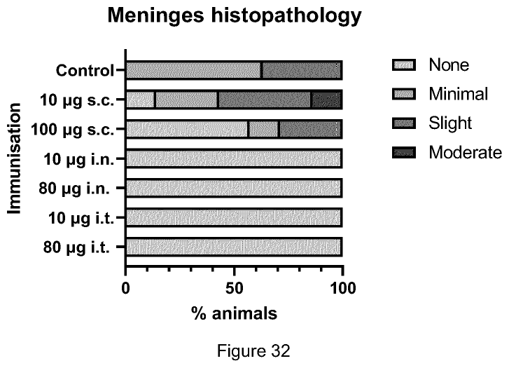

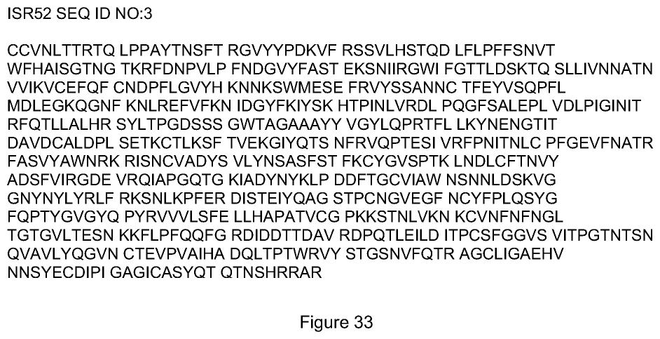

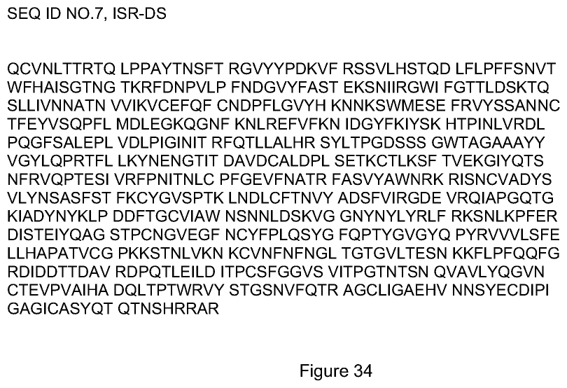

Study AB21-73 evaluated T-cell immunogenicity of 5 μg (LD) or 20 μg (HD) of monomeric SARS-CoV-2 Spike S1 alpha (5 μg and 20 μg) together with 10 μg Poly IC:LC in C56BL/6J wildtype mice. The study showed that both i.n. and i.t. administration of antigen and adjuvant were associated with an increase in SARS-CoV-2-specific CD4⁺ and CD8⁺ T-cells. A significant increase in IFNγ-secreting splenocytes was seen following i.n. but not i.t. administration of the test item. This increase was dose-dependent, and administration of HD resulted in a more pronounced response (FIG. 3). Animals in the HD i.n. group exhibited a significant increase in IFNγ⁺ T-cells already following a single dose of the test item. Moreover, i.n. administration resulted in a significant and dose-dependent increase in IL-2-secreting splenocytes following two administrations of the test item. Intratracheal administration of HD test item resulted in a significant increase in IL-2 secreting splenocytes after a single administration. A further increase was evident following administration of a second dose; however, this increase was not statistically significant (FIG. 28).

In summary, this study suggests that i.n. and i.t. administration of two doses of 5 μg or 20 μg SARS-CoV-2 Spike S1 alpha (5 μg-20 μg) together with 10 μg Poly IC:LC elicits—in addition to a humoral immune response—also a cell-mediated immune response against the antigenic Spike and RBD components of SARS-CoV-2 with a predominance of SARS-CoV-2 reactive CD8⁺ T-cells. This was particularly evident following i.n. administration of 20 μg Spike S1 alpha together with the adjuvant Poly IC:LC.

In addition to the immunogenicity data presented above, long-term data for ISR52 with the alpha variant of the Spike protein as the active substance will be generated in the ongoing long-term immunogenicity study AB21-74.

### Summary and Conclusion

Based on available immunogenicity data together with the cross-reactivity of IgG and IgA antibodies observed in the non-clinical studies, it is expected that immunisation with SARS-CoV-2 Spike S1 alpha as the only antigen has comparable efficacy as immunisation with other Spike variants. The full-length Spike S1 protein used as antigen in the ISR52 vaccine enables broad immune responses towards different Spike variant. Covid-19 epidemiology is continuously changing with the emergence of new SARS-CoV-2 variants. These variants may carry mutations in antigenic regions which constitutes a challenge for vaccine developers. The SARS-CoV-2 alpha variant carries a mutation in the RBD of the Spike protein which increases binding affinity to the ACE2 receptor which may at least partially explain the higher infectivity of this strain compared to the original Wuhan strain (Zahradnik et al., 2021). The Spike alpha protein shares this N501Y mutation in the RBD with the beta, gamma and omicron (B.1.1.529) variants as well as the D614G mutation with the beta, gamma, delta and omicron (B.1.1.529) variants.

### Example 8—Clinical Study

A multi-centre, double-blind, randomised, placebo-controlled, dose-finding FIH study (phase 1/2; C-Vac-052-005) is planned to evaluate safety, tolerability and immunogenicity of ISR52 vaccine with the adjuvant Poly IC:LC. The study will be conducted in Bangladesh; a total of 90 healthy subjects aged 18-59 years will be included and randomised (1:1:1) into one of three dose groups (LD: 30 μg Spike+15 μg Poly IC:LC, ID: 60 μg Spike+30 μg Poly IC:LC, HD: 120 μg Spike+60 μg Poly IC:LC). The subjects in each dose group will be allocated in a 2:1 ratio to the clinical formulation of the candidate vaccine (ISR52) and placebo.

Alternatively, the following will be used:

### Cohort 1

10 μg Spike and 10 μg Poly IC:LC
10 μg Spike and 50 μg Poly IC:LC
50 μg Spike and 10 μg Poly IC:LC
50 μg Spike and 50 μg Poly IC:LC
Control

### Cohort 2

120 μg Spike and 10/50 μg Poly IC:LC

The starting dose in phase 1A of the FIH study will contain 30 μg SARS-CoV-2 Spike S1-alpha protein and two additional dose levels will be evaluated in phase 1B of the study:
Low Dose (LD): 30 μg SARS-CoV-2 Spike S1 alpha and 15 μg Poly IC:LC
Intermediate Dose (ID): 60 μg SARS-CoV-2 Spike S1 alpha and 30 μg Poly IC:LC
High Dose (HD): 120 μg SARS-CoV-2 Spike S1 alpha and 60 μg Poly IC:LC

Two doses of ISR52 or placebo will be administered via the i.n. route on Day 0 and Day 28, respectively. Initially, only subjects allocated to the LD group will receive study treatment (phase 1A). A Data Safety Monitoring Board (DSMB) will review the safety data collected from the first six subjects through Day 7. If no safety concerns are raised by the DSMB, enrolment will continue to 30 subjects. Then, the DSMB will review the safety data to Day 7 of the remaining 24 subjects in this group. If there are no safety concerns, the ID group will enroll the first six subjects and the DSMB will review the safety data to day 7. If no concerns arise, the remaining subjects in this group will be dosed and the DSMB will meet at 30 subjects to review the data on the 24 additional subjects. If again no safety concerns are detected, the first six subjects in the HD group will be dosed. The DSMB will meet on Day 7 after dosing of these six subjects. If there are no safety concerns, the remaining subjects will be dosed and the DSMB will meet at 30 subjects, i.e., a total of 90 subjects in the study, and conclude the safety assessment.

The primary endpoints of this study are to assess frequency of adverse events (AEs) and serious AEs (SAEs) after the first (Day 1 to Day 27) and second dose (Day 28 to Day 56) of ISR52 vaccine administration. The study will also evaluate immunogenicity of ISR52 until 4 weeks following administration of the second dose (Day 56).

1. A carrier composition in particulate form for use as carrier for antigenic material derived from respiratory viruses or respiratory bacteria, wherein the carrier composition comprises one or more pharmaceutically acceptable excipients and an adjuvant, wherein the one or more pharmaceutically acceptable excipients comprises i) a disaccharide selected from trehalose, sucrose, and lactose, ii) a polysaccharide selected from cyclodextrins, and wherein the adjuvant is a TLR agonist.

2. A carrier composition for use according to claim 1 as carrier for antigenic material derived from viruses.

3. A carrier composition for use according to claim 1 or 2, wherein the disaccharide is trehalose, the polysaccharide is hydroxypropyl-beta-cyclodextrin, and the TLR agonist is a TLR3 agonist.

4. A carrier composition for use according to any one of the preceding claims, wherein the concentration of the disaccharide is in a range of from 10% to 60% w/w such as from 30% to 55% w/w or from 40% to 50% w/w.

5. A carrier composition for use according to any one of the preceding claims, wherein the concentration of the polysaccharide is in a range of from 10% to 60% w/w such as from 30% to 55% w/w or from 40% to 50% w/w.

6. A carrier composition for use according to any one of the preceding claims, wherein the concentration of the adjuvant is from 0.1% to 5% w/w.

7. A vaccine composition for use in preventing infection of pathogenic respiratory viruses or pathogenic respiratory bacteria by intra-nasal or pulmonary administration of the composition to a subject, wherein the composition comprises an antigenic material from a pathogenic respiratory virus or pathogenic bacteria, and wherein the composition comprises one or more saccharide and a TLR agonist.

8. A composition for use according to claim 7 in particulate form having a mean particle size in a range of from 2 to 50 μm.

9. A composition for use according to claim 7 or 8, wherein the one or more saccharide is selected from trehalose and a cyclodextrin, or mixtures thereof.

10. A composition for use according to any one of claims 7-9 comprising hydroxypropyl-beta-cyclodextrin.

11. A composition for use according to any one of claims 7-10, wherein the TLR agonist is a TLR3 agonist selected from Poly IC and Poly IC:LC.

12. A composition for use according to any one of claims 7-11, wherein the composition comprises a carrier composition together with the antigenic material derived from a pathogenic respiratory virus or pathogenic respiratory bacteria.

13. A vaccine composition comprising an antigenic material derived from a pathogenic respiratory virus or pathogenic respiratory bacterium and one or more pharmaceutically acceptable excipient, wherein the composition is in particulate form having a mean particle size in a range of from 2 to 50 μm, and wherein the composition comprises one or more saccharides and a TLR agonist.

14. A composition according to claim 13, wherein the one or more saccharide is selected from trehalose and a cyclodextrin, or mixtures thereof.

15. A composition according to claim 13 or 14 comprising hydroxypropyl-beta-cyclodextrin.

16. A composition for use according to any one of claims 13-15, wherein the TLR3 agonist is selected from Poly IC and Poly IC:LC.

17. A vaccine composition according to any one of claims 13-16 comprising one or more pharmaceutically acceptable excipients to ensure flowability, to ensure protein structure, to ensure protein stability, to avoid intra-particle cohesivity, and/or to avoid agglomeration.

18. A vaccine composition according to any one of claims 13-17, wherein the respiratory virus is selected from influenza virus, respiratory syncytial virus, parainfluenza virus, metapneumovirus, rhinovirus, coronavirus, adenovirus, and bocavirus.

19. A vaccine composition according to any one of claims 13-18, wherein the respiratory virus is a coronavirus.

20. A vaccine composition according to any one of claims 13-17, wherein the respiratory bacterium is selected from Streptococcus pneumoniae, Mycoplasma pneumonia, Haemophilus influenza, Chlamydophilia pneumoniae, Chlamydia Psittaci, Moraxella catarrhalis, Mycobacterium tuberculosis, Mycobacterium avis, and Mycobacterium marinum.

21. A vaccine composition according to any one of claims 13-20 designed for nasal administration.

22. A vaccine composition according to any one of claims 13-21, wherein the mean particle size is in a range of from 20 to 50 μm such as from 30 to 40 μm and with a size distribution revealing less than 10% of the particles have a particle size of 10 μm or less.

23. A vaccine composition according to any one of claims 13-20 designed for inhalation.

24. A vaccine composition according to any one of claims 13-20, 23, wherein the mean particle size is 10 μm or less such as at the most 8 μm, at the most 6 μm, at the most 5 μm or in a range of from 1 μm to 5 μm such as in a range of from 3 μm to 5 μm.

25. A vaccine composition according to any one of claims 13-24 having a suitable flowability when measured according to the method described in 2.9.16 of Ph.Eur. 10.0 using a funnel without stem and nozzle 1 with a diameter of 10±0.01 mm.

26. A vaccine composition according to any one of claims 13-25, wherein the one or more pharmaceutically acceptable excipient is selected from cellulose, cellulose derivatives, methylcellulose, hydroxypropyl cellulose, hydroxypropyl methyl cellulose, microcrystalline cellulose, saccharides including monosaccharides, disaccharides, oligosaccharides, polysaccharides, amino acids including peptides, and mixtures thereof.

27. A vaccine composition according to any one of claims 13-26, wherein the one or more pharmaceutically acceptable excipient is selected from disaccharides, oligosaccharides, amino acids, peptides and polypeptides.

28. A vaccine composition according to claim 27, wherein the disaccharides are selected from trehalose, sucrose, lactose.

29. A vaccine composition according to claim 28, wherein the disaccharide is trehalose.

30. A vaccine composition according to claim 27, wherein the oligosaccharide is a cyclodextrin.

31. A vaccine composition according to claim 30, wherein the cyclodextrin is a beta-cyclodextrin such as hydroxypropyl-beta-cyclodextrin.

32. A vaccine composition according to claim 27, wherein the amino acid is selected from leucine or lysine and/or the peptide is selected from tri-leucine or tri-lysine and/or the polypeptide is selected from polyleucine or polylysine.

33. A vaccine composition according to any one of claims 13-32 further comprising an adjuvant.

34. A vaccine composition according to any one of claims 13-33 further comprising a TLR agonist.

35. A vaccine composition according to claim 35, wherein the TLR agonist is a TLR2 agonist and/or TLR3 agonist.

36. A vaccine composition according to claim 34 or 35, wherein the TLR agonist is a TLR3 agonist.

37. A vaccine composition according to any one of claims 33-36, wherein the TLR agonist is a TLR3 agonist selected from Poly IC and Poly IC:LC.

38. A composition according to any one of claims 13-37, wherein the composition comprises a carrier composition according to any one of claims 1-6 together with the antigenic material derived from a pathogenic respiratory virus or pathogenic respiratory bacteria.

39. A vaccine composition according to any one of claims 13-38 either further comprising vitamin A and/or vitamin D, or in combination with separate compositions of vitamin A and/or D.
